(19) United States
(12) Patent Application Publication
(10) Pub. No.: US 2023/0348477 A1
(43) Pub. Date: Nov. 2, 2023

(54) ORGANIC COMPOUND, LIGHT-EMITTING DEVICE, LIGHT-EMITTING APPARATUS, AND ELECTRONIC DEVICE

(71) Applicant: SEMICONDUCTOR ENERGY LABORATORY CO., LTD., Atsugi-shI (JP)

(72) Inventors: Masatoshi TAKABATAKE, Atsugi (JP); Yui YOSHIYASU, Atsugi (JP); Sachiko KAWAKAMI, Atsugi (JP); Takeyoshi WATABE, Atsugi (JP); Nobuharu OHSAWA, Zama (JP); Yuko KUBOTA, Atsugi (JP)

(21) Appl. No.: 18/305,733

(22) Filed: Apr. 24, 2023

(30) Foreign Application Priority Data
Apr. 29, 2022 (JP) 2022-075591
Dec. 7, 2022 (JP) 2022-195343

(51) Int. Cl. C07D 487/04; H10K 85/60; H10K 50/17

(52) U.S. Cl. CPC C07D 487/04; H10K 50/171; H10K 85/626; H10K 85/6572

(57) ABSTRACT

An electron-injection organic compound with low solubility in water is provided. An organic compound represented by General Formula (G1) is provided. In the organic compound, Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring, each of R¹ and R² independently represents hydrogen (including deuterium), an alkyl group having 1 to 6 carbon atoms, a substituted or unsubstituted amino group, a substituted or unsubstituted aryl group having 6 to 13 carbon atoms forming a ring, or a substituted or unsubstituted heteroaryl group having 2 to 13 carbon atoms forming a ring, n represents an integer greater than or equal to 1 and less than or equal to 6, and L is the group represented by General Formula (L-1). In General Formula (L-1), each of R³ and R⁴ independently represents hydrogen (including deuterium) or an alkyl group having 1 to 6 carbon atoms, and k is an integer greater than or equal to 1 and less than or equal to 5.

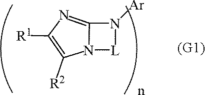

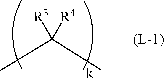

### BACKGROUND OF THE INVENTION

### 1. Field of the Invention

One embodiment of the present invention relates to an organic compound, a light-emitting device, a light-emitting apparatus, a light-emitting and light-receiving apparatus, a display apparatus, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### 2. Description of the Related Art

Recent display apparatuses have been expected to be applied to a variety of uses. Usage examples of large-sized display apparatuses include a television device for home use (also referred to as TV or television receiver), digital signage, and a public information display (PID). In addition, a smartphone and a tablet terminal each including a touch panel, and the like, are being developed as portable information terminals.

Higher-resolution display apparatuses have been required. For example, devices for virtual reality (VR), augmented reality (AR), substitutional reality (SR), or mixed reality (MR) are given as devices requiring high-resolution display apparatuses and have been actively developed in recent years.

Light-emitting apparatuses including light-emitting devices (also referred to as light-emitting elements) have been developed as display apparatuses, for example. Light-emitting devices utilizing electroluminescence (hereinafter referred to as EL; such devices are also referred to as EL devices or EL elements) have features such as ease of reduction in thickness and weight, high-speed response to input signals, and driving with a constant DC voltage power source, and have been used in display apparatuses.

Patent Document 1 discloses a display apparatus using an organic EL device (also referred to as organic EL element) for VR. Patent Document 2 discloses a light-emitting device with a low driving voltage and high reliability in which an electron-injection layer uses a mixed film of a transition metal and an organic compound including an unshared electron pair.

### References

### Patent Documents

[Patent Document 1] International Publication No. WO2018/087625
[Patent Document 2] Japanese Published Patent Application No. 2018-201012

### SUMMARY OF THE INVENTION

As a method for forming an organic semiconductor film in a predetermined shape, a vacuum evaporation method with a metal mask (mask vapor deposition) is widely used. However, in these days of higher density and higher resolution, mask vapor deposition has come close to the limit of increasing the resolution for various reasons such as the alignment accuracy and the distance between the mask and the substrate. By contrast, a finer pattern can be formed by shape processing of an organic semiconductor film by a lithography technique. Moreover, because of the easiness of large-area processing in this method, the processing of an organic semiconductor film by a lithography technique is being researched.

An organic EL device includes an organic compound layer including a light-emitting layer containing a light-emitting substance (corresponding to the above organic semiconductor film) between electrodes (between a first electrode and a second electrode), and energy generated by recombination of carriers (holes and electrons) injected to the organic compound layer from the electrodes causes light emission.

Carrier injection, especially electron injection into the organic compound layer, through which electricity is difficult to flow, has to overcome a high energy barrier and therefore essentially requires a high voltage. In view of this, currently, an electron-injection layer in contact with the cathode includes an alkali metal such as lithium (Li), which has a low work function, or a compound of the alkali metal, whereby a reduction in voltage can be achieved.

However, in the case where the aforementioned lithography method is employed to fabricate a light-emitting device including an organic semiconductor layer using the alkali metal or the compound of the alkali metal described above, oxygen or water in the air and a chemical solution or water used during the processing have caused a significant increase in driving voltage or a marked reduction in current efficiency.

A way of solving the above problem is to perform processing using a lithography method halfway through a process of forming the organic compound layer of a light-emitting device (before forming the layer including an alkali metal or a compound of an alkali metal). In other words, lithography for processing the organic compound layer is performed prior to the formation of the electron-injection layer, and then the formation of the electron-injection layer and the subsequent steps are performed, whereby degradation of characteristics can be avoided.

However, for a tandem light-emitting device, the above solving way cannot be employed and the processing of the organic compound layer by a lithography method has inevitably caused a significant degradation of characteristics.

A tandem light-emitting device includes an organic semiconductor layer in which a plurality of light-emitting layers are stacked in series with an intermediate layer interposed therebetween, and the intermediate layer includes a layer including an alkali metal or a compound of an alkali metal for electron injection into the light-emitting layer on the anode side. Since the intermediate layer is provided between two light-emitting layers, when the organic compound layer including the two light-emitting layers is processed by a lithography method, the intermediate layer is also processed inevitably by a lithography method and is consequently exposed to oxygen, water, and the like.

Thus, like the processing of the electron-injection layer by a lithography method, the processing of the layer including an alkali metal or a compound of an alkali metal in the intermediate layer by a lithography method have caused a significant increase in driving voltage or a marked reduction in current efficiency of the light-emitting device.

Another way of solving the above problem is using an organic compound having an electron-injection property, instead of an alkali metal or a compound of an alkali metal, for the electron-injection layer or the intermediate layer. Specifically, in the above way, the organic compound layer containing neither an alkali metal nor a compound of an alkali metal is processed by a lithography method, so that it is possible to avoid the degradation of light-emitting device characteristics due to an alkali metal or a compound of an alkali metal.

However, if the solubility of the organic compound in water is high, the layer using the organic compound is dissolved in a step of putting the layer in water or a chemical solution containing water as a solvent, which might cause the degradation of characteristics, a defective shape, or the like.

An object of one embodiment of the present invention is to provide an organic compound having an electron-injection property. Another embodiment is to provide an organic compound with low solubility in water. Another embodiment is to provide a light-emitting device with favorable light-emitting characteristics. Another embodiment is to provide a novel organic compound, a novel light-emitting device, a novel light-emitting apparatus, or a novel electronic device.

Note that the description of these objects does not preclude the existence of other objects. One embodiment of the present invention does not need to achieve all of these objects. Other objects can be derived from the description of the specification, the drawings, and the claims.

To solve the above problems, one embodiment of the present invention provides an organic compound including a cyclic guanidine skeleton and an aromatic hydrocarbon skeleton or a heteroaromatic hydrocarbon skeleton. Such an organic compound has an electron-injection property and can be used, instead of an alkali metal or a compound of an alkali metal, for an electron-injection layer or an intermediate layer. Note that the cyclic guanidine skeleton preferably includes an imidazole ring. The cyclic guanidine skeleton including an imidazole ring has low solubility in water and prevents the dissolution of the layer in the processing by a lithography method. Thus, the organic compound of one embodiment of the present invention is used for the electron-injection layer or the intermediate layer instead of an alkali metal or a compound of an alkali metal, whereby the degradation of characteristics due to an alkali metal or a compound of an alkali metal can be avoided and the light-emitting device can have favorable characteristics.

Specifically, one embodiment of the present invention is an organic compound represented by General Formula (G1) below.

### Chemical Formula 1

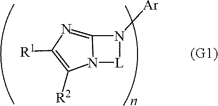

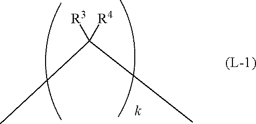

In the organic compound represented by General Formula (G1) above, Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring, each of R¹ and R² independently represents hydrogen (including deuterium), an alkyl group having 1 to 6 carbon atoms, a substituted or unsubstituted amino group, a substituted or unsubstituted aryl group having 6 to 13 carbon atoms forming a ring, or a substituted or unsubstituted heteroaryl group having 2 to 13 carbon atoms forming a ring, n represents an integer greater than or equal to 1 and less than or equal to 6, and L is the group represented by General Formula (L-1) above. In General Formula (L-1) above, each of R³ and R⁴ independently represents hydrogen (including deuterium) or an alkyl group having 1 to 6 carbon atoms, and k is an integer greater than or equal to 1 and less than or equal to 5. When k is greater than or equal to 2, R³s may be the same as or different from each other and R⁴s may be the same as or different from each other.

Another embodiment of the present invention is an organic compound represented by any of General Formulae (G2-1) to (G2-3) below.

### Chemical Formula 2

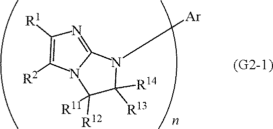

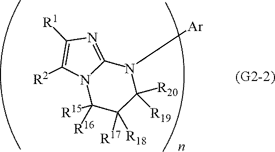

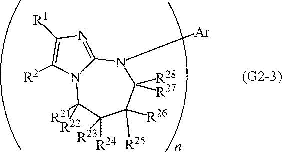

In the organic compound represented by any of General Formulae (G2-1) to (G2-3) above, Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring. Each of R¹, R², and R¹¹ to R²⁸ independently represents hydrogen (including deuterium) or an alkyl group having 1 to 6 carbon atoms, and n represents an integer greater than or equal to 1 and less than or equal to 6.

Another embodiment of the present invention is an organic compound with any of the above structures where each of the aromatic hydrocarbon group having 6 to 30 carbon atoms forming a ring and the heteroaromatic hydrocarbon group having 2 to 30 carbon atoms forming a ring is a group including a structure in which n hydrogen atom(s) is/are removed from any one of rings in any of aromatic hydrocarbons and heteroaromatic hydrocarbons represented by Structural Formulae (Ar-1), (Ar-2), (Ar-3), (Ar-4), (Ar-5), (Ar-6), (Ar-7), (Ar-8), (Ar-9), (Ar-10), (Ar-11), (Ar-12), (Ar-13), (Ar-14), (Ar-15), (Ar-16), (Ar-17), (Ar-18), (Ar-19), (Ar-20), (Ar-21), (Ar-22), (Ar-23), (Ar-24), (Ar-25), (Ar-26), and (Ar-27).

### Chemical Formula 3

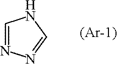

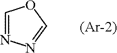

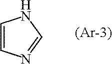

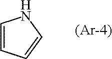

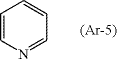

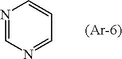

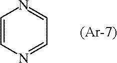

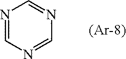

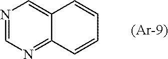

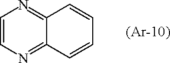

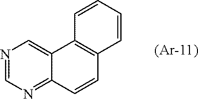 [SMILES: c1ccc2c(c1)ccc1ncncc12]

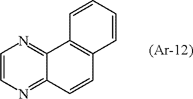 [SMILES: c1ccc2c(c1)ccc1nccnc12]

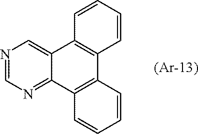 [SMILES: c1ccc2c(c1)c1ccccc1c1ncncc21]

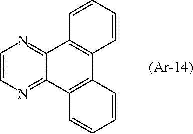 [SMILES: c1ccc2c(c1)c1ccccc1c1nccnc21]

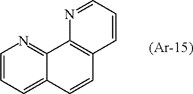 [SMILES: c1cnc2c(c1)ccc1cccnc12]

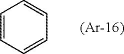

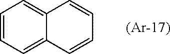

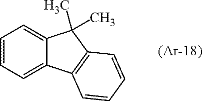

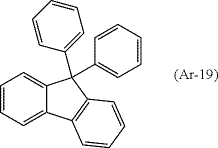 [SMILES: c1ccc(C2(c3ccccc3)c3ccccc3-c3ccccc32)cc1]

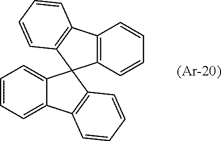 [SMILES: c1ccc2c(c1)-c1ccccc1C21c2ccccc2-c2ccccc21]

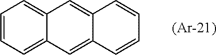 [SMILES: c1ccc2cc3ccccc3cc2c1]

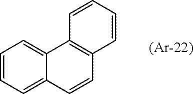 [SMILES: c1ccc2c(c1)ccc1ccccc12]

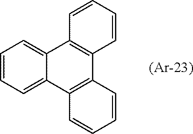 [SMILES: c1ccc2c(c1)c1ccccc1c1ccccc21]

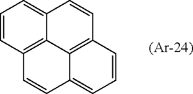 [SMILES: c1cc2ccc3cccc4ccc(c1)c2c34]

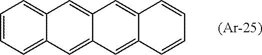 [SMILES: c1ccc2cc3cc4ccccc4cc3cc2c1]

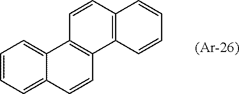 [SMILES: c1ccc2c(c1)ccc1c3ccccc3ccc21]

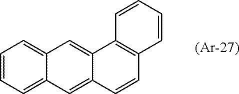 [SMILES: c1ccc2cc3c(ccc4ccccc43)cc2c1]

Another embodiment of the present invention is an organic compound represented by Structural Formula (100), (101), or (113) below.

### Chemical Formula 4

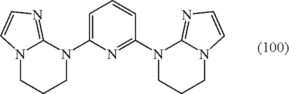 [SMILES: c1cc(N2CCCn3ccnc32)nc(N2CCCn3ccnc32)c1]

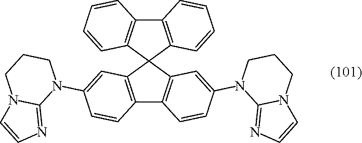 [SMILES: c1ccc2c(c1)-c1ccccc1C21c2cc(N3CCCn4ccnc43)ccc2-c2ccc(N3CCCn4ccnc43)cc21]

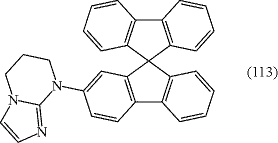 [SMILES: c1ccc2c(c1)-c1ccccc1C21c2ccccc2-c2ccc(N3CCCn4ccnc43)cc21]

Another embodiment of the present invention is a light-emitting device including the organic compound having any of the above structures.

Another embodiment of the present invention is a light-emitting apparatus including the light-emitting device having the above structure, and at least one of a transistor and a substrate.

Another embodiment of the present invention is an electronic device including the above light-emitting apparatus; and a sensor unit, an input unit, or a communication unit.

Note that the light-emitting apparatus in this specification includes, in its category, an image display device that uses a light-emitting device. The light-emitting apparatus may also include a module in which a light-emitting device over a substrate is provided with a connector such as an anisotropic conductive film or a tape carrier package (TCP), a module in which a printed wiring board is provided at the end of a TCP, and a module in which an integrated circuit (IC) is directly mounted on a light-emitting device by a chip on glass (COG) method. Furthermore, a lighting device or the like may include the light-emitting apparatus.

One embodiment of the present invention can provide an organic compound having an electron-injection property. Another embodiment can provide an organic compound with low solubility in water. Another embodiment can provide a light-emitting device with favorable light-emitting characteristics. Another embodiment can provide a novel organic compound or a novel light-emitting device.

One embodiment of the present invention can provide a light-emitting apparatus with high display quality. Another embodiment can provide a high-resolution light-emitting apparatus. Another embodiment can provide a high-definition light-emitting apparatus. Another embodiment can provide a highly reliable light-emitting apparatus. Another embodiment can provide a novel light-emitting apparatus that is highly convenient, useful, or reliable. Another embodiment can provide a novel display module that is highly convenient, useful, or reliable. Another embodiment can provide a novel electronic device that is highly convenient, useful, or reliable. Another embodiment can provide a novel light-emitting apparatus, a novel display module, a novel electronic device, or a novel semiconductor device.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIGS. 1A to 1C each illustrate a light-emitting device.

FIG. 2 illustrates a light-emitting device.

FIGS. 3A and 3B are, respectively, a top view and a cross-sectional view of a light-emitting apparatus.

FIGS. 4A to 4D each illustrate a light-emitting device.

FIGS. 5A to 5E are cross-sectional views illustrating an example of a method of manufacturing a display apparatus.

FIGS. 6A to 6D are cross-sectional views illustrating an example of a method of manufacturing a display apparatus.

FIGS. 7A to 7D are cross-sectional views illustrating an example of a method of manufacturing a display apparatus.

FIGS. 8A to 8C are cross-sectional views illustrating an example of a method of manufacturing a display apparatus.

FIGS. 9A to 9C are cross-sectional views illustrating an example of a method of manufacturing a display apparatus.

FIGS. 10A to 10C are cross-sectional views illustrating an example of a method of manufacturing a display apparatus.

FIGS. 11A and 11B are perspective views illustrating a structure example of a display module.

FIGS. 12A and 12B are cross-sectional views illustrating structure examples of a display apparatus.

FIGS. 13A to 13D illustrate examples of an electronic device.

FIGS. 14A to 14F illustrate examples of electronic devices.

FIGS. 15A to 15C show a ¹H NMR spectrum of 2,6tip2Py.

FIG. 16 shows an absorption spectrum and an emission spectrum of 2,6tip2Py in a toluene solution.

FIGS. 17A to 17C show a ¹H NMR spectrum of 2,7tip2SF.

FIG. 18 shows an absorption spectrum and an emission spectrum of 2,7tip2SF in a toluene solution.

FIG. 19 shows the luminance-current density characteristics of Light-emitting device 1.

FIG. 20 shows the current efficiency-luminance characteristics of Light-emitting device 1.

FIG. 21 shows the luminance-voltage characteristics of Light-emitting device 1.

FIG. 22 shows the current-voltage characteristics of Light-emitting device 1.

FIG. 23 shows the electroluminescence spectrum of Light-emitting device 1.

FIG. 24 shows the luminance-current density characteristics of Light-emitting device 2.

FIG. 25 shows the current efficiency-luminance characteristics of Light-emitting device 2.

FIG. 26 shows the luminance-voltage characteristics of Light-emitting device 2.

FIG. 27 shows the current-voltage characteristics of Light-emitting device 2.

FIG. 28 shows the electroluminescence spectrum of Light-emitting device 2.

FIG. 29 shows a driving time-dependent change in luminance of Light-emitting device 2.

FIG. 30 shows the luminance-current density characteristics of Light-emitting device 3.

FIG. 31 shows the current efficiency-luminance characteristics of Light-emitting device 3.

FIG. 32 shows the luminance-voltage characteristics of Light-emitting device 3.

FIG. 33 shows the current-voltage characteristics of Light-emitting device 3.

FIG. 34 shows the electroluminescence spectrum of Light-emitting device 3.

FIG. 35 shows the luminance-current density characteristics of Light-emitting device 4.

FIG. 36 shows the current efficiency-luminance characteristics of Light-emitting device 4.

FIG. 37 shows the luminance-voltage characteristics of Light-emitting device 4.

FIG. 38 shows the current-voltage characteristics of Light-emitting device 4.

FIG. 39 shows the electroluminescence spectrum of Light-emitting device 4.

FIG. 40 shows a driving time-dependent change in luminance of Light-emitting device 4.

FIGS. 41A to 41C show a ¹H NMR spectrum of tipSF.

FIG. 42 is a molecular structure image obtained by X-ray crystallography.

### DETAILED DESCRIPTION OF THE INVENTION

Embodiments will be described in detail with reference to the drawings. Note that the embodiments of the present invention are not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

Note that in structures of the invention described below, the same portions or portions having similar functions are denoted by the same reference numerals in different drawings, and the description thereof is not repeated. The same hatching pattern is used for portions having similar functions, and the portions are not denoted by specific reference numerals in some cases.

The position, size, range, or the like of each component illustrated in drawings does not represent the actual position, size, range, or the like in some cases for easy understanding. Therefore, the disclosed invention is not necessarily limited to the position, size, range, or the like disclosed in the drawings.

Note that the terms “film” and “layer” can be used interchangeably depending on the case or the circumstances. For example, the term “conductive layer” can be replaced with the term “conductive film.” As another example, the term “insulating film” can be replaced with the term “insulating layer.”

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure.

In this specification and the like, a hole or an electron is sometimes referred to as a carrier. Specifically, a hole-injection layer or an electron-injection layer may be referred to as a carrier-injection layer, a hole-transport layer or an electron-transport layer may be referred to as a carrier-transport layer, and a hole-blocking layer or an electron-blocking layer may be referred to as a carrier-blocking layer. Note that the above-described carrier-injection layer, carrier-transport layer, and carrier-blocking layer cannot be distinguished from each other depending on the cross-sectional shape or properties in some cases. One layer may have two or three functions of the carrier-injection layer, the carrier-transport layer, and the carrier-blocking layer in some cases.

In this specification and the like, a light-emitting device (also referred to as a light-emitting element) includes an EL layer between a pair of electrodes. The EL layer includes at least a light-emitting layer. In this specification and the like, a light-receiving device (also referred to as a light-receiving element) includes at least an active layer functioning as a photoelectric conversion layer between a pair of electrodes. In this specification and the like, one of the pair of electrodes may be referred to as a pixel electrode and the other may be referred to as a common electrode.

In this specification and the like, a tapered shape indicates a shape in which at least part of a side surface of a structure is inclined to a substrate surface. For example, a tapered shape preferably includes a region where the angle between the inclined side surface and the substrate surface (such an angle is also referred to as a taper angle) is less than 90°. Note that the side surface of the component and the substrate surface is not necessarily completely flat, and may have a substantially planar shape with a small curvature or slight unevenness.

Note that the light-emitting apparatus in this specification includes, in its category, an image display devices that uses an organic EL device. The light-emitting apparatus may also include a module in which an organic EL device is provided with a connector such as an anisotropic conductive film or a tape carrier package (TCP), a module in which a printed wiring board is provided at the end of a TCP, and a module in which an integrated circuit (IC) is directly mounted on an organic EL device by a chip on glass (COG) method. Furthermore, a lighting device or the like may include the light-emitting apparatus.

### Embodiment 1

In this embodiment, an organic compound of one embodiment of the present invention is described.

As described above, to solve the above problems, one embodiment of the present invention provides an organic compound including a cyclic guanidine skeleton and an aromatic hydrocarbon skeleton or a heteroaromatic hydrocarbon skeleton. Such an organic compound has an electron-injection property and can be used, instead of an alkali metal or a compound of an alkali metal, for an electron-injection layer or an intermediate layer. Note that the cyclic guanidine skeleton preferably includes an imidazole ring. The cyclic guanidine skeleton including an imidazole ring has low solubility in water and prevents the dissolution of the layer in the processing by a lithography method. Thus, the organic compound of one embodiment of the present invention is used for the electron-inj ection layer or the intermediate layer instead of an alkali metal or a compound of an alkali metal, whereby the degradation of characteristics due to an alkali metal or a compound of an alkali metal can be avoided and the light-emitting device can have favorable characteristics.

Specifically, one embodiment of the present invention is an organic compound represented by General Formula (G1) below.

### [Chemical Formula 5]

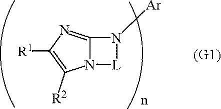

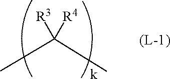

In the organic compound represented by General Formula (G1) above, Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring, each of R¹ and R² independently represents hydrogen (including deuterium), an alkyl group having 1 to 6 carbon atoms, a substituted or unsubstituted amino group, a substituted or unsubstituted aryl group having 6 to 13 carbon atoms forming a ring, or a substituted or unsubstituted heteroaryl group having 2 to 13 carbon atoms forming a ring, n represents an integer greater than or equal to 1 and less than or equal to 6, and L is the group represented by General Formula (L-1) above. In General Formula (L-1) above, each of R³ and R⁴ independently represents hydrogen (including deuterium) or an alkyl group having 1 to 6 carbon atoms, and k is an integer greater than or equal to 1 and less than or equal to 5. When k is greater than or equal to 2, R³s may be the same as or different from each other and R⁴s may be the same as or different from each other.

As described above, the structure including the cyclic guanidine skeleton enables the organic compound to have an electron-injection property. In addition, when the cyclic guanidine skeleton includes an imidazole ring, solubility in water can be reduced. Accordingly, the light-emitting device using the organic compound with this structure in the electron-injection layer or the intermediate layer can have favorable characteristics.

Another embodiment of the present invention is an organic compound represented by any of General Formulae (G2-1) to (G2-3) below.

### [Chemical Formula 6]

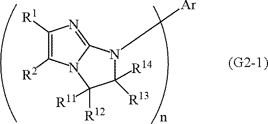

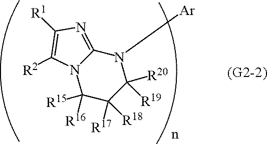

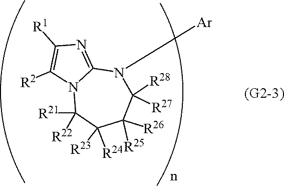

In the organic compound represented by any of General Formulae (G2-1) to (G2-3) above, Ar represents a substituted or unsubstituted aromatic hydrocarbon group having 6 to 30carbon atoms forming a ring or a substituted or unsubstituted heteroaromatic hydrocarbon group having 2 to 30 carbon atoms forming a ring. Each of R¹, R², and R¹¹ to R²⁸ independently represents hydrogen (including deuterium) or an alkyl group having 1 to 6 carbon atoms, and n represents an integer greater than or equal to 1 and less than or equal to 6.

The organic compounds represented by General Formulae (G2-1) to (G2-3) above each have a structure in which k in General Formula (G1) above is limited to an integer greater than or equal to 2 and less than or equal to 4. This structure is preferred because the stability of the cyclic guanidine skeleton can be increased and accordingly the stability of the organic compound as a whole can be increased.

In each of General Formulae (G1) and (G2-1) to (G2-3) above, n is preferably an integer greater than or equal to 1 and less than or equal to 4, further preferably 1 or 2. With such a structure, the solubility of the organic compound in water or a chemical solution containing water as a solvent can be low.

### Specific Examples of Aromatic Hydrocarbons and Heteroaromatic Hydrocarbons

In each of the organic compounds represented by General Formulae (G1) and (G2-1) to (G2-3), the aromatic hydrocarbon group having 6 to 30 carbon atoms forming a ring is a group having a structure in which n hydrogen atom(s) is/are removed from an aromatic hydrocarbon having 6 to 30 carbon atoms forming a ring, and the heteroaromatic hydrocarbon group having 2 to 30 carbon atoms forming a ring is a group having a structure in which n hydrogen atom(s) is/are removed from a heteroaromatic hydrocarbon having 2 to 30 carbon atoms forming a ring.

Specific examples of the aromatic hydrocarbon having 6 to 30 carbon atoms forming a ring, which can be used as the aromatic hydrocarbon group having 6 to 30 carbon atoms forming a ring when n hydrogen atom(s) is/are removed in the organic compound represented by any of General Formulae (G1) and (G2-1) to (G2-3) above, include benzene, naphthalene, fluorene, spirobifluorene, anthracene, phenanthrene, triphenylene, pyrene, tetracene, chrysene, and benz(a)anthracene. Note that specific examples of the aromatic hydrocarbon group having 6 to 30 carbon atoms forming a ring are not limited to these.

Specific examples of the heteroaromatic hydrocarbon having 2 to 30 carbon atoms forming a ring, which can be used as the heteroaromatic hydrocarbon group having 2 to 30 carbon atoms forming a ring when n hydrogen atom(s) is/are removed, in the organic compound represented by any of General Formulae (G1) and (G2-1) to (G2-3) above, include pyridine, bipyridine, pyrimidine, bipyrimidine, pyrazine, bipyrazine, triazine, quinoline, isoquinoline, benzoquinoline, phenanthroline, quinoxaline, benzoquinoxaline, dibenzoquinoxaline, azafluorene, diazafluorene, carbazole, benzocarbazole, dibenzocarbazole, dibenzofuran, benzonaphthofuran, dinaphthofuran, dibenzothiophene, benzonaphthothiophene, dinaphthothiophene, benzofuropyridine, benzofuropyrimidine, benzothiopyridine, benzothiopyrimidine, naphthofuropyridine, naphthofuropyrimidine, naphthothiopyridine, naphthothiopyrimidine, acridine, xanthene, phenothiazine, phenoxazine, phenazine, triazole, oxazole, oxadiazole, thiadiazole, imidazole, benzimidazole, pyrazole, and pyrrole. Note that specific examples of the heteroaromatic hydrocarbon group having 2 to 30 carbon atoms forming a ring are not limited to these.

Any one of organic compounds represented by Structural Formulae (Ar-1) to (Ar-27) below is preferred as further specific examples of the above aromatic hydrocarbon having 6 to 30 carbon atoms forming a ring and the heteroaromatic hydrocarbon having 2 to 30 carbon atoms forming a ring which can be used as the aromatic hydrocarbon group having 6 to 30 carbon atoms forming a ring or the heteroaromatic hydrocarbon group having 2 to 30 carbon atoms forming a ring when n hydrogen atom(s) is/are removed in the organic compound represented by any of General Formulae (G1) and (G2-1) to (G2-3) above.

### [Chemical Formula 7]

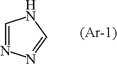

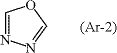

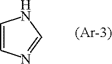

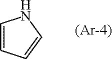

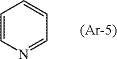

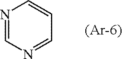

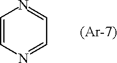

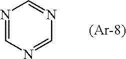

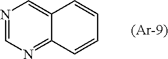

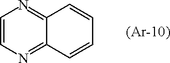

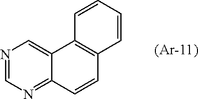 [SMILES: c1ccc2c(c1)ccc1ncncc12]

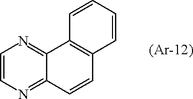 [SMILES: c1ccc2c(c1)ccc1nccnc12]

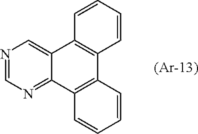 [SMILES: c1ccc2c(c1)c1ccccc1c1ncncc21]

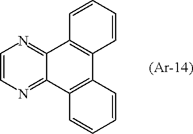 [SMILES: c1ccc2c(c1)c1ccccc1c1nccnc21]

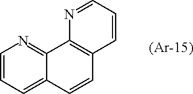 [SMILES: c1cnc2c(c1)ccc1cccnc12]

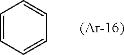

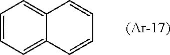

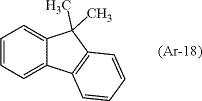

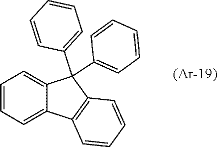 [SMILES: c1ccc(C2(c3ccccc3)c3ccccc3-c3ccccc32)cc1]

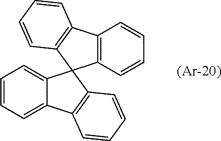 [SMILES: c1ccc2c(c1)-c1ccccc1C21c2ccccc2-c2ccccc21]

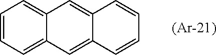 [SMILES: c1ccc2cc3ccccc3cc2c1]

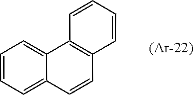 [SMILES: c1ccc2c(c1)ccc1ccccc12]

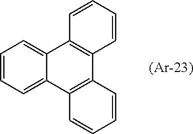 [SMILES: c1ccc2c(c1)c1ccccc1c1ccccc21]

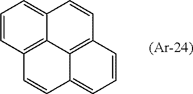 [SMILES: c1cc2ccc3cccc4ccc(c1)c2c34]

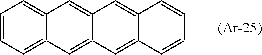 [SMILES: c1ccc2cc3cc4ccccc4cc3cc2c1]

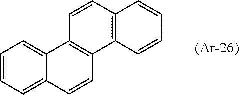 [SMILES: c1ccc2c(c1)ccc1c3ccccc3ccc21]

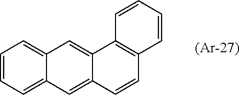 [SMILES: c1ccc2cc3c(ccc4ccccc43)cc2c1]

In any of the above aromatic hydrocarbon having 6 to 30 carbon atoms forming a ring and the above heteroaromatic hydrocarbon having 2 to 30 carbon atoms forming a ring, the group having the structure in which n hydrogen atom(s) is/are removed from Structural Formula (Ar-5) or (Ar-20) is further preferably used as Ar. With the use of such an aromatic hydrocarbon or heteroaromatic hydrocarbon, the solubility in water or a chemical solution containing water as a solvent can be low.

When the heteroaromatic hydrocarbon having 2 to 30 carbon atoms forming a ring includes nitrogen as an element forming the ring, the nitrogen or carbon adjacent to the nitrogen is preferably bonded to the cyclic guanidine skeleton. This can increase the electron-injection property of the organic compound.

When the aromatic hydrocarbon group having 6 to 30 carbon atoms forming a ring or a substituted or unsubstituted heteroaromatic hydrocarbon group having 2 to 30 carbon atoms forming a ring has a substituent, specific examples of the substituent include an alkyl group having 1 to 6 carbon atoms, an aryl group having 6 to 13 carbon atoms, and a heteroaryl group having 2 to 13 carbon atoms. Some or all of the hydrogen atoms included in the aromatic hydrocarbon group having 6 to 30 carbon atoms forming a ring or the substituted or unsubstituted heteroaromatic hydrocarbon group having 2 to 30 carbon atoms forming a ring may be deuterium.

### Specific Examples of Alkyl Group Having 1 to 6 Carbon Atoms

Specific examples of the alkyl group having 1 to 6 carbon atoms in the organic compound represented by any of General Formulae (G1) and (G2-1) to (G2-3) above include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, and a 2,3-dimethylbutyl group. Note that some or all of hydrogen atoms included in the alkyl group having 1 to 6 carbon atoms may be deuterium.

### Specific Examples of Substituted or Unsubstituted Amino Group

Specific examples of the substituted or unsubstituted amino group in the organic compound represented by any of General Formulae (G1) and (G2-1) to (G2-3) above include -NH₂, a dialkylamino group, and a diarylamino group. Specific examples of the alkyl group that can be used as a dialkylamino group include an alkyl group having 1 to 6 carbon atoms. Specific examples of the aryl group that can be used as a diarylamino group include an aryl having group 6 to 13 carbon atoms forming a ring. Note that some or all of the hydrogen atoms included in the substituted or unsubstituted amino group may be deuterium.

### Specific Examples of Aryl Having Group 6 to 13 Carbon Atoms Forming Ring

Specific examples of the aryl having group 6 to 13 carbon atoms forming a ring in the organic compound represented by any of General Formulae (G1) and (G2-1) to (G2-3) above include a phenyl group, an o-tolyl group, an m-tolyl group, a p-tolyl group, a mesityl group, an o-biphenyl group, an m-biphenyl group, a p-biphenyl group, a 1-naphthyl group, a 2-naphthyl group, and a fluorenyl group. In the case where the aryl having group 6 to 13 carbon atoms forming a ring has a substituent, specific examples of the substituent include an alkyl group having 1 to 6 carbon atoms, an aryl group having 6 to 13 carbon atoms forming a ring, and a heteroaryl group having 2 to 13 carbon atoms forming a ring. Some or all of hydrogen atoms included in the aryl group having 6 to 13 carbon atoms may be deuterium.

### Specific Examples of Heteroaryl Having Group 2 to 13 Carbon Atoms Forming Ring

Specific examples of the heteroaryl having group 2 to 13 carbon atoms forming a ring in the organic compound represented by any of General Formulae (G1) and (G2-1) to (G2-3) above include an imidazolyl group, a pyrazolyl group, a pyridyl group, a pyridazyl group, a triazyl group, a benzimidazolyl group, a quinolyl group, a carbazolyl group, a dibenzofuranyl group, and a dibenzothiophenyl group. In the case where the heteroaryl having group 2 to 13 carbon atoms forming a ring has a substituent, specific examples of the substituent include an alkyl group having 1 to 6 carbon atoms, an aryl group having 6 to 13 carbon atoms forming a ring, and a heteroaryl group having 2 to 13 carbon atoms forming a ring. Some or all of hydrogen atoms included in the heteroaryl having group 2 to 13 carbon atoms forming a ring may be deuterium.

Specific examples of the organic compounds represented by General Formulae (G1) and (G2-1) to (G2-3) above include organic compounds represented by Structural Formulae (100) to (114) below.

### [Chemical Formula 8]

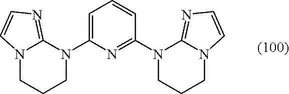 [SMILES: c1cc(N2CCCn3ccnc32)nc(N2CCCn3ccnc32)c1]

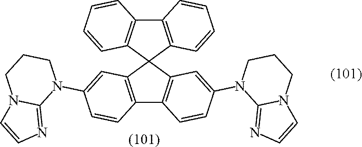

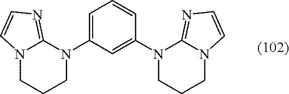 [SMILES: c1cc(N2CCCn3ccnc32)cc(N2CCCn3ccnc32)c1]

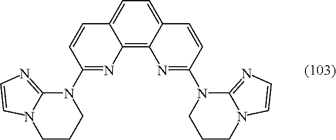 [SMILES: c1cn2c(n1)N(c1ccc3ccc4ccc(N5CCCn6ccnc65)nc4c3n1)CCC2]

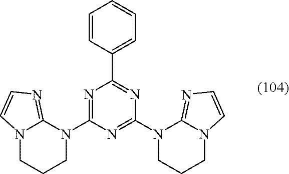 [SMILES: c1ccc(-c2nc(N3CCCn4ccnc43)nc(N3CCCn4ccnc43)n2)cc1]

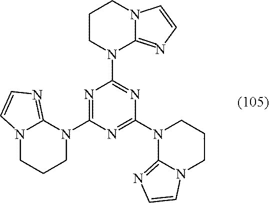 [SMILES: c1cn2c(n1)N(c1nc(N3CCCn4ccnc43)nc(N3CCCn4ccnc43)n1)CCC2]

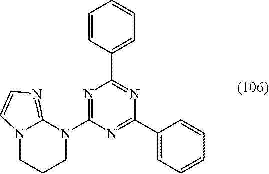 [SMILES: c1ccc(-c2nc(-c3ccccc3)nc(N3CCCn4ccnc43)n2)cc1]

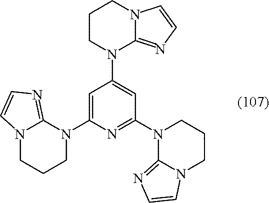 [SMILES: c1cn2c(n1)N(c1cc(N3CCCn4ccnc43)nc(N3CCCn4ccnc43)c1)CCC2]

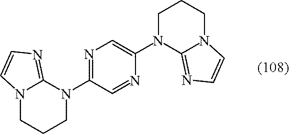 [SMILES: c1cn2c(n1)N(c1cnc(N3CCCn4ccnc43)cn1)CCC2]

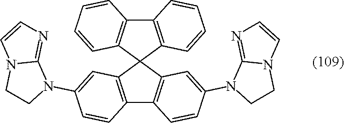 [SMILES: c1ccc2c(c1)-c1ccccc1C21c2cc(N3CCn4ccnc43)ccc2-c2ccc(N3CCn4ccnc43)cc21]

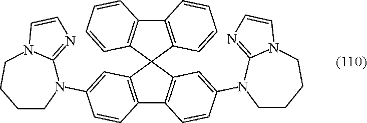 [SMILES: c1ccc2c(c1)-c1ccccc1C21c2cc(N3CCCCn4ccnc43)ccc2-c2ccc(N3CCCCn4ccnc43)cc21]

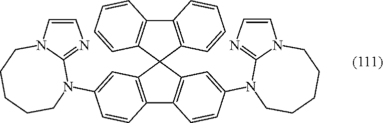 [SMILES: c1ccc2c(c1)-c1ccccc1C21c2cc(N3CCCCCn4ccnc43)ccc2-c2ccc(N3CCCCCn4ccnc43)cc21]

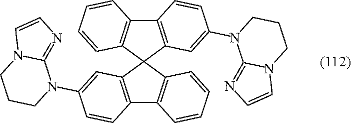 [SMILES: c1ccc2c(c1)-c1ccc(N3CCCn4ccnc43)cc1C21c2ccccc2-c2ccc(N3CCCn4ccnc43)cc21]

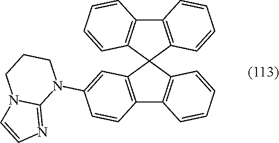 [SMILES: c1ccc2c(c1)-c1ccccc1C21c2ccccc2-c2ccc(N3CCCn4ccnc43)cc21]

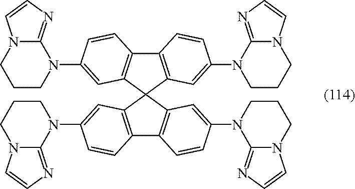 [SMILES: c1cn2c(n1)N(c1ccc3c(c1)C1(c4cc(N5CCCn6ccnc65)ccc4-3)c3cc(N4CCCn5ccnc54)ccc3-c3ccc(N4CCCn5ccnc54)cc31)CCC2]

The organic compounds represented by Structural Formulae (100) to (114) are examples of the organic compounds represented by General Formulae ((G1) and (G2-1) to (G2-3) above. The organic compound of one embodiment of the present invention is not limited thereto.

Next, as an example of a method of synthesizing the organic compound of one embodiment of the present invention, a method of synthesizing the organic compound represented by General Formula (G1) below is described. Note that the synthesis method of the organic compound represented by General Formula (G1) can employ a variety of reactions and is not limited to the following synthesis methods.

### [Chemical Formula 9]

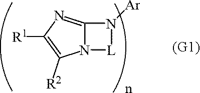

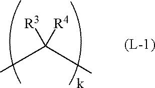

In the organic compound represented by General Formula (G1) above, Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring, each of R¹ and R² independently represents hydrogen (including deuterium), an alkyl group having 1 to 6 carbon atoms, a substituted or unsubstituted amino group, a substituted or unsubstituted aryl group having 6 to 13 carbon atoms forming a ring, or a substituted or unsubstituted heteroaryl group having 2 to 13 carbon atoms forming a ring, n represents an integer greater than or equal to 1 and less than or equal to 6, and L is the group represented by General Formula (L-1) above. In General Formula (L-1) above, each of R³ and R⁴ independently represents hydrogen (including deuterium) or an alkyl group having 1 to 6 carbon atoms, and k is an integer greater than or equal to 1 and less than or equal to 5. When k is greater than or equal to 2, R³s may be the same as or different from each other and R⁴s may be the same as or different from each other.

### <<Method of Synthesizing Organic Compound Represented by General Formula (G1)>>

The organic compound of one embodiment of the present invention represented by General Formula (G1) can be synthesized by Synthesis Scheme (A-1) shown below. Specifically, the organic compound of one embodiment of the present invention represented by General Formula (G1) can be obtained by coupling of an organic compound represented by General Formula (a1), which is either a halogen compound of the aromatic hydrocarbon or the heteroaromatic hydrocarbon or the compound having a triflate group bonded to the aromatic hydrocarbon or the heteroaromatic hydrocarbon, with an organic compound represented by General Formula (b1), which includes a secondary amino group, through the Buchwald-Hartwig reaction, for example.

### [Chemical Formula 10]

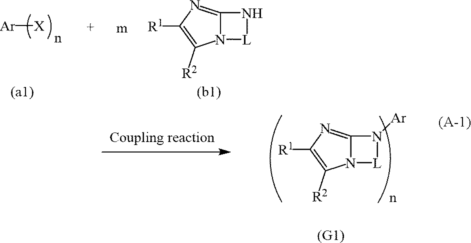

In Synthesis Scheme (A-1) above, L is a group represented by General Formula (L-1) below.

### [Chemical Formula 11]

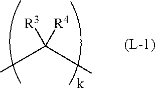

In General Formula (a1) above, Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring, n represents an integer greater than or equal to 1 and less than or equal to 6, and X represents a halogen or a triflate group, preferably chlorine, bromine, or iodine, in particular.

In General Formula (b1) above, each of R¹ and R² independently represents hydrogen (including deuterium), an alkyl group having 1 to 6 carbon atoms, a substituted or unsubstituted amino group, a substituted or unsubstituted aryl group having 6 to 13 carbon atoms forming a ring, or a substituted or unsubstituted heteroaryl group having 2 to 13 carbon atoms forming a ring, and L is the group represented by General Formula (L-1) above. In General Formula (L-1) above, each of R³ and R⁴ independently represents hydrogen (including deuterium) or an alkyl group having 1 to 6 carbon atoms, and k is an integer greater than or equal to 1 and less than or equal to 5. When k is greater than or equal to 2, R³s may be the same as or different from each other and R⁴s may be the same as or different from each other. In Synthesis scheme (A-1) above, preferably, m is a positive number and is greater than n.

Specific examples of the palladium catalyst that can be used for the coupling reaction represented by Synthesis Scheme (A-1) above include palladium(II) acetate, tetrakis(triphenylphosphine)palladium(0), and bis(triphenylphosphine)palladium(II) dichloride. Specific examples of the ligand that can be used in the above palladium catalyst include (±)-2,2′-bis(diphenylphosphino)-1,1′-binaphthyl, tri(ortho-tolyl)phosphine, triphenylphosphine, and tricyclohexylphosphine.

Specific examples of the base that can be used for the coupling reaction represented by Synthesis Scheme (A-1) above include an organic base such as potassium tert-butoxide and an inorganic base such as potassium carbonate or sodium carbonate.

Specific examples of the solvent that can be used for the coupling reaction represented by Synthesis Scheme (A-1) above include toluene, xylene, mesitylene, benzene, tetrahydrofuran, and dioxane. However, the solvent that can be used is not limited to these solvents.

The reaction employed in Synthesis Scheme (A-1) is not limited to the Buchwald-Hartwig reaction. A Migita-Kosugi-Stille coupling reaction using an organotin compound, a coupling reaction using a Grignard reagent, an Ullmann reaction using copper or a copper compound, a nucleophilic substitution reaction, or the like can be used.

The method of synthesizing the organic compound represented by General Formula (G1) is not limited to the above described method.

The structures described in this embodiment can be used in combination with any of the structures described in the other embodiments as appropriate.

### Embodiment 2

In this embodiment, a structure of a light-emitting device using any of the organic compounds of one embodiment of the present invention is described.

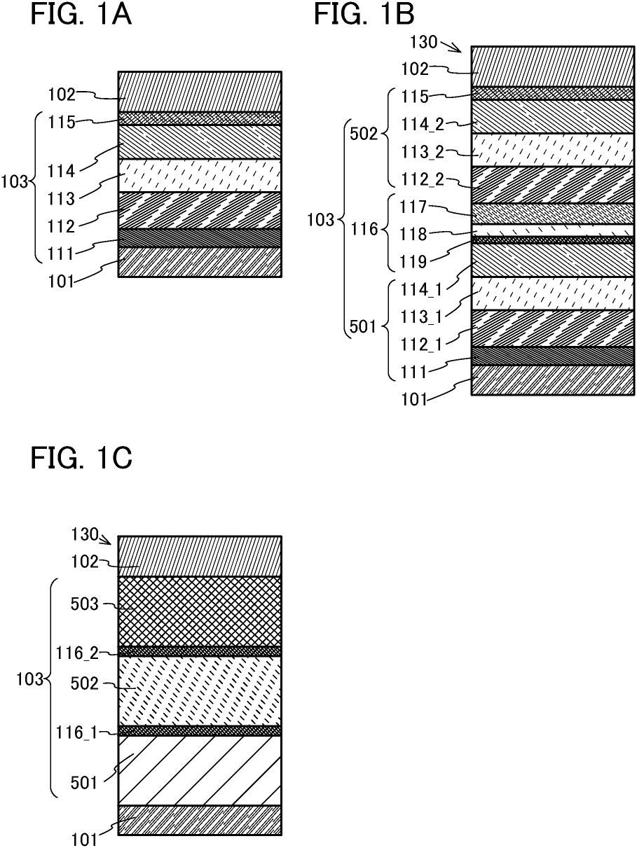

FIG. 1A illustrates a light-emitting device 130, which is an example of the light-emitting device of one embodiment of the present invention. The light-emitting device 130 includes an organic compound layer 103 including a light-emitting layer 113 between a first electrode 101 including an anode and a second electrode 102 including a cathode.

FIG. 1B illustrates another light-emitting device 130 that is a different example of the light-emitting device of one embodiment of the present invention. The light-emitting device 130 is a tandem light-emitting device. The light-emitting device 130 includes a first light-emitting unit 501 including a first light-emitting layer 113_1, a second light-emitting unit 502 including a second light-emitting layer 113_2, and an intermediate layer 116, as the organic compound layer 103.

Although a light-emitting device including one intermediate layer 116 and two light-emitting units is described as an example in this embodiment, a light-emitting device including n charge generation layer(s) (n is an integer greater than or equal to 1) and n+1 light-emitting units may be employed.

For example, the light-emitting device 130 illustrated in FIG. 1C is an example of the tandem light-emitting device where n is 2, including the first light-emitting unit 501, a first intermediate layer 116_1, the second light-emitting unit 502, a second intermediate layer 116_2, and a third light-emitting unit 503, as the organic compound layer 103. Note that the color gamut of light exhibited by the light-emitting layers in the light-emitting units may be the same or different. In addition, the light-emitting layer may have a single-layer structure or a stacked structure. For example, the first and third light-emitting units exhibit light in a blue region while the second light-emitting unit exhibits light in a red region and light in a green region including stacked light-emitting layers, whereby white light emission can be obtained.

The light-emitting device 130 may be fabricated by a photolithography method, for example. In the case of the light-emitting device fabricated by a photolithography method, at least the light-emitting layer 113 (or the second light-emitting layer 113_2) and the layer(s) in the organic compound layer that is/are closer to the first electrode 101 than the light-emitting layer are processed at the same time; consequently, their end portions are substantially aligned in the perpendicular direction.

The organic compound layer 103 may include another functional layer in addition to the light-emitting layer. FIG. 1A shows a structure in which, in addition to the light-emitting layer 113, a hole-injection layer 111, a hole-transport layer 112, an electron-transport layer 114, and an electron-injection layer 115 are provided in the organic compound layer 103. Furthermore, the first light-emitting unit 501 and the second light-emitting unit 502 may include another functional layer in addition to the light-emitting layer. FIG. 1B shows a structure in which the hole-injection layer 111, a first hole-transport layer 112_1, and a first electron-transport layer 114_1, in addition to the first light-emitting layer 113_1, are provided in the first light-emitting unit 501 and a second hole-transport layer 112_2, a second electron-transport layer 114_2, and the electron-injection layer 115, in addition to the second light-emitting layer 113_2, are provided in the second light-emitting unit 502. The structure of the organic compound layer 103 in the present invention is not limited to these structures; any of the layers may be absent or another layer may be added. A carrier-blocking layer (a hole-blocking layer or an electron-blocking layer), an exciton-blocking layer, or the like may be typically added.

### <<Structure of Intermediate Layer>>

First, a material that can be used for the intermediate layer 116 is described. Any of the organic compounds of one embodiment of the present invention described in Embodiment 1 can be used for the intermediate layer 116. Specifically, the intermediate layer 116 is a layer including a first layer 119 and a second layer 117, and any of the organic compounds of one embodiment of the present invention described in Embodiment 1 is preferably used for the first layer 119.

The second layer 117 is positioned closer to the second electrode 102 than the first layer 119 is. Between the first layer 119 and the second layer 117, a third layer 118 for smoothing electron transfer between the two layers may be provided.

Since the first layer 119 is included in the intermediate layer 116, the first layer 119 serves as an electron-injection layer in the light-emitting unit closer to the anode. Thus, an electron-injection layer is not necessarily provided in the light-emitting unit on the anode side (the first light-emitting unit 501 in FIG. 1B). Similarly, since the second layer 117 is included in the intermediate layer 116, the second layer 117 serves as a hole-injection layer in the light-emitting unit closer to the cathode. Thus, a hole-injection is not necessarily provided in the light-emitting unit on the cathode side (the second light-emitting unit 502 in FIG. 1B).

The first layer 119 may include an organic compound having an electron-transport property in addition to any of the organic compounds of one embodiment of the present invention.

The organic compound having an electron-transport property that can be used for the first layer 119 is preferably a substance with an electron mobility higher than or equal to 1 × 10⁻⁷ cm²/Vs, preferably higher than or equal to 1 × 10⁻⁶ cm²/Vs, when the square root of electric field strength [V/cm] is 600. Note that any other substance can be used as long as the substance has an electron-transport property higher than a hole-transport property.

An organic compound including a π-electron deficient heteroaromatic ring is preferable as the above organic compound. The organic compound including a π-electron deficient heteroaromatic ring is preferably one or more of an organic compound including a heteroaromatic ring having a polyazole skeleton, an organic compound including a heteroaromatic ring having a pyridine skeleton, an organic compound including a heteroaromatic ring having a diazine skeleton, and an organic compound including a heteroaromatic ring having a triazine skeleton.

Specific examples of the organic compound having an electron-transport property that can be used for the first layer 119 include organic compounds having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs); organic compounds having a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: BPhen), bathocuproine (abbreviation: BCP), 2,9-di(naphthalen-2-yl)-4,7-diphenyl-1,10-phenanthroline (abbreviation: NBPhen), and 2,2′-(1,3-phenylene)bis(9-phenyl-1,10-phenanthroline) (abbreviation: mPPhen2P); organic compounds having a diazine skeleton, such as 2-[3-(dibenzothiophen-4-yl)phenyl]dibenzo[f,h]quinoxaline (abbreviation: 2mDBTPDBq-II), 2-[3-(3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[(3′-dibenzothiophen-4-yl)biphenyl-3-yl]naphtho[1′,2′:4,5]furo[2,3-b]pyrazine (abbreviation: 9mDBtBPNfpr), 9-[3′-dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(4-dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl) (1,1′-biphenyl-3-yl)]naphtho[1′,2′:4,5]furo[3,2-d]pyrimidine (abbreviation: 8mDBtBPNfpm), 8-[(2,2′-binaphthalen)-6-yl)]-4-[3-(dibenzothiophen-4-yl)phenyl]-[1]benzofuro[3,2-d]pyrimidine (abbreviation: 8(βN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis{4-[4-(2-naphthyl)phenyl]-6-phenylpyrimidine} (abbreviation: 2,6(NP-PPm)2Py), 6-(1,1′-biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(1,1′-biphenyl-4-yl)pyrimidine (abbreviation: 6BP-4Cz2PPm), and 7-[4-(9-phenyl-9H-carbazol-2-yl)quinazolin-2-yl]-7H-dibenzo[c,g]carbazol (abbreviation: PC-cgDBCzQz); and organic compounds having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)-1,1′-biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2-[3-(2,6-dimethyl-3-pyridinyl)-5-(9-phenanthrenyl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-α]carbazole (abbreviation: BP-Icz(II)Tzn), 2-[3′-(triphenylen-2-yl)-1,1′-biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), and 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn). In particular, organic compounds having a phenanthroline skeleton, such as Bphen, BCP, NBphen, and mPPhen2P, are preferred, and an organic compound having a phenanthroline dimeric structure, such as mPPhen2P, is further preferred because of its excellent stability.

The second layer 117, which is a charge generation layer, is preferably formed with a composite material of a material having an acceptor property and an organic compound having a hole-transport property. As the organic compound having a hole-transport property used in the composite material, any of a variety of organic compounds such as aromatic amine compounds, heteroaromatic compounds, aromatic hydrocarbons, and high molecular compounds (e.g., oligomers, dendrimers, and polymers) can be used. Note that the organic compound having a hole-transport property preferably has a hole mobility of 1 × 10⁻⁶ cm²/Vs or higher. The material having a hole-transport property used in the composite material is preferably a compound having a condensed aromatic hydrocarbon ring or a π-electron rich heteroaromatic ring. As the condensed aromatic hydrocarbon ring, an anthracene ring, a naphthalene ring, or the like is preferable. As the π-electron rich heteroaromatic ring, a condensed aromatic ring having at least one of a pyrrole skeleton, a furan skeleton, and a thiophene skeleton is preferable; specifically, a carbazole ring, a dibenzothiophene ring, or a ring in which an aromatic ring or a heteroaromatic ring is further condensed to the carbazole ring or the dibenzothiophene ring is preferable.

Such an organic compound having a hole-transport property further preferably has any of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to the nitrogen of the amine through an arylene group may be used. Note that the organic compound having a hole-transport property preferably has an N,N-bis(4-biphenyl)amino group to enable fabricating a light-emitting device having a long lifetime.

Specific examples of the hole-transport material include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-y1)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-y1)-9,9′-spirobi-9H-fluoren-1-amine.

Examples of the aromatic amine compounds that can be used as the material having a hole-transport property include N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N-(3-methylphenyl)-N-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B).

As the substance having an acceptor property included in the second layer 117, it is possible to use an organic compound having an electron-withdrawing group (e.g., a halogen group or a cyano group); for example, 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylene-1,3,4,5,6,8,9,10-octafluoro-7H-pyren-2-ylidene)malononitrile can be used. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a very high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as molybdenum oxide, vanadium oxide, ruthenium oxide, tungsten oxide, or manganese oxide can be used, other than the above-described organic compounds.

The third layer 118 includes a substance having an electron-transport property and has a function of preventing an interaction between the first layer 119 and the second layer 117 and transferring electrons smoothly. The LUMO level of the substance having an electron-transport property included in the third layer 118 is preferably between the LUMO level of the acceptor substance in the second layer 117 and the LUMO level of the organic compound included in a layer (the first electron-transport layer 114_1 in the first light-emitting unit 501 in FIG. 1B) which is included in the light-emitting unit on the first electrode 101 side and is in contact with the intermediate layer 116. As a specific value of the energy level, the LUMO level of the substance having an electron-transport property in the third layer 118 is preferably higher than or equal to -5.0 eV, further preferably higher than or equal to -5.0 eV and lower than or equal to -3.0 eV. Note that as the substance having an electron-transport property in the third layer 118, a phthalocyanine-based material or a metal complex having a metal-oxygen bond and an aromatic ligand is preferably used.

Then, components of the above light-emitting device 130, other than the intermediate layer 116, are described.

### <<Structure of First Electrode>>

The first electrode 101 is the electrode including an anode. The first electrode 101 may have a stacked structure in which the layer in contact with the organic compound layer 103 functions as the anode. The anode is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide containing silicon or silicon oxide, indium oxide-zinc oxide, and indium oxide containing tungsten oxide and zinc oxide (IWZO). Such conductive metal oxide films are usually formed by a sputtering method, but may be formed by application of a sol-gel method or the like. In an example of the formation method, a film of indium oxide-zinc oxide is formed by a sputtering method using a target obtained by adding 1 wt% to 20 wt% of zinc oxide to indium oxide. Furthermore, a film of indium oxide containing tungsten oxide and zinc oxide (IWZO) can be formed by a sputtering method using a target in which 0.5 wt% to 5 wt% tungsten oxide and 0.1 wt% to 1 wt% zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), nitride of a metal material (e.g., titanium nitride), or the like can be used for the anode. Graphene can also be used for the anode. Note that an electrode material can be selected regardless of the work function when the composite material forming the second layer 117 in the above intermediate layer 116 is used for the layer (typically the hole-injection layer) in contact with the anode.

### <<Structure of Hole-Injection Layer>>

The hole-injection layer 111 is provided in contact with the anode and has a function of facilitating injection of holes into the organic compound layer 103 (the first light-emitting unit 501). The hole-injection layer 111 can be formed using a phthalocyanine-based compound such as phthalocyanine (abbreviation: H₂Pc) and copper phthalocyanine (abbreviation: CuPc), an aromatic amine compound such as 4,4′-bis[N-(4-diphenylaminophenyl)-N-phenylamino]biphenyl (abbreviation: DPAB) and 4,4′-bis(N-{4-[N-(3-methylphenyl)-N′-phenylamino]phenyl}-N-phenylamino)biphenyl (abbreviation: DNTPD), or a high molecular compound such as poly(3,4-ethylenedioxythiophene)/poly(styrenesulfonic acid) (abbreviation: PEDOT/PSS).

The hole-injection layer 111 may be formed with a substance having an electron-accepting property. As the substance having an acceptor property, any of the substances described as the acceptor substance used for the composite material forming the second layer 117 in the above intermediate layer 116 can be used similarly.

The composite material forming the second layer 117 in the above intermediate layer 116 may be similarly used to form the hole-injection layer 111.

In the hole-injection layer 111, it is further preferable that the organic compound having a hole-transport property used in the composite material have a relatively deep HOMO level higher than or equal to -5.7 eV and lower than or equal to -5.4 eV. Using the organic compound having a hole-transport property which has a relatively deep HOMO level in the composite material makes it easy to inject holes into the hole-transport layer and to obtain a light-emitting device having a long lifetime. In addition, when the organic compound having a hole-transport property used in the composite material has a relatively deep HOMO level, induction of holes can be inhibited properly so that the light-emitting device can have a longer lifetime.

The formation of the hole-injection layer 111 can improve the hole-injection property, which allows the light-emitting device to be driven at a low voltage.

Among substances having an acceptor property, the organic compound having an acceptor property is easy to use because it is easily deposited by vapor deposition.

The second light-emitting unit 502 includes no hole-injection layer because the second layer 117 in the intermediate layer 116 functions as a hole-injection layer; however, the second light-emitting unit 502 may include a hole-injection layer.

The hole-transport layer such as the first hole-transport layer 112_1 or the second hole-transport layer 112_2 includes an organic compound having a hole-transport property. The organic compound having a hole-transport property preferably has a hole mobility higher than or equal to 1 × 10⁻⁶ cm²/Vs.

Examples of the aforementioned organic compound that having a hole-transport property include compounds having an aromatic amine skeleton, such as 4,4′-bis[N-(1-naphthyl)-N-phenylamino]biphenyl (abbreviation: NPB), N,N-diphenyl-N,N-bis(3-methylphenyl)-4,4′-diaminobiphenyl (abbreviation: TPD), N,N′-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP); 9,9′-bis(biphenyl-4-yl)-3,3′-bi-9H-carbazole (abbreviation: BisBPCz), 9,9′-bis(1,1′-biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-9H,9′H-3,3′-bicarbazole (abbreviation: βNCCP), 9-(3-biphenyl)-9′(2-naphthyl)-X3,3′-bi-9H-carbazole (abbreviation: βNCCmBP), 9-(4-binaphthyl)-9′-(2-naphthyl)-x3,3′-bi-9H-carbazole (abbreviation: βNCCBP), 9,9′-di-2-naphthyl-3,3′-9H,9′H-bicarbazole (abbreviation: βNCCBP), 9-(2-naphthyl)-9′-[1,1′:4′,1″-terphenyl]-3-yl-3,3′-9H,9′H-bicarbazole, 9-(2-naphthyl)-9′-[1,1′:3′,1″-terphenyl]-3-yl-3,3′-9H,9′H-bicarbazole, 9-(2-naphthyl)-9′-[1,1′:3′,1″-terphenyl]-5′-yl-3,3′-9H,9′H-bicarbazole, 9-(2-naphthyl)-9′-[1,1′:4′,1″-terphenyl]-4-yl-3,3′-9H,9′H-bicarbazole, 9-(2-naphthyl)-9′-[1,1′:3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, and 9-(triphenylen-2-yl)-9′-[1,1′:3′,1″-terphenyl]-4-yl-3,3′-9H,9′H-bicarbazole; compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in drive voltage. Note that any of the substances given as examples of the organic compound having a hole-transport property that is used for the composite material in the hole-injection layer 111 can also be suitably used as the material included in the hole-transport layer.

### <<Structure of Light-Emitting Layer>>

The light-emitting layer such as the light-emitting layer 113, the first light-emitting layer 113_1, or the second light-emitting layer 113_2 preferably includes a light-emitting substance and a host material. The light-emitting layer may additionally include other materials. Alternatively, the light-emitting layer may be a stack of two layers with different compositions.

As the light-emitting substance, fluorescent substances, phosphorescent substances, substances exhibiting thermally activated delayed fluorescence (TADF), or other light-emitting substances may be used.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N-bis[4-(9-phenyl-9H-fluoren-9-yl)phenyl]pyrene-1,6-diamine (abbreviation: 1,6FLPAPm), N,N-bis(3-methylphenyl)-N,N-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis(N,N,N-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N,N,N′,N′,N″,N″-octaphenyldibenzo[g,p]chrysene-2,7,10,15-tetraamine (abbreviation: DBC1), coumarin 30, N-(9,10-diphenyl-2-anthryl)-N,1,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), 9,10-bis(2-biphenyl)-2-(N,N′,N′-triphenyl-1,4-phenylenediamin-N-yl)anthracene (abbreviation: 2DPABPhA), 9,10-bis(1,1′-biphenyl-2-yl)-N-[4-(9H-carbazol-9-yl)phenyl]-N- phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl] ethenyl }-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N-diphenyl-N,N′-(1,6-pyrene-diyl)bis[(6-phenylbenzo[b]naphtho[1,2-d]furan)-8-amine] (abbreviation: 1,6BnfAPm-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPm, 1,6mMemFLPAPrn, and 1,6BnfAPm-03 are particularly preferable because of their high hole-trapping properties and high emission efficiency or high reliability.

Examples of the material that can be used when a phosphorescent substance is used as the light-emitting substance in the light-emitting layer are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[1-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIracac). These compounds emit blue phosphorescent light and have an emission peak in the wavelength range from 450 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]); [2-d3-methyl-8-(2-pyridinyl-κN)benzofuro[2,3-b]pyridine-κC]bis[2-(5-d3-methyl-2-pyridinyl-κN²)phenyl-κC]iridium(III) (abbreviation: Ir(5mppy-d3)₂(mbfpypy-d3)), {[2-(methyl-d3)-8-[4-(1-methylethyl-1-d)-2-pyridinyl-κN]benzofuro[2,3-b]pyridin-7-yl-κC}bis{5-(methyl-d3)-2-[5-(methyl-d3)-2-pyridinyl-κN]phenyl-κC}iridium(III) (abbreviation: Ir(5mtpy-d6)₂(mbfpypy-iPr-d4)), [2-d3-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy-d3)]), and [2-(4-methyl-5-phenyl-2-pyridinyl-κN)phenyl-κC]bis[2-(2-pyridinyl-κN)phenyl-κC]iridium(III) (abbreviation: [Ir(ppy)₂(mdppy)]); and a rare earth metal complex such as tris(acetylacetonato) (monophenanthroline)terbium(III) (abbreviation: [Tb(acac)₃(Phen)]). These are mainly compounds that emit green phosphorescent light and have an emission peak in the wavelength range from 500 nm to 600 nm. Note that organometallic iridium complexes including a pyrimidine skeleton have distinctively high reliability or emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]) and bis(1-phenylisoquinolinato-N,C^{2′})iridium(III) acetylacetonate (abbreviation: [Ir(piq)₂(acac)]); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescent light and have an emission peak in the wavelength range from 600 nm to 700 nm. Furthermore, the organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structure formulae.

### [Chemical Formula 12]

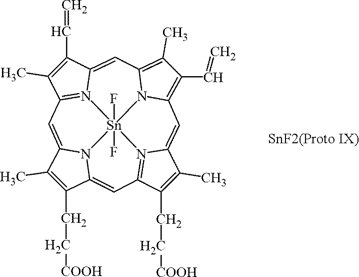

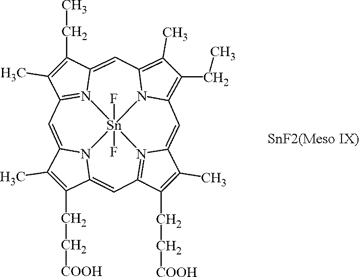

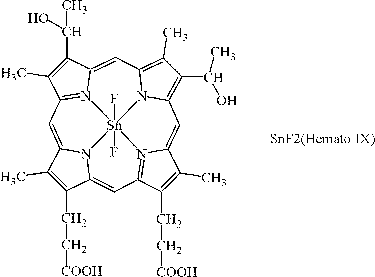

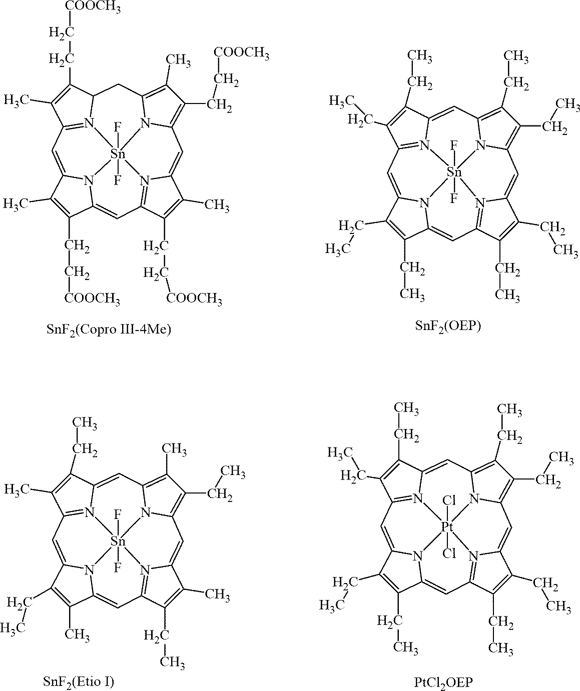

Alternatively, a heterocyclic compound having one or both of a π-electron rich heteroaromatic ring and a π-electron deficient heteroaromatic ring that is represented by the following structure formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. Such a heterocyclic compound is preferable because of having excellent electron-transport and hole-transport properties owing to a π-electron rich heteroaromatic ring and a π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferred because of their high stability and reliability. In particular, a benzofuropyrimidine skeleton, a benzothienopyrimidine skeleton, a benzofuropyrazine skeleton, and a benzothienopyrazine skeleton are preferred because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferred because the electron-donating property of the π-electron rich heteroaromatic ring and the electron-accepting property of the π-electron deficient heteroaromatic ring are both improved, the energy difference between the S1 level and the T1 level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

### [Chemical Formula 13]

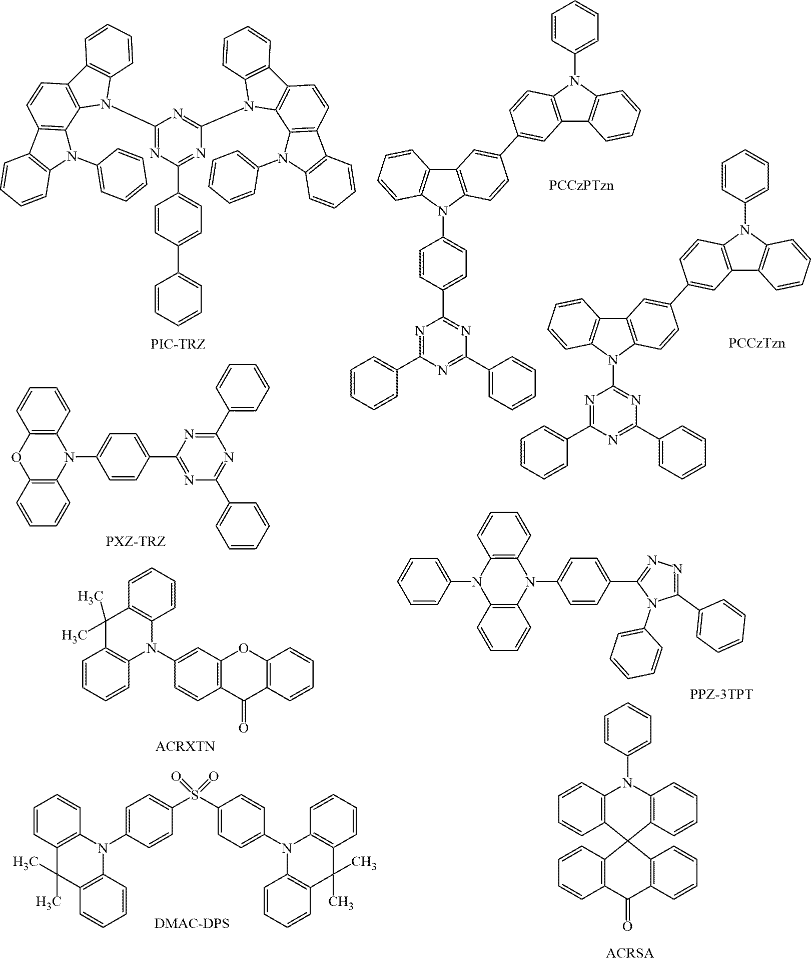

Alternatively, a TADF material whose singlet excited state and triplet excited state are in a thermal equilibrium state may be used. Since such a TADF material enables a short emission lifetime (excitation lifetime), an efficiency decrease of a light-emitting device in a high-luminance region can be inhibited. Specifically, a material having the following molecular structure can be used.

### [Chemical Formula 14]

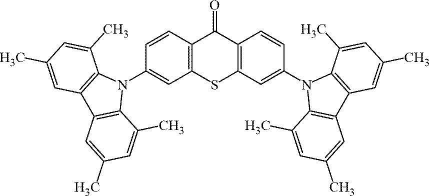

Note that a TADF material is a material having a small difference between the S1 level and the T1 level and a function of converting triplet excitation energy into singlet excitation energy by reverse intersystem crossing. Thus, a TADF material enables upconversion of triplet excitation energy into singlet excitation energy (i.e., reverse intersystem crossing) using a small amount of thermal energy and efficiently generate a singlet excited state. In addition, the triplet excitation energy can be converted into luminescence.

An exciplex whose excited state is formed of two kinds of substances has an extremely small difference between the S1 level and the T1 level and functions as a TADF material capable of converting triplet excitation energy into singlet excitation energy.

A phosphorescent spectrum observed at a low temperature (e.g., 77 K to 10 K) is used for an index of the T1 level. When the level of energy with a wavelength of the line obtained by extrapolating a tangent to the fluorescent spectrum at a tail on the short wavelength side is the S1 level and the level of energy with a wavelength of the line obtained by extrapolating a tangent to the phosphorescent spectrum at a tail on the short wavelength side is the T1 level, the difference between the S1 level and the T1 level of the TADF material is preferably smaller than or equal to 0.3 eV, further preferably smaller than or equal to 0.2 eV.

When a TADF material is used as the light-emitting substance, the S1 level of the host material is preferably higher than that of the TADF material. In addition, the T1 level of the host material is preferably higher than that of the TADF material.

As the host material in the light-emitting layer, various carrier-transport materials such as materials having an electron-transport property and/or materials having a hole-transport property, and the TADF materials can be used.

As the material having a hole-transport property, the aforementioned material given as the material having a hole-transport property can be favorably used similarly.

As the material having an electron-transport property, the aforementioned material given as the material having electron-transport property can be favorably used similarly.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S1 level of the TADF material is preferably higher than that of the fluorescent substance in order that high emission efficiency can be achieved. Furthermore, the T1 level of the TADF material is preferably higher than the S1 level of the fluorescent substance. Therefore, the T1 level of the TADF material is preferably higher than that of the fluorescent substance.

It is also preferable to use a TADF material that emits light whose wavelength overlaps with the wavelength on a lowest-energy-side absorption band of the fluorescent substance, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transportation or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring. Examples of the condensed aromatic ring or the condensed heteroaromatic ring include a phenanthrene skeleton, a stilbene skeleton, an acridone skeleton, a phenoxazine skeleton, and a phenothiazine skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferred because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further condensed to carbazole because the HOMO level thereof is shallower than that of carbazole by approximately 0.1 eV and thus holes enter the host material easily. In particular, the host material preferably has a dibenzocarbazole skeleton because the HOMO level thereof is shallower than that of carbazole by approximately 0.1 eV so that holes enter the host material easily, the hole-transport property is improved, and the heat resistance is increased. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4-(9-phenyl-9H-fluoren-9-yl)biphenyl-4′-yl]anthracene (abbreviation: FLPPA), 9-(1-naphthyl)-10-[4-(2-naphthyl)phenyl]anthracene (abbreviation: αN-βNPAnth), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)-benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthracenyl]phenyl}-2-ethyl-1H-benzimidazole (abbreviation: EtBImPBPhA). In particular, CzPA, cgDBCzPA, 2mBnfPPA, and PCzPA have excellent characteristics and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength on a lowest-energy-side absorption band of the light-emitting substance, in which case energy can be transferred smoothly and light emission can be obtained efficiently. The use of such a structure is preferable because the drive voltage can also be reduced.

Note that at least one of the materials forming an exciplex may be a phosphorescent substance. In this case, triplet excitation energy can be efficiently converted into singlet excitation energy by reverse intersystem crossing.

Combination of a material having an electron-transport property and a material having a hole-transport property whose HOMO level is higher than or equal to that of the material having an electron-transport property is preferable for forming an exciplex efficiently. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength than the emission spectra of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient PL lifetime of the mixed film has longer lifetime components or has a larger proportion of delayed components than that of each of the materials, observed by comparison of transient photoluminescence (PL) of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

### <<Structure of Electron-Transport Layer>>

The electron-transport layer such as electron-transport layer 114, the first electron-transport layer 114_1, or the second electron-transport layer 114_2 includes a substance having an electron-transport property. The material having an electron-transport property is preferably a substance with an electron mobility higher than or equal to 1 × 10⁻⁷ cm²/Vs, preferably higher than or equal to 1 × 10⁻⁶ cm²/Vs, when the square root of electric field strength [V/cm] is 600. Note that any other substance can be used as long as the substance has an electron-transport property higher than a hole-transport property. An organic compound including a π-electron deficient heteroaromatic ring is preferable as the above organic compound. The organic compound including a π-electron deficient heteroaromatic ring is preferably one or more of an organic compound including a heteroaromatic ring having a polyazole skeleton, an organic compound including a heteroaromatic ring having a pyridine skeleton, an organic compound including a heteroaromatic ring having a diazine skeleton, and an organic compound including a heteroaromatic ring having a triazine skeleton.

As the organic compound having an electron-transport property which can be used for the above electron-transport layer, the organic compound that can be used as the organic compound having an electron-transport property in the first layer of the above intermediate layer 116 can be used similarly. Among the above organic compounds, the organic compound having a heteroaromatic ring having a diazine skeleton, the organic compound having a heteroaromatic ring having a pyridine skeleton, and the organic compound having a heteroaromatic ring having a triazine skeleton have high reliability and thus are preferable. In particular, the organic compound that includes a heteroaromatic ring having a diazine (pyrimidine or pyrazine) skeleton and the organic compound that includes a heteroaromatic ring having a triazine skeleton have a good electron-transport property to contribute to a reduction in driving voltage.

The electron mobility of the electron-transport layer in the case where the square root of the electric field strength [V/cm] is 600 is preferably higher than or equal to 1 × 10⁻⁷ cm²/Vs and lower than or equal to 5 × 10⁻⁵ cm²/Vs. The amount of electrons injected into the light-emitting layer can be controlled by the reduction in the electron-transport property of the electron-transport layer, whereby the light-emitting layer can be prevented from having excess electrons. It is particularly preferable to employ this structure when the hole-injection layer is formed using a composite material that includes a material having a hole-transport property with a relatively deep HOMO level of -5.7 eV or higher and -5.4 eV or lower, in which case a long lifetime can be achieved. In this case, the material having an electron-transport property preferably has a HOMO level of -6.0 eV or higher.

### <<Structure of Electron-Injection Layer>>

The electron-injection layer 115 can be formed using an alkali metal, a rare earth metal, or an alkaline earth metal, a compound thereof, or a complex thereof such as lithium fluoride (LiF), cesium fluoride (CsF), calcium fluoride (CaF₂), 8-quinolinolato lithium (abbreviation: Liq), or ytterbium (Yb). An electride or a layer that is formed using a substance having an electron-transport property and that includes an alkali metal, an alkaline earth metal, or a compound thereof can be used as the electron-injection layer 115. Examples of the electride include a substance in which electrons are added at high concentration to calcium oxide-aluminum oxide.

Note that as the electron-injection layer 115, it is possible to use a layer containing a substance that has an electron-transport property (preferably an organic compound having a bipyridine skeleton) and contains a fluoride of the alkali metal or the alkaline earth metal at a concentration higher than that at which the electron-injection layer 115 becomes in a microcrystalline state (50 wt% or higher). Since the layer has a low refractive index, a light-emitting device including the layer can have high external quantum efficiency.

Any of the organic compounds of one embodiment of the present invention described in Embodiment 1 can be used for the electron-injection layer 115. The electron-injection layer 115 may include a substance having an electron-transport property in addition to any of the organic compounds of one embodiment of the present invention described in Embodiment 1.

### <<Structure of Second Electrode>>

The second electrode 102 is the electrode including a cathode. The second electrode 102 may have a stacked structure in which the layer in contact with the organic compound layer 103 functions as the cathode. For the cathode, a metal, an alloy, an electrically conductive compound, or a mixture thereof each having a low work function (specifically, lower than or equal to 3.8 eV) or the like can be used. Specific examples of such a cathode material are elements belonging to Group 1 or 2 of the periodic table, such as alkali metals (e.g., lithium (Li) or cesium (Cs)), magnesium (Mg), calcium (Ca), and strontium (Sr), alloys containing these elements (e.g., MgAg and AlLi), rare earth metals such as europium (Eu) and ytterbium (Yb), and alloys containing these rare earth metals. However, when the electron-injection layer is provided between the second electrode 102 and the electron-transport layer, a variety of conductive materials such as Al, Ag, ITO, or indium oxide-tin oxide containing silicon or silicon oxide can be used for the cathode regardless of the work function.

When the second electrode 102 is formed using a material that transmits visible light, the light-emitting device can emit light from the second electrode 102 side.

Films of these conductive materials can be deposited by a dry process such as a vacuum evaporation method or a sputtering method, an ink-jet method, a spin coating method, or the like. Alternatively, a wet process using a sol-gel method or a wet process using a paste of a metal material may be employed.

Furthermore, any of a variety of methods can be used for forming the organic compound layer 103, regardless of a dry method or a wet method. For example, a vacuum evaporation method, a gravure printing method, an offset printing method, a screen printing method, an ink-jet method, a spin coating method, or the like may be used.

Different methods may be used to form the electrodes or the layers described above.

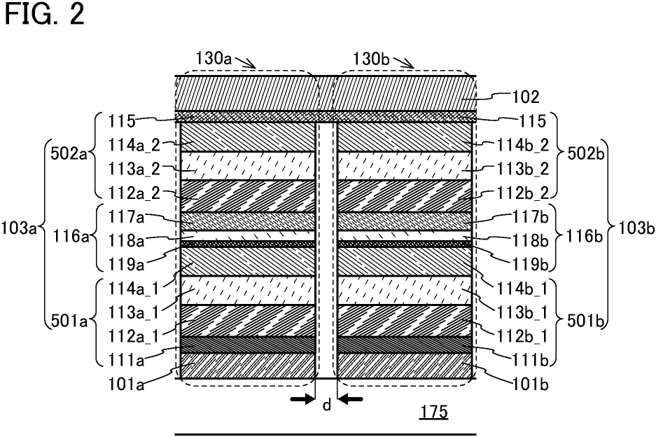

FIG. 2 illustrates two light-emitting devices (a light-emitting device 130a and a light-emitting device 130b) which are adjacent to each other in a light-emitting apparatus of one embodiment of the present invention.

The light-emitting device 130a includes an organic compound layer 103a between a first electrode 101a and the second electrode 102 over an insulating layer 175. In the organic compound layer 103a, a first light-emitting unit 501a and a second light-emitting unit 502a are stacked with an intermediate layer 116a interposed therebetween. Although two light-emitting units are stacked in the example shown in FIG. 2, three or more light-emitting units may be stacked. The first light-emitting unit 501a includes a hole-injection layer 111a, a first hole-transport layer 112a_1, a first light-emitting layer 113a_1, and a first electron-transport layer 114a_1. The intermediate layer 116a includes a second layer 117a, a third layer 118a, and a first layer 119a. The third layer 118a may be present or absent. The second light-emitting unit 502a includes a second hole-transport layer 112a_2, a second light-emitting layer 113a_2, a second electron-transport layer 114a_2, and the electron-injection layer 115.

The light-emitting device 130b includes an organic compound layer 103b between a first electrode 101b and the second electrode 102 over the insulating layer 175. In the organic compound layer 103b, a first light-emitting unit 501b and a second light-emitting unit 502b are stacked with an intermediate layer 116b interposed therebetween. Although two light-emitting units are stacked in the example shown in FIG. 2, three or more light-emitting units may be stacked. The first light-emitting unit 501b includes a hole-injection layer 111b, a first hole-transport layer 112b_1, a first light-emitting layer 113b_1, and a first electron-transport layer 114b_1. The intermediate layer 116b includes a second layer 117b, a third layer 118b, and a first layer 119b. The third layer 118b may be present or absent. The second light-emitting unit 502b includes a second hole-transport layer 112b_2, a second light-emitting layer 113b_2, a second electron-transport layer 114b_2, and the electron-injection layer 115.

The electron-injection layer 115 and the second electrode 102 are preferably a continuous layer shared between the light-emitting devices 130a and 130b. Except for the electron-injection layer 115, the organic compound layers 103a and 103b are isolated from each other because they are processed by a photolithography method after the formation of the layer to be the second electron-transport layer 114a_2 and after the formation of the layer to be the second electron-transport layer 114b_2. The end portions (outlines) of the layers in the organic compound layer 103a except the electron-injection layer 115 are substantially aligned in the direction perpendicular to the substrate due to the processing by a photolithography method. The end portions (outlines) of the layers in the organic compound layer 103b except the electron-injection layer 115 are substantially aligned in the direction perpendicular to the substrate due to the processing by a photolithography method.

Since the organic compound layers are processed by a photolithography method, the distance d between the first electrodes 101a and 101b can be shorter than that in the case of employing mask vapor deposition; the distance d can be longer than or equal to 2 µm and shorter than or equal to 5 µm.

This embodiment can be combined as appropriate with the other embodiments or the examples. In this specification, in the case where a plurality of structure examples are shown in one embodiment, the structure examples can be combined as appropriate.

### Embodiment 3

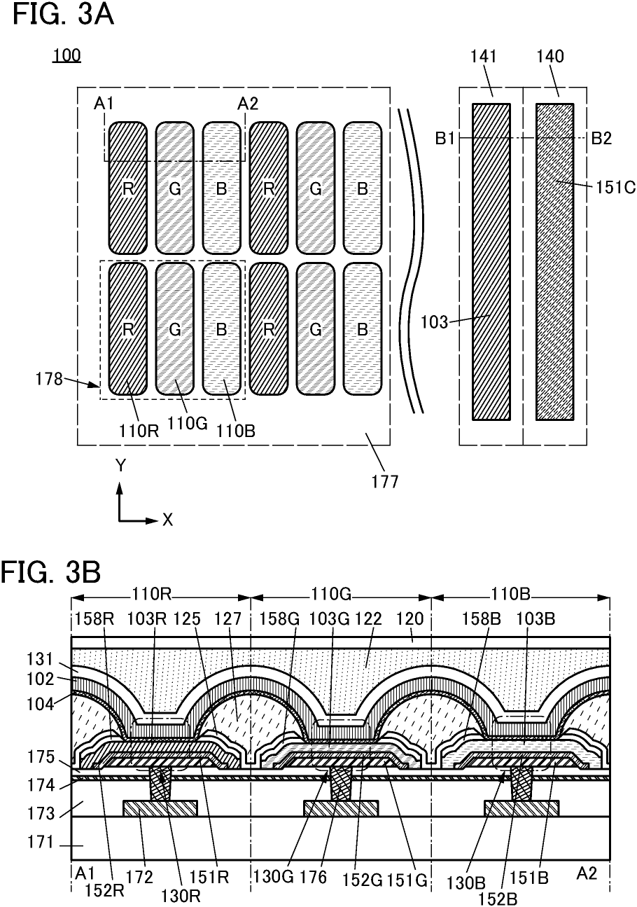

As illustrated in FIGS. 3A and 3B, a plurality of the light-emitting devices 130 are formed over the insulating layer 175 to constitute a display apparatus. In this embodiment, the display apparatus of one embodiment of the present invention will be described in detail.

A display apparatus 100 includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110.” As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and Y, and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

A connection portion 140 and a region 141 are provided outside the pixel portion 177, and the region 141 is positioned between the pixel portion 177 and the connection portion 140. The organic compound layer 103 is provided in the region 141. A conductive layer 151C is provided in the connection portion 140.

Although FIGS. 3A and 3B illustrate an example where the region 141 and the connection portion 140 are positioned on the right side of the pixel portion 177, the positions of the region 141 and the connection portion 140 are not particularly limited. The number of regions 141 and the number of connection portions 140 can each be one or more.

FIG. 3B is a cross-sectional view along the dashed-dotted line A1-A2 in FIG. 3A. As illustrated in FIG. 3B, the display apparatus 100 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is bonded to the protective layer 131 with a resin layer 122. An inorganic insulating layer 125 and an insulating layer 127 over the inorganic insulating layer 125 are preferably provided between the adjacent light-emitting devices 130.

Although each of the inorganic insulating layer 125 and the insulating layer 127 looks like a plurality of layers in the cross-sectional view in FIG. 3B, each of the inorganic insulating layer 125 and the insulating layer 127 is preferably one continuous layer when the display apparatus 100 is seen from above. In other words, the insulating layer 127 preferably includes opening portions over the first electrode.

In FIG. 3B, a light-emitting device 130R, a light-emitting device 130G, and a light-emitting device 130B are shown as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, 130G, or 130B may emit visible light of another color or infrared light.

The display apparatus of one embodiment of the present invention can be, for example, a top-emission display apparatus where light is emitted in the direction opposite to a substrate over which light-emitting devices are formed. Note that the display apparatus of one embodiment of the present invention may be of a bottom emission type.

Examples of a light-emitting substance included in the light-emitting device 130 include organic compounds or organometallic complexes such as a substance emitting fluorescent light (a fluorescent material), a substance emitting phosphorescent light (a phosphorescent material), and a substance exhibiting thermally activated delayed fluorescence (a thermally activated delayed fluorescent (TADF) material). Other examples include inorganic compounds (e.g., a quantum dot material).

The light-emitting device 130R has a structure as described in Embodiment 2. The light-emitting device 130R includes a first electrode (pixel electrode) including a conductive layer 151R and a conductive layer 152R, an organic compound layer 103R over the first electrode, a common layer 104 over the organic compound layer 103R, and a second electrode (common electrode) 102 over the common layer 104. Although the common layer 104 is not necessarily provided, it is preferable to provide the common layer 104 to reduce damage to the organic compound layer 103R during processing. In the case where the common layer 104 is provided, the common layer 104 is preferably an electron-injection layer. Furthermore, in the case where the common layer 104 is provided, a stack of the organic compound layer 103R and the common layer 104 corresponds to the organic compound layer 103 described in Embodiment 2.

The light-emitting device 130G has a structure as described in Embodiment 2. The light-emitting device 130G includes the first electrode (pixel electrode) including a conductive layer 151G and a conductive layer 152G, an organic compound layer 103G over the first electrode, the common layer 104 over the organic compound layer 103G, and the second electrode (common electrode) 102 over the common layer. Although the common layer 104 is not necessarily provided, it is preferable to provide the common layer 104 to reduce damage to the organic compound layer 103G during processing. In the case where the common layer 104 is provided, the common layer 104 is preferably an electron-injection layer. Furthermore, in the case where the common layer 104 is provided, a stack of the organic compound layer 103G and the common layer 104 corresponds to the organic compound layer 103 described in Embodiment 2.

The light-emitting device 130B has a structure as described in Embodiment 2. The light-emitting device 130B includes the first electrode (pixel electrode) including a conductive layer 151B and a conductive layer 152B, an organic compound layer 103B over the first electrode, the common layer 104 over the organic compound layer 103B, and the second electrode (common electrode) 102 over the common layer. Although the common layer 104 is not necessarily provided, it is preferable to provide the common layer 104 to reduce damage to the organic compound layer 103B during processing. In the case where the common layer 104 is provided, the common layer 104 is preferably an electron-injection layer. Furthermore, in the case where the common layer 104 is provided, a stack of the organic compound layer 103B and the common layer 104 corresponds to the organic compound layer 103 described in Embodiment 2.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, the organic compound layers 103G, and the organic compound layers 103B are island-shaped layers that are independent of each other; alternatively, an organic compound layer of the light-emitting devices of one emission color may be independent of an organic compound layer of the light-emitting devices of another emission color. Providing the island-shaped organic compound layer 103 in each of the light-emitting devices 130 can suppress leakage current between the adjacent light-emitting devices 130 even in a high-resolution display apparatus. This can prevent crosstalk, so that a display apparatus with extremely high contrast can be obtained. Specifically, a display apparatus having high current efficiency at low luminance can be obtained.

The island-shaped organic compound layer 103 is formed by forming an EL film and processing the EL film by a photolithography method.

The organic compound layer 103 is preferably provided to cover the top surface and the side surface of the first electrode (pixel electrode) of the light-emitting device 130. In this case, the aperture ratio of the display apparatus 100 can be easily increased as compared to the structure where an end portion of the organic compound layer 103 is positioned inward from an end portion of the pixel electrode. Covering the side surface of the pixel electrode of the light-emitting device 130 with the organic compound layer 103 can inhibit the pixel electrode from being in contact with the second electrode 102; hence, a short circuit of the light-emitting device 130 can be inhibited. Furthermore, the distance between the light-emitting region (i.e., the region overlapping with the pixel electrode) in the organic compound layer 103 and the end portion of the organic compound layer 103 can be increased. Since the end portion of the organic compound layer 103 might be damaged by processing, using a region that is away from the end portion of the organic compound layer 103 as the light-emitting region can increase the reliability of the light-emitting device 130.

In the display apparatus of one embodiment of the present invention, the first electrode (pixel electrode) of the light-emitting device preferably has a stacked-layer structure. For example, in the example illustrated in FIG. 3B, the first electrode of the light-emitting device 130 is a stack of the conductive layer 151 and the conductive layer 152. In the case where the display apparatus 100 is of a top emission type and the pixel electrode of the light-emitting device 130 functions as an anode, for example, the conductive layer 151 preferably has high visible light reflectance, and the conductive layer 152 preferably has a visible-light-transmitting property and a work function higher than that of the conductive layer 151. In the case where the display apparatus 100 is of a top emission type, the higher the visible light reflectance of the pixel electrode is, the higher the efficiency of extraction of the light emitted by the organic compound layer 103 is. In the case where the pixel electrode functions as an anode, the higher the work function of the pixel electrode is, the easier it is to inject holes into the organic compound layer 103. Accordingly, when the pixel electrode of the light-emitting device 130 is a stack of the conductive layer 151 with high visible light reflectance and the conductive layer 152 with a high work function, the light-emitting device 130 can have high light extraction efficiency and a low drive voltage.

In the case where the conductive layer 151 has high visible light reflectance, the visible light reflectance of the conductive layer 151 is higher than or equal to 40% and lower than or equal to 100%, preferably higher than or equal to 70% and lower than or equal to 100%, for example. When used as a electrode having a property of transmitting visible light, the conductive layer 152 preferably has a visible light transmittance higher than or equal to 40%, for example.

Here, such a pixel electrode being a stack composed of a plurality of layers might change in quality as a result of, for example, a reaction occurring between the plurality of layers. For example, when a film formed after the formation of the pixel electrode is removed by a wet etching method, contact of a chemical solution with the pixel electrode might cause galvanic corrosion.

In view of the above, the conductive layer 152 is formed to cover the top surface and the side surface of the conductive layer 151 in the display apparatus 100 of this embodiment. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display apparatus 100 to be manufactured by a high-yield method and to be accordingly inexpensive. In addition, generation of a defect in the display apparatus 100 can be inhibited, which makes the display apparatus 100 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a work function of higher than or equal to 4.0 eV, for example.

The conductive layer 151 and the conductive layer 152 may each be a stack of a plurality of layers containing different materials. In this case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

The conductive layer 151 preferably has a side surface with a tapered shape. Specifically, the end portion of the conductive layer 151 preferably has a tapered shape with a taper angle of less than 90°. In that case, the conductive layer 152 provided along the end portion of the conductive layer 151 also has a tapered shape. When the side surface of the conductive layer 152 has a tapered shape, coverage with the organic compound layer 103 provided along the side surface of the conductive layer 152 can be improved.

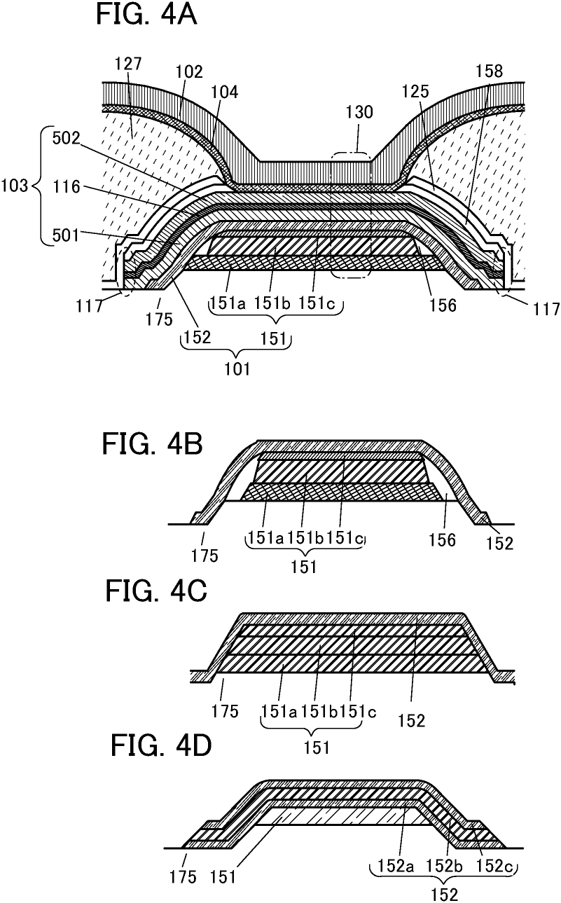

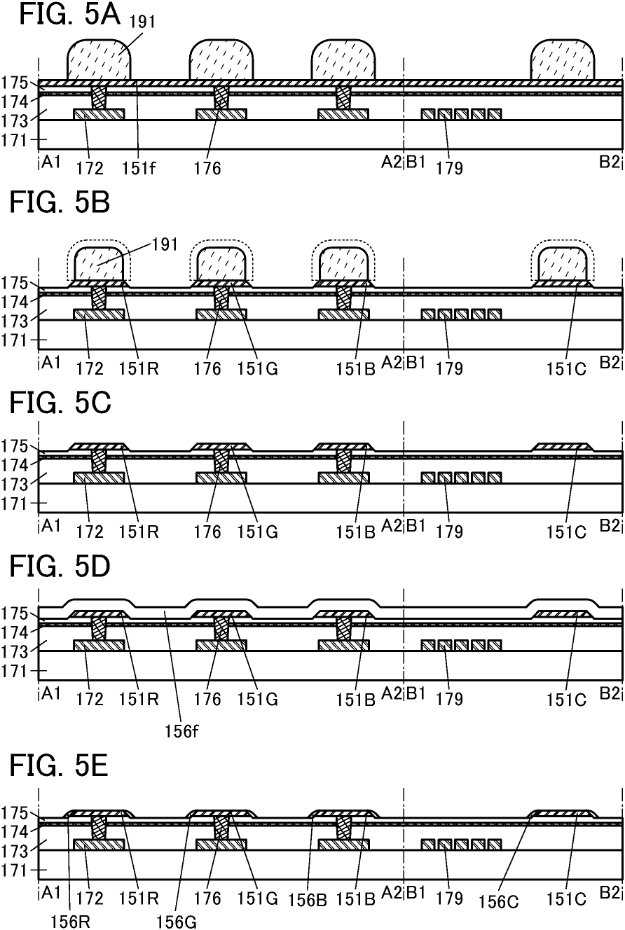

FIG. 4A illustrates the cases where the conductive layer 151 has a stacked-layer structure of a plurality of layers containing different materials. As illustrated in FIG. 4A, the conductive layer 151 includes a conductive layer 151a, a conductive layer 151b over the conductive layer 151a, and a conductive layer 151c over the conductive layer 151b. In other words, the conductive layer 151 illustrated in FIG. 4A has a three-layer structure. In the case where the conductive layer 151 is a stack of a plurality of layers as described above, the visible light reflectance of at least one of the layers included in the conductive layer 151 is made higher than that of the conductive layer 152.

In the examples illustrated in FIG. 4A, the conductive layer 151b is interposed between the conductive layers 151a and 151c. A material that is less likely to change in quality than the conductive layer 151b is preferably used for the conductive layers 151a and 151c. The conductive layer 151a can be formed using, for example, a material that is less likely to migrate owing to contact with the insulating layer 175 than the material for the conductive layer 151b. The conductive layer 151c can be formed using a material an oxide of which has lower electrical resistivity than an oxide of the material used for the conductive layer 151b and which is less likely to be oxidized than the conductive layer 151b.

In this manner, the structure in which the conductive layer 151b is interposed between the conductive layers 151a and 151c can expand the range of choices for the material for the conductive layer 151b. The conductive layer 151b, for example, can thus have higher visible light reflectance than at least one of the conductive layers 151a and 151c. For example, aluminum can be used for the conductive layer 151b. The conductive layer 151b may be formed using an alloy containing aluminum. The conductive layer 151a can be formed using titanium; titanium has lower visible light reflectance than aluminum but is less likely to migrate by contact with the insulating layer 175 than aluminum. Furthermore, the conductive layer 151c can be formed using titanium; titanium is less likely to be oxidized than aluminum and an oxide of titanium has lower electrical resistivity than aluminum oxide, although titanium has lower visible light reflectance than aluminum.

The conductive layer 151c may be formed using silver or an alloy containing silver. Silver is characterized by its visible light reflectance higher than that of titanium. In addition, silver is characterized by being less likely to be oxidized than aluminum, and silver oxide is characterized by its electrical resistivity lower than that of aluminum oxide. Thus, the conductive layer 151c formed using silver or an alloy containing silver can suitably increase the visible light reflectance of the conductive layer 151 and inhibit an increase in the electric resistance of the pixel electrode due to oxidation of the conductive layer 151b. Here, as the alloy containing silver, an alloy of silver, palladium, and copper (also referred to as Ag-Pd-Cu or APC) can be used, for example. When the conductive layer 151c is formed using silver or an alloy containing silver and the conductive layer 151b is formed using aluminum, the visible light reflectance of the conductive layer 151c can be higher than that of the conductive layer 151b. Here, the conductive layer 151b may be formed using silver or an alloy containing silver. The conductive layer 151a may be formed using silver or an alloy containing silver.

Meanwhile, a film formed using titanium has better processability in etching than a film formed using silver. Thus, use of titanium for the conductive layer 151c can facilitate formation of the conductive layer 151c. Note that a film formed using aluminum also has better processability in etching than a film formed using silver.

The conductive layer 151 having a stacked-layer structure of a plurality of layers as described above can improve the characteristics of the display apparatus. For example, the display apparatus 100 can have high light extraction efficiency and high reliability.

Here, in the case where the light-emitting device 130 has a microcavity structure, use of silver or an alloy containing silver, i.e., a material with high visible light reflectance, for the conductive layer 151c can favorably increase the light extraction efficiency of the display apparatus 100.

As already described above, the conductive layer 151 preferably has a side surface with a tapered shape. Specifically, the side surface of the conductive layer 151 preferably has a tapered shape with a taper angle of less than 90°. For example, in the conductive layer 151 illustrated in FIG. 4A, the side surface of at least one of the conductive layer 151a, the conductive layer 151b, and the conductive layer 151c preferably has a tapered shape.

The conductive layer 151 shown in FIG. 4A can be formed by a photolithography method. Specifically, first, a conductive film to be the conductive layer 151a, a conductive film to be the conductive layer 151b, and a conductive film to be the conductive layer 151c are sequentially formed. Next, a resist mask is formed over the conductive film to be the conductive layer 151c. Then, the conductive films in the region not overlapped by the resist mask are removed by etching. Here, when the conductive films are processed under conditions where the resist mask is easily recessed (reduced in size) as compared to the case where the conductive layer 151 is formed such that the side surface does not have a tapered shape (i.e., the conductive layer 151 is formed to have a perpendicular side surface), the side surface of the conductive layer 151 can have a tapered shape.

Here, when the conductive films are processed under conditions where the resist mask is easily recessed (reduced in size), the conductive films might be easily processed in the horizontal direction. That is, the etching sometimes might become more isotropic than in the case where the conductive layer 151 is formed to have a perpendicular side surface.

In the case where the conductive layer 151 is a stack of a plurality of layers composed of different materials, there is sometimes a difference in how easy the plurality of layers are processed in the horizontal direction. For example, the conductive layer 151a, the conductive layer 151b, and the conductive layer 151c are sometimes different in readiness to be processed in the horizontal direction.

In that case, after the processing of the conductive film, as illustrated in FIG. 4A, the side surface of the conductive layer 151b is sometimes positioned inward from the side surfaces of the conductive layers 151a and 151c and results in the formation of a protruding portion. This might impair coverage of the conductive layer 151 with the conductive layer 152 to cause step disconnection of the conductive layer 152.

In view of this, an insulating layer 156 is preferably provided as illustrated in FIG. 4A. FIG. 4A shows an example in which the insulating layer 156 is provided over the conductive layer 151a to include a region overlapping with the side surface of the conductive layer 151b. In this structure, occurrence of step disconnection or thinning of the conductive layer 152 due to the protruding portion can be inhibited, so that poor connection or an increase in drive voltage can be inhibited.

Although FIG. 4A illustrates the structure in which the side surface of the conductive layer 151b is entirely covered with the insulating layer 156, part of the side surface of the conductive layer 151b is not necessarily covered with the insulating layer 156. Also in a pixel electrode with a later-described structure, part of the side surface of the conductive layer 151b is not necessarily covered with the insulating layer 156.

In the case where the conductive layer 151 has the structure illustrated in FIG. 4A, the conductive layer 152 is provided to cover the conductive layers 151a, 151b, and 151c and the insulating layer 156 and to be electrically connected to the conductive layers 151a, 151b, and 151c. This can prevent a chemical solution from coming into contact with the conductive layers 151a, 151b, and 151c when a film formed after formation of the conductive layer 152 is removed by a wet etching method, for example. It is thus possible to inhibit occurrence of corrosion in the conductive layers 151a, 151b, and 151c. Hence, the display apparatus 100 can be manufactured by a high-yield method. Moreover, the display apparatus 100 can have high reliability since generation of defects is inhibited therein.

Here, the insulating layer 156 preferably has a curved surface as illustrated in FIG. 4A. In this case, a step-cut in the conductive layer 152 covering the insulating layer 156 is less likely to occur than in the case where the insulating layer 156 has a perpendicular side surface (a side surface parallel to the Z direction), for example. In addition, a step-cut in the conductive layer 152 covering the insulating layer 156 is less likely to occur also in the case where the side surface of the insulating layer 156 has a tapered shape, or specifically, a tapered shape with a taper angle of less than 90°, than in the case where the insulating layer 156 has a perpendicular side surface, for example. As described above, the display apparatus 100 can be manufactured by a high-yield method. Moreover, the display apparatus 100 can have high reliability since generation of defects is inhibited therein.

FIG. 4A illustrates a structure in which the side surface of the conductive layer 151b is positioned inward from that of the conductive layer 151a and that of the conductive layer 151c; however, one embodiment of the present invention is not limited thereto. For example, the side surface of the conductive layer 151b may be positioned outward from that of the conductive layer 151a. The side surface of the conductive layer 151b may be positioned outward from that of the conductive layer 151c.

FIGS. 4B to 4D illustrate other structures of the first electrode 101. FIG. 4B illustrates a variation structure of the first electrode 101 in FIG. 4A, in which the insulating layer 156 covers the side surfaces of the conductive layers 151a, 151b, and 151c instead of covering only the side surface of the conductive layer 151b.

FIG. 4C illustrates a variation structure of the first electrode 101 in FIG. 4A, in which the insulating layer 156 is not provided.

FIG. 4D illustrates a variation structure of the first electrode 101 in FIG. 4A, in which the conductive layer 151 does not have a stacked-layer structure but the conductive layer 152 has a stacked-layer structure.

A conductive layer 152a has higher adhesion to a conductive layer 152b than the insulating layer 175 does, for example. For the conductive layer 152a, an oxide containing one or more selected from indium, tin, zinc, gallium, titanium, aluminum, and silicon, for example, can be used. For example, it is preferable to use a conductive oxide containing one or more of indium oxide, indium tin oxide, indium zinc oxide, zinc oxide, zinc oxide containing gallium, titanium oxide, indium titanium oxide, zinc titanate, aluminum zinc oxide, indium zinc oxide containing gallium, indium zinc oxide containing aluminum, indium tin oxide containing silicon, indium zinc oxide containing silicon, and the like. Accordingly, peeling of the conductive layer 152b can be inhibited. The conductive layer 152b is not in contact with the insulating layer 175.

The conductive layer 152b is a layer whose visible light reflectance (e.g., reflectance with respect to light with a predetermined wavelength in a range greater than or equal to 400 nm and less than 750 nm) is higher than that of the conductive layers 151, 152a, and 152c. The visible light reflectance of the conductive layer 152b can be, for example, higher than or equal to 70% and lower than or equal to 100%, and is preferably higher than or equal to 80% and lower than or equal to 100%, further preferably higher than or equal to 90% and lower than or equal to 100%. For the conductive layer 152b, a material having higher visible light reflectance than aluminum can be used, for example. Specifically, for the conductive layer 152b, silver or an alloy containing silver can be used, for example. An example of the alloy containing silver is an alloy of silver, palladium, and copper (APC). In the above manner, the display apparatus 100 can have high light extraction efficiency. Note that a metal other than silver may be used for the conductive layer 152b.

When the conductive layers 151 and 152 serve as the anode, a layer having a high work function is preferably used as the conductive layer 152c. The conductive layer 152c has a higher work function than the conductive layer 152b, for example. For the conductive layer 152c, a material similar to the material usable for the conductive layer 152a can be used, for example. For example, the conductive layers 152a and 152c can be formed using the same kind of material. For example, in the case where indium tin oxide is used for the conductive layer 152a, indium tin oxide can also be used for the conductive layer 152c.

When the conductive layers 151 and 152 serve as the cathode, a layer having a low work function is preferably used. The conductive layer 152c has a lower work function than the conductive layer 152b, for example.

The conductive layer 152c is preferably a layer having high visible light transmittance (e.g., transmittance with respect to light with a predetermined wavelength in a range greater than or equal to 400 nm and less than 750 nm). For example, the visible light transmittance of the conductive layer 152c is preferably higher than that of the conductive layers 151 and 152b. The visible light transmittance of the conductive layer 152c can be, for example, higher than or equal to 60% and lower than or equal to 100%, and is preferably higher than or equal to 70% and lower than or equal to 100%, further preferably higher than or equal to 80% and lower than or equal to 100%. Accordingly, the amount of light absorbed by the conductive layer 152c among light emitted from the organic compound layer 103 can be reduced. As described above, the conductive layer 152b under the conductive layer 152c can be a layer having high visible light reflectance. Thus, the display apparatus 100 can have high light extraction efficiency.

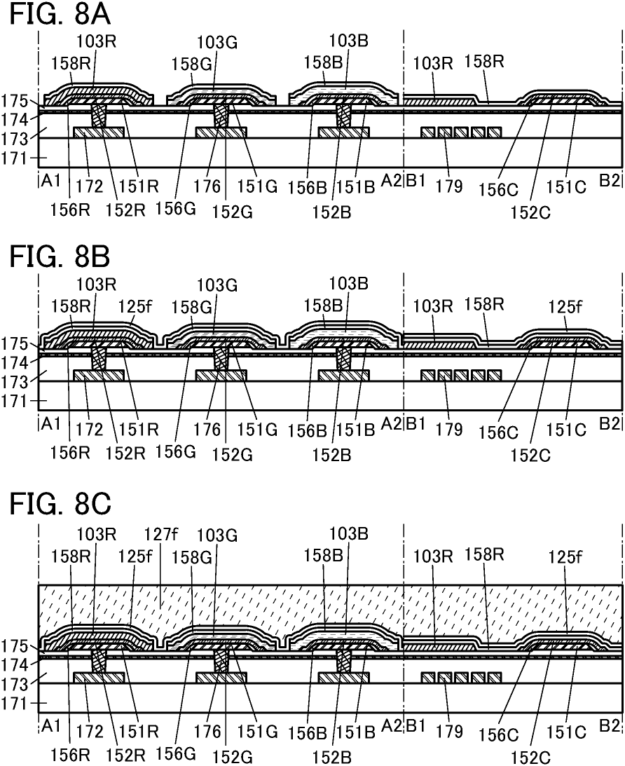

Next, an exemplary method for manufacturing the display apparatus 100 having the structure illustrated in FIG. 3A is described with reference to FIGS. 8A to 8C, FIGS. 9A to 9C, FIGS. 10A to 10C, FIGS. 11A and 11B, FIGS. 12A and 12B, and FIGS. 13A to 13D.

### [Manufacturing Method Example]

Thin films included in the display apparatus (e.g., insulating films, semiconductor films, and conductive films) can be formed by a sputtering method, a chemical vapor deposition (CVD) method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic CVD (MOCVD) method.

Thin films included in the display apparatus (e.g., insulating films, semiconductor films, and conductive films) can also be formed by a wet process such as spin coating, dipping, spray coating, ink-jetting, dispensing, screen printing, offset printing, doctor blade coating, slit coating, roll coating, curtain coating, or knife coating.

Specifically, for fabrication of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the display apparatus can be processed by a photolithography method, for example. Alternatively, a nanoimprinting method, a sandblasting method, a lift-off method, or the like may be used to process thin films. Alternatively, island-shaped thin films may be directly formed by a film formation method using a shielding mask such as a metal mask.

There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

As light used for exposure in the photolithography method, for example, light with an i-line (wavelength: 365 nm), light with a g-line (wavelength: 436 nm), light with an h-line (wavelength: 405 nm), or light in which the i-line, the g-line, and the h-line are mixed can be used. Alternatively, ultraviolet rays, KrF laser light, ArF laser light, or the like can be used. Exposure may be performed by liquid immersion exposure technique. As the light for exposure, extreme ultraviolet (EUV) light or X-rays may also be used. Furthermore, instead of the light used for exposure, an electron beam can be used. It is preferable to use EUV light, X-rays, or an electron beam to perform extremely minute processing. Note that when exposure is performed by scanning of a beam such as an electron beam, a photomask is not needed.

For etching of thin films, a dry etching method, a wet etching method, a sandblast method, or the like can be used.

First, as illustrated in FIG. 5A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate that has heat resistance high enough to withstand at least heat treatment performed later can be used. When an insulating substrate is used, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like; a compound semiconductor substrate of silicon germanium or the like; or an SOI substrate.

Next, as illustrated in FIG. 5A, openings reaching the conductive layer 172 are formed in the insulating layers 175, 174, and 173. Then, the plugs 176 are formed to fill the openings.

Next, as illustrated in FIG. 5A, a conductive film 151f to be the conductive layers 151R, 151G, 151B, and 151C is formed over the plugs 176 and the insulating layer 175. The conductive film 151f can be formed by a sputtering method or a vacuum evaporation method, for example. A metal material can be used for the conductive film 151f, for example.

Subsequently, a resist mask 191 is formed over the conductive film 151f as illustrated in FIG. 5A. The resist mask 191 can be formed by application of a photosensitive material (photoresist), light exposure, and development.

Subsequently, as illustrated in FIG. 5B, the conductive film 151f in a region that is not overlapped by the resist mask 191, for example, is removed by an etching method, specifically, a dry etching method, for instance. Note that in the case where the conductive film 151f includes a layer formed using a conductive oxide such as indium tin oxide, for example, the layer may be removed by a wet etching method. In this manner, the conductive layer 151 is formed. In the case where part of the conductive film 151f is removed by a dry etching method, for example, a recessed portion may be formed in a region of the insulating layer 175 that is not overlapped by the conductive layer 151.

Next, the resist mask 191 is removed as illustrated in FIG. 5C. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Then, as illustrated in FIG. 5D, an insulating film 156f to be an insulating layer 156R, an insulating layer 156G, an insulating layer 156B, and an insulating layer 156C is formed over the conductive layer 151R, the conductive layer 151G, the conductive layer 151B, the conductive layer 151C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film containing silicon, a nitride insulating film containing silicon, an oxynitride insulating film containing silicon, a nitride oxide insulating film containing silicon, or the like can be used as the insulating film 156f. For the insulating film 156f, silicon oxynitride can be used, for example.

Subsequently, as illustrated in FIG. 5E, the insulating film 156f is processed to form the insulating layers 156R, 156G, 156B, and 156C. The insulating layer 156 can be formed by performing etching substantially uniformly on the top surface of the insulating film 156f, for example. Such uniform etching for planarization is also referred to as etch back treatment. Note that the insulating layer 156 may be formed by a photolithography method.

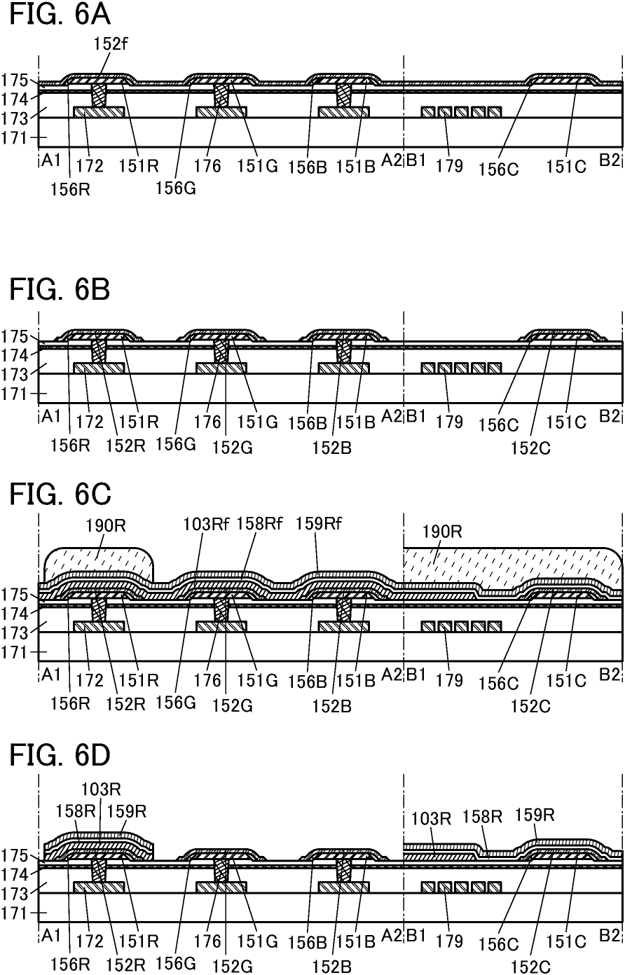

Then, as illustrated in FIG. 6A, a conductive film 152f to be the conductive layers 152R, 152G, and 152B and a conductive layer 152C is formed over the conductive layers 151R, 151G, 151B, and 151C and the insulating layers 156R, 156G, 156B, 156C, and 175. Specifically, the conductive film 152f is formed to cover the conductive layers 151R, 151G, 151B, and 151C and the insulating layers 156R, 156G, 156B, and 156C, for example.

The conductive film 152f can be formed by a sputtering method or a vacuum evaporation method, for example. A conductive oxide can be used for the conductive film 152f, for example. The conductive film 152f can be a stack of a film formed using a metal material and a film formed thereover using a conductive oxide. For example, the conductive film 152f can be a stack of a film formed using titanium, silver, or an alloy containing silver and a film formed thereover using a conductive oxide.

The conductive film 152f can be formed by an ALD method. In this case, for the conductive film 152f, an oxide containing one or more selected from indium, tin, zinc, gallium, titanium, aluminum, and silicon can be used. In this case, the conductive film 152f can be formed by repeating a cycle of introduction of a precursor (generally referred to as a metal precursor or the like in some cases), purge of the precursor, introduction of an oxidizer (generally referred to as a reactant, a non-metal precursor, or the like in some cases), and purge of the oxidizer. Here, in the case where an oxide film containing a plurality of kinds of metals (e.g., an indium tin oxide film) is formed as the conductive film 152f, the composition of the metals can be controlled by varying the number of cycles for different kinds of precursors.

For example, in the case where an indium tin oxide film is formed as the conductive film 152f, after a precursor containing indium is introduced, the precursor is purged, and an oxidizer is introduced to form an In-O film, and then a precursor containing tin is introduced, the precursor is purged, and an oxidizer is introduced to form a Sn-O film. Here, when the number of cycles of forming an In-O film is larger than the number of cycles of forming a Sn-O film, the number of In atoms contained in the conductive film 152f can be larger than the number of Sn atoms contained therein.

For example, to form a zinc oxide film as the conductive film 152f, a Zn-O film is formed in the above procedure. As another example, to form an aluminum zinc oxide film as the conductive film 152f, a Zn-O film and an Al-O film are formed in the above procedure. As another example, to form a titanium oxide film as the conductive film 152f, a Ti-O film is formed in the above procedure. As another example, to form an indium tin oxide film containing silicon as the conductive film 152f, an In-O film, a Sn-O film, and a Si-O film are formed in the above procedure. As another example, to form a zinc oxide film containing gallium, a Ga-O film and a Zn-O film are formed in the above procedure.

As a precursor containing indium, it is possible to use, for example, triethylindium, trimethylindium, or [1,1,1-trimethyl-N-(trimethylsilyl)amide]-indium. As a precursor containing tin, it is possible to use, for example, tin chloride or tetrakis(dimethylamido)tin. As a precursor containing zinc, it is possible to use, for example, diethylzinc or dimethylzinc. As a precursor containing gallium, it is possible to use, for example, triethylgallium. As a precursor containing titanium, it is possible to use, for example, titanium chloride, tetrakis(dimethylamido)titanium, or tetraisopropyl titanate. As a precursor containing aluminum, it is possible to use, for example, aluminum chloride or trimethylaluminum. As a precursor containing silicon, it is possible to use, for example, trisilylamine, bis(diethylamino)silane, tris(dimethylamino)silane, bis(tert-butylamino)silane, or bis(ethylmethylamino)silane. As the oxidizer, water vapor, oxygen plasma, or an ozone gas can be used.

Then, as illustrated in FIG. 6B, the conductive film 152f is processed by a photolithography method, for example, whereby the conductive layers 152R, 152G, 152B, and 152C are formed. Specifically, after a resist mask is formed, part of the conductive film 152f is removed by an etching method, for example. The conductive film 152f can be removed by a wet etching method, for example. The conductive film 152f may be removed by a dry etching method. Through the above steps, the pixel electrode including the conductive layer 151 and the conductive layer 152 is formed.

Next, hydrophobization treatment is preferably performed on the conductive layer 152. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and an organic compound layer 103 formed in a later step and suppress film peeling. Note that the hydrophobization treatment is not necessarily performed.

Next, as illustrated in FIG. 6C, an organic compound film 103Rf to be an organic compound layer 103R is formed over the conductive layers 152R, 152G, and 152B and the insulating layer 175.

As illustrated in FIG. 6C, the organic compound film 103Rf is not formed over the conductive layer 152C. For example, a mask for specifying a film formation area (also referred to as an area mask, a rough metal mask, or the like to distinguish from a fine metal mask) is used, so that the organic compound film 103Rf can be formed only in a desired region. Employing a film formation step using an area mask and a processing step using a resist mask enables a light-emitting device to be manufactured by a relatively easy process.

The organic compound film 103Rf can be formed by an evaporation method, specifically a vacuum evaporation method, for example. The organic compound film 103Rf may be formed by a transfer method, a printing method, an ink-jet method, a coating method, or the like.

Next, as illustrated in FIG. 6C, a sacrificial film 158Rf to be a sacrificial layer 158R and a mask film 159Rf to be a mask layer 159R are sequentially formed over the organic compound film 103Rf, the conductive layer 152C, and the insulating layer 175.

Although this embodiment shows an example where a mask film having a two-layer structure of the sacrificial film 158Rf and the mask film 159Rf is formed, a mask film may have a single-layer structure or a stacked-layer structure of three or more layers.

Providing the sacrificial layer over the organic compound film 103Rf can reduce damage to the organic compound film 103Rf in the manufacturing process of the display apparatus, resulting in an increase in reliability of the light-emitting device.

As the sacrificial film 158Rf, a film that is highly resistant to the process conditions for the organic compound film 103Rf, specifically, a film having high etching selectivity with respect to the organic compound film 103Rf is used. For the mask film 159Rf, a film having high etching selectivity with respect to the sacrificial film 158Rf is used.

The sacrificial film 158Rf and the mask film 159Rf are formed at a temperature lower than the upper temperature limit of the organic compound film 103Rf. The typical substrate temperatures in formation of the sacrificial film 158Rf and the mask film 159Rf are each lower than or equal to 200° C., preferably lower than or equal to 150° C., further preferably lower than or equal to 120° C., still further preferably lower than or equal to 100° C., yet still further preferably lower than or equal to 80° C.

The sacrificial film 158Rf and the mask film 159Rf are preferably films that can be removed by a wet etching method. The use of a wet etching method can reduce damage to the organic compound film 103Rf in processing of the sacrificial film 158Rf and the mask film 159Rf, as compared to the case of using a dry etching method.

The sacrificial film 158Rf and the mask film 159Rf can be formed by a sputtering method, an ALD method (including a thermal ALD method or a PEALD method), a CVD method, or a vacuum evaporation method, for example. Alternatively, the sacrificial film 158Rf and the mask film 159Rf may be formed by the above-described wet process.

Note that the sacrificial film 158Rf that is formed over and in contact with the organic compound film 103Rf is preferably formed by a formation method that is less likely to damage the organic compound film 103Rf than a formation method of the mask film 159Rf. For example, the sacrificial film 158Rf is preferably formed by an ALD method or a vacuum evaporation method rather than a sputtering method.

As each of the sacrificial film 158Rf and the mask film 159Rf, one or more of a metal film, an alloy film, a metal oxide film, a semiconductor film, an organic insulating film, and an inorganic insulating film, for example, can be used.

For each of the sacrificial film 158Rf and the mask film 159Rf, it is preferable to use a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials, for example. It is particularly preferable to use a low-melting-point material such as aluminum or silver. It is preferable to use a metal material that can block ultraviolet rays for one or both of the sacrificial film 158Rf and the mask film 159Rf, in which case the organic compound film 103Rf can be inhibited from being irradiated with ultraviolet rays and deterioration of the organic compound film 103Rf can be suppressed.

The sacrificial film 158Rf and the mask film 159Rf can each be formed using a metal oxide such as In—Ga—Zn oxide, indium oxide, In—Zn oxide, In—Sn oxide, indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or indium tin oxide containing silicon.

In addition, in place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used. In particular, Mis preferably one or more of gallium, aluminum, and yttrium.

As each of the sacrificial film and the mask film, a film containing a material having a light-blocking property, particularly with respect to ultraviolet rays, is preferably used. Although a variety of materials such as a metal, an insulator, a semiconductor, and a metalloid that have a property of blocking ultraviolet rays can be used as a light-blocking material, each of the sacrificial film and the mask film is preferably a film capable of being processed by etching and is particularly preferably a film having good processability because part or the whole of each of the sacrificial film and the mask film is removed in a later step.

For example, a semiconductor material with excellent compatibility with a semiconductor manufacturing process, such as silicon or germanium, is preferably used for the sacrificial film and the mask film. An oxide or a nitride of the semiconductor material can be used. A non-metallic material such as carbon or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

When a film containing a material having a property of blocking ultraviolet rays is used as each of the sacrificial film and the mask film, the organic compound layer can be inhibited from being irradiated with ultraviolet rays in a light exposure step, for example. The organic compound layer is inhibited from being damaged by ultraviolet rays, so that the reliability of the light-emitting device can be improved.

Note that the same effect is obtained when a film containing a material having a property of blocking ultraviolet rays is used for an after-mentioned inorganic insulating film 125f.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film. For example, an inorganic insulating material such as aluminum oxide, hafnium oxide, or silicon oxide can be used for the sacrificial film 158Rf and the mask film 159Rf. As the sacrificial film 158Rf and the mask film 159Rf, aluminum oxide films can be formed by an ALD method, for example. An ALD method is preferably used, in which case damage to a base (in particular, the organic compound layer) can be reduced.

For example, an inorganic insulating film (e.g., an aluminum oxide film) formed by an ALD method can be used as the sacrificial film 158Rf, and an inorganic film (e.g., an In—Ga—Zn oxide film, an aluminum film, or a tungsten film) formed by a sputtering method can be used as the mask film 159Rf.

Note that the same inorganic insulating film can be used for both the sacrificial film 158Rf and an inorganic insulating layer 125 that is to be formed later. For example, an aluminum oxide film formed by an ALD method can be used for both the sacrificial film 158Rf and the inorganic insulating layer 125. For the sacrificial film 158Rf and the inorganic insulating layer 125, the same deposition conditions may be used or different deposition conditions may be used. For example, when the sacrificial film 158Rf is formed under conditions similar to those of the inorganic insulating layer 125, the sacrificial film 158Rf can be an insulating layer having a high barrier property against at least one of water and oxygen. Meanwhile, since the sacrificial film 158Rf is a layer a large part or the whole of which is to be removed in a later step, it is preferable that the processing of the sacrificial film 158Rf be easy. Therefore, the sacrificial film 158Rf is preferably formed with a substrate temperature lower than that for formation of the inorganic insulating layer 125.

One or both of the sacrificial film 158Rf and the mask film 159Rf may be formed using an organic material. For example, as the organic material, a material that can be dissolved in a solvent chemically stable with respect to at least the uppermost film of the organic compound film 103Rf may be used. Specifically, a material that will be dissolved in water or an alcohol can be suitably used. In forming a film of such a material, it is preferable to apply the material dissolved in a solvent such as water or an alcohol by a wet process and then perform heat treatment for evaporating the solvent. At this time, the heat treatment is preferably performed in a reduced-pressure atmosphere, in which case the solvent can be removed at a low temperature in a short time and thermal damage to the organic compound film 103Rf can be reduced accordingly.

The sacrificial film 158Rf and the mask film 159Rf may be formed using an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluorine resin like perfluoropolymer.

For example, an organic film (e.g., a PVA film) formed by an evaporation method or any of the above wet processes can be used as the sacrificial film 158Rf, and an inorganic film (e.g., a silicon nitride film) formed by a sputtering method can be used as the mask film 159Rf.

Subsequently, a resist mask 190R is formed over the mask film 159Rf as illustrated in FIG. 6C. The resist mask 190R can be formed by application of a photosensitive material (photoresist), light exposure, and development.

The resist mask 190R may be formed using either a positive resist material or a negative resist material.

The resist mask 190R is provided at a position overlapping the conductive layer 152R. The resist mask 190R is preferably provided also at a position overlapping the conductive layer 152C. This can inhibit the conductive layer 152C from being damaged during the process of manufacturing the display apparatus. Note that the resist mask 190R is not necessarily provided over the conductive layer 152C. The resist mask 190R is preferably provided to cover the area from the end portion of the organic compound film 103Rf to the end portion of the conductive layer 152C (the end portion closer to the organic compound film 103Rf), as illustrated in the cross-sectional view along the line B1-B2 in FIG. 6C.

Next, as illustrated in FIG. 6D, part of the mask film 159Rf is removed using the resist mask 190R, whereby the mask layer 159R is formed. The mask layer 159R remains over the conductive layers 152R and 152C. After that, the resist mask 190R is removed. Then, part of the sacrificial film 158Rf is removed using the mask layer 159R as a mask (also referred to as a hard mask), whereby the sacrificial layer 158R is formed.

Each of the sacrificial film 158Rf and the mask film 159Rf can be processed by a wet etching method or a dry etching method. The sacrificial film 158Rf and the mask film 159Rf are preferably processed by isotropic etching.

The use of a wet etching method can reduce damage to the organic compound film 103Rf in processing of the sacrificial film 158Rf and the mask film 159Rf, as compared to the case of using a dry etching method. In the case of using a wet etching method, it is preferable to use a chemical solution of a developer, an aqueous solution of tetramethylammonium hydroxide (TMAH), dilute hydrofluoric acid, oxalic acid, phosphoric acid, acetic acid, nitric acid, or a mixed solution thereof, for example.

Since the organic compound film 103Rf is not exposed in the processing of the mask film 159Rf, the range of choice for a processing method for the mask film 159Rf is wider than that for the sacrificial film 158Rf. Specifically, even in the case where a gas containing oxygen is used as the etching gas in the processing of the mask film 159Rf, deterioration of the organic compound film 103Rf can be suppressed.

In the case of using a dry etching method to process the sacrificial film 158Rf, deterioration of the organic compound film 103Rf can be suppressed by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

For example, in the case where an aluminum oxide film formed by an ALD method is used as the sacrificial film 158Rf, part of the sacrificial film 158Rf can be removed by a dry etching method using CHF₃ and He or a combination of CHF₃, He, and CH₄. In the case where an In—Ga—Zn oxide film formed by a sputtering method is used as the mask film 159Rf, part of the mask film 159Rf can be removed by a wet etching method using diluted phosphoric acid. Alternatively, part of the mask film 159Rf may be removed by a dry etching method using CH₄ and Ar. Alternatively, part of the mask film 159Rf can be removed by a wet etching method using diluted phosphoric acid. In the case where a tungsten film formed by a sputtering method is used as the mask film 159Rf, part of the mask film 159Rf can be removed by a dry etching method using a combination of SF₆, CF₄, and O₂ or a combination of CF₄, Cl₂, and O₂.

The resist mask 190R can be removed by a method similar to that for the resist mask 191. For example, the resist mask 190R can be removed by ashing using oxygen plasma. Alternatively, an oxygen gas and any of CF₄, C₄F₈, SF₆, CHF₃, Cl₂, H₂O, BCl₃, and a Group 18 element such as He may be used. Alternatively, the resist mask 190R may be removed by wet etching. At this time, the sacrificial film 158Rf is positioned on the outermost surface, and the organic compound film 103Rf is not exposed; thus, the organic compound film 103Rf can be inhibited from being damaged in the step of removing the resist mask 190R. In addition, the range of choice of the method for removing the resist mask 190R can be widened.

Next, as illustrated in FIG. 6D, the organic compound film 103Rf is processed, so that the organic compound layer 103R is formed. For example, part of the organic compound film 103Rf is removed using the mask layer 159R and the sacrificial layer 158R as a hard mask, whereby the organic compound layer 103R is formed.

Accordingly, as illustrated in FIG. 6D, the stacked-layer structure of the organic compound layer 103R, the sacrificial layer 158R, and the mask layer 159R remains over the conductive layer 152R. The conductive layers 152G and 152B are exposed.

In the example illustrated in FIG. 6D, the end portion of the organic compound layer 103R is positioned outward from the end portion of the conductive layer 152R. Such a structure can increase the aperture ratio of the pixel. Although not illustrated in FIG. 6D, by the above etching treatment, a recessed portion may be formed in the insulating layer 175 in a region not overlapped by the organic compound layer 103R.

Since the organic compound layer 103R covers the top surface and the side surface of the conductive layer 152R, the subsequent steps can be performed without exposure of the conductive layer 152R. If the end portion of the conductive layer 152R is exposed, there is a possibility that corrosion is caused in an etching step, for example. A product generated by corrosion of the conductive layer 152R may be unstable, and for example, might be dissolved in a solution when wet etching is performed and might be scattered in an atmosphere when dry etching is performed. By dissolution of the product in a solution or scattering of the product in the atmosphere, the product might be attached to a subject surface and the side surface of the organic compound layer 103R, for example, which might adversely affect the characteristics of the light-emitting device or form a leak path between a plurality of light-emitting devices. In a region where the end portion of the conductive layer 152R is exposed, adhesion between layers in contact with each other might be lowered, which might be likely to cause peeling of the organic compound layer 103R or the conductive layer 152R.

Accordingly, the structure where the organic compound layer 103R covers the top surface and the side surface of the conductive layer 152R can improve the yield and characteristics of the light-emitting device, for example.

As described above, the resist mask 190R is preferably provided to cover the area from the end portion of the organic compound layer 103R to the end portion of the conductive layer 152C (the end portion closer to the organic compound layer 103R) in the cross section B1-B2. Thus, as illustrated in FIG. 6D, the sacrificial layer 158R and the mask layer 159R are provided to cover the area from the end portion of the organic compound layer 103R to the end portion of the conductive layer 152C (the end portion closer to the organic compound layer 103R) in the cross section B1-B2. Hence, the insulating layer 175 can be inhibited from being exposed in the cross section B1-B2, for example. This can prevent the insulating layers 175, 174, and 173 from being partly removed by etching and thus prevent the conductive layer 179 from being exposed. Accordingly, the conductive layer 179 can be inhibited from being unintentionally electrically connected to another conductive layer. For example, a short circuit between the conductive layer 179 and a common electrode 155 formed in a later step can be suppressed.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be suppressed by not using a gas containing oxygen as the etching gas.

A gas containing oxygen may be used as the etching gas. When the etching gas contains oxygen, the etching rate can be increased. Therefore, the etching can be performed under a low-power condition while an adequately high etching rate is maintained. Accordingly, damage to the organic compound film 103Rf can be reduced. Furthermore, a defect such as attachment of a reaction product generated during the etching can be inhibited.

In the case of using a dry etching method, it is preferable to use a gas containing at least one of H₂, CF₄, C₄F₈, SF₆, CHF₃, Cl₂, H₂O, BCl₃, and a Group 18 element such as He and Ar as the etching gas, for example. Alternatively, a gas containing oxygen and at least one of the above is preferably used as the etching gas. Alternatively, an oxygen gas may be used as the etching gas. Specifically, for example, a gas containing H₂ and Ar or a gas containing CF₄ and He can be used as the etching gas. As another example, a gas containing CF₄, He, and oxygen can be used as the etching gas. As another example, a gas containing H₂ and Ar and a gas containing oxygen can be used as the etching gas.

As described above, in one embodiment of the present invention, the mask layer 159R is formed in the following manner: the resist mask 190R is formed over the mask film 159Rf and part of the mask film 159Rf is removed using the resist mask 190R. After that, part of the organic compound film 103Rf is removed using the mask layer 159R as a hard mask, so that the organic compound layer 103R is formed. In other words, the organic compound layer 103R is formed by processing the organic compound film 103Rf by a photolithography method. Note that part of the organic compound film 103Rf may be removed using the resist mask 190R. Then, the resist mask 190R may be removed.

Next, hydrophobization treatment for the conductive layer 152G, for example, is preferably performed. At the time of processing the organic compound film 103Rf, a surface of the conductive layer 152G changes to have hydrophilic properties in some cases, for example. The hydrophobization treatment for the conductive layer 152G, for example, can increase the adhesion between the conductive layer 152G and a layer to be formed in a later step (which is the organic compound layer 103G here) and suppress film peeling. Note that the hydrophobization treatment is not necessarily performed.

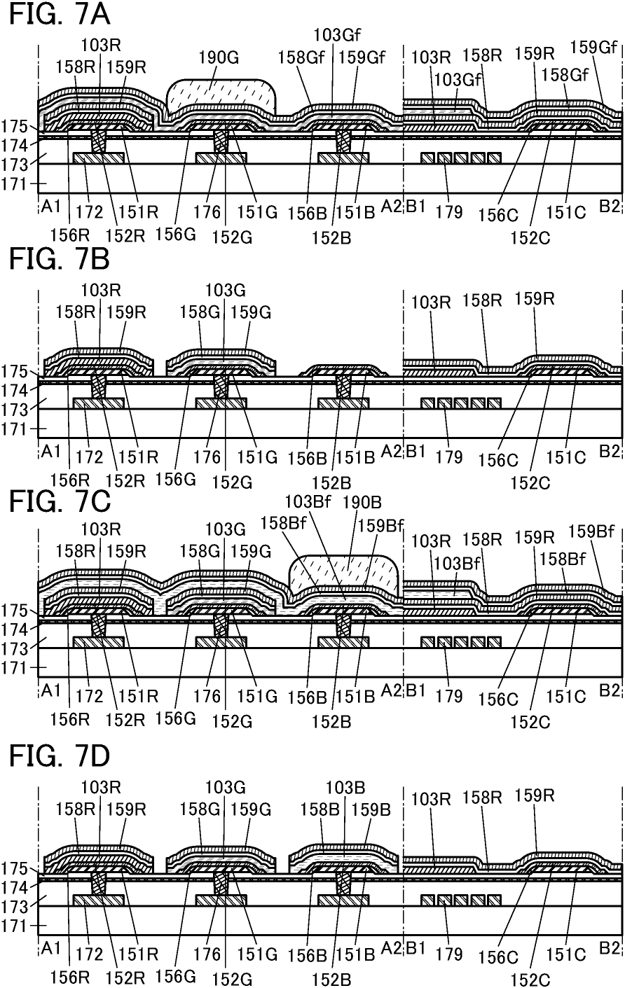

Next, as illustrated in FIG. 7A, an organic compound film 103Gf to be the organic compound layer 103G is formed over the conductive layer 152G, the conductive layer 152B, the mask layer 159R, and the insulating layer 175.

The organic compound film 103Gf can be formed by a method similar to that for forming the organic compound film 103Rf. The organic compound film 103Gf can have a structure similar to that of the organic compound film 103Rf.

Then, as illustrated in FIG. 7A, a sacrificial film 158Gf to be a sacrificial layer 158G and a mask film 159Gf to be a mask layer 159G are sequentially formed over the organic compound film 103Gf and the mask layer 159R. After that, a resist mask 190G is formed. The materials and the formation methods of the sacrificial film 158Gf and the mask film 159Gf are similar to those for the sacrificial film 158Rf and the mask film 159Rf. The material and the formation method of the resist mask 190G are similar to those for the resist mask 190R.

The resist mask 190G is provided at a position overlapping the conductive layer 152G.

Subsequently, as illustrated in FIG. 7B, part of the mask film 159Gf is removed using the resist mask 190G, whereby the mask layer 159G is formed. The mask layer 159G remains over the conductive layer 152G. After that, the resist mask 190G is removed. Then, part of the sacrificial film 158Gf is removed using the mask layer 159G as a mask, whereby the sacrificial layer 158G is formed. Next, the organic compound film 103Gf is processed to form the organic compound layer 103G. For example, part of the organic compound film 103Gf is removed using the mask layer 159G and the sacrificial layer 158G as a hard mask to form the organic compound layer 103G.

Accordingly, as illustrated in FIG. 7B, the stacked-layer structure of the organic compound layer 103G, the sacrificial layer 158G, and the mask layer 159G remains over the conductive layer 152G. The mask layer 159R and the conductive layer 152B are exposed.

Next, hydrophobization treatment for the conductive layer 152B, for example, is preferably performed. At the time of processing the organic compound film 103Gf, a surface of the conductive layer 152B changes to have hydrophilic properties in some cases, for example. The hydrophobization treatment for the conductive layer 152B, for example, can increase the adhesion between the conductive layer 152B and a layer to be formed in a later step (which is the organic compound layer 103B here) and suppress film peeling. Note that the hydrophobization treatment is not necessarily performed.

Next, as illustrated in FIG. 7C, an organic compound film 103Bf to be the organic compound layer 103B is formed over the conductive layer 152B, the mask layer 159R, the mask layer 159G, and the insulating layer 175.

The organic compound film 103Bf can be formed by a method similar to that for forming the organic compound film 103Rf. The organic compound film 103Bf can have a structure similar to that of the organic compound film 103Rf.

Then, as illustrated in FIG. 7C, a sacrificial film 158Bf to be a sacrificial layer 158B and a mask film 159Bf to be a mask layer 159B are sequentially formed over the organic compound film 103Bf and the mask layer 159R. After that, a resist mask 190B is formed. The materials and the formation methods of the sacrificial film 158Bf and the mask film 159Bf are similar to those for the sacrificial film 158Rf and the mask film 159Rf. The material and the formation method of the resist mask 190B are similar to those for the resist mask 190R.

The resist mask 190B is provided at a position overlapping the conductive layer 152B.

Subsequently, as illustrated in FIG. 7D, part of the mask film 159Bf is removed using the resist mask 190B, whereby the mask layer 159B is formed. The mask layer 159B remains over the conductive layer 152B. After that, the resist mask 190B is removed. Then, part of the sacrificial film 158Bf is removed using the mask layer 159B as a mask, whereby the sacrificial layer 158B is formed. Next, the organic compound film 103Bf is processed to form the organic compound layer 103B. For example, part of the organic compound film 103Bf is removed using the mask layer 159B and the sacrificial layer 158B as a hard mask to form the organic compound layer 103B.

Accordingly, as illustrated in FIG. 7D, the stacked-layer structure of the organic compound layer 103B, the sacrificial layer 158B, and the mask layer 159B remains over the conductive layer 152B. The mask layers 159R and 159G are exposed.

Note that the side surfaces of the organic compound layers 103R, 103G, and 103B are preferably perpendicular or substantially perpendicular to their formation surfaces. For example, the angle between the formation surfaces and these side surfaces is preferably greater than or equal to 60° and less than or equal to 90°.

The distance between two adjacent layers among the organic compound layers 103R, 103G, and 103B, which are formed by a photolithography method as described above, can be reduced to less than or equal to 8 µm, less than or equal to 5 µm, less than or equal to 3 µm, less than or equal to 2 µm, or less than or equal to 1 µm. Here, the distance can be specified, for example, by a distance between opposite end portions of two adjacent layers among the organic compound layers 103R, 103G, and 103B. Reducing the distance between the island-shaped organic compound layers can provide a display apparatus having high resolution and a high aperture ratio. In addition, the distance between the first electrodes of adjacent light-emitting devices can also be shortened to be, for example, less than or equal to 10 µm, less than or equal to 8 µm, less than or equal to 5 µm, less than or equal to 3 µm, or less than or equal to 2 µm. Note that the distance between the first electrodes of adjacent light-emitting devices is preferably greater than or equal to 2 µm and less than or equal to 5 µm.

Next, as illustrated in FIG. 8A, the mask layers 159R, 159G, and 159B are preferably removed. The sacrificial layers 158R, 158G, and 158B and the mask layers 159R, 159G, and 159B remain in the display apparatus in some cases depending on the subsequent steps. Removing the mask layers 159R, 159G, and 159B at this stage can inhibit the mask layers 159R, 159G, and 159B from being left in the display apparatus. For example, in the case where a conductive material is used for the mask layers 159R, 159G, and 159B, removing the mask layers 159R, 159G, and 159B in advance can suppress generation of a leakage current, formation of a capacitor, and the like due to the remaining mask layers 159R, 159G, and 159B.

This embodiment shows an example where the mask layers 159R, 159G, and 159B are removed; however, it is possible that the mask layers 159R, 159G, and 159B are not removed. For example, in the case where the mask layers 159R, 159G, and 159B contain the above-described material having a property of blocking ultraviolet rays, the procedure preferably proceeds to the next step without removing the mask layers 159R, 159G, and 159B, in which case the organic compound layer can be protected from ultraviolet rays.

The step of removing the mask layers can be performed by a method similar to that for the step of processing the mask layers. Specifically, by using a wet etching method, damage applied to the organic compound layers 103R, 103G, and 103B at the time of removing the mask layers can be reduced as compared to the case of using a dry etching method.

The mask layers may be removed by being dissolved in a solvent such as water or an alcohol. Examples of an alcohol include ethyl alcohol, methyl alcohol, isopropyl alcohol (IPA), and glycerin.

After the mask layers are removed, drying treatment may be performed in order to remove water included in the organic compound layers 103R, 103G, and 103B and water adsorbed on the surfaces of the organic compound layers 103R, 103G, and 103B. For example, heat treatment in an inert gas atmosphere or a reduced-pressure atmosphere can be performed. The heat treatment can be performed at a substrate temperature of higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 8B, the inorganic insulating film 125f to be the inorganic insulating layer 125 is formed to cover the organic compound layers 103R, 103G, and 103B and the sacrificial layers 158R, 158G, and 158B.

As described later, an insulating film to be the insulating layer 127 is formed in contact with the top surface of the inorganic insulating film 125f. Therefore, the top surface of the inorganic insulating film 125f preferably has a high affinity for the material used for the insulating film (e.g., a photosensitive resin composition containing an acrylic resin). To improve the affinity, surface treatment is preferably performed so that the top surface of the inorganic insulating film 125f is made hydrophobic or its hydrophobic properties are improved. For example, it is preferable to perform the treatment using a silylation agent such as hexamethyldisilazane (HMDS). By making the top surface of the inorganic insulating film 125f hydrophobic in such a manner, the above insulating film 127f can be formed with favorable adhesion. Note that the above-described hydrophobization treatment may be performed as the surface treatment.

Then, as illustrated in FIG. 8C, an insulating film 127f to be the insulating layer 127 is formed over the inorganic insulating film 125f.

The inorganic insulating film 125f and the insulating film 127f are preferably formed by a formation method by which the organic compound layers 103R, 103G, and 103B are less damaged. The inorganic insulating film 125f, which is formed in contact with the side surfaces of the organic compound layers 103R, 103G, and 103B, is particularly preferably formed by a formation method that causes less damage to the organic compound layers 103R, 103G, and 103B than the method of forming the insulating film 127f.

Each of the insulating films 125f and 127f is formed at a temperature lower than the upper temperature limit of the organic compound layers 103R, 103G, and 103B. When the insulating film 125f is formed at a high substrate temperature, the formed insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness of greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case deposition damage is reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher deposition rate than an ALD method. In that case, a highly reliable display apparatus can be manufactured with high productivity.

The insulating film 127f is preferably formed by the aforementioned wet process. The insulating film 127f is preferably formed by spin coating using a photosensitive material, for example, and specifically preferably formed using a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limit of the organic compound layers 103R, 103G, and 103B. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent contained in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are sandwiched between any two of the conductive layers 152R, 152G, and 152B and around the conductive layer 152C. Thus, the top surfaces of the conductive layers 152R, 152G, 152B, and 152C are irradiated with visible light or ultraviolet rays. Note that when a negative photosensitive material is used for the insulating film 127f, the region where the insulating layer 127 is to be formed is irradiated with visible light or ultraviolet rays.

The width of the insulating layer 127 formed later can be controlled in accordance with the exposed region of the insulating film 127f. In this embodiment, processing is performed such that the insulating layer 127 includes a portion overlapping the top surface of the conductive layer 151.

Light used for exposure preferably includes the i-line (wavelength: 365 nm). Furthermore, light used for exposure may include at least one of the g-line (wavelength: 436 nm) and the h-line (wavelength: 405 nm).

Here, when a barrier insulating layer against oxygen (e.g., an aluminum oxide film) is provided as one or both of the sacrificial layer 158 (the sacrificial layers 158R, 158G, and 158B) and the inorganic insulating film 125f, diffusion of oxygen to the organic compound layers 103R, 103G, and 103B can be suppressed. When the organic compound layer is irradiated with light (visible light or ultraviolet rays), the organic compound contained in the organic compound layer is brought into an excited state and a reaction between the organic compound and oxygen in the atmosphere is promoted in some cases. Specifically, when the organic compound layer is irradiated with light (visible light or ultraviolet rays) in an atmosphere including oxygen, oxygen might be bonded to the organic compound contained in the organic compound layer. By providing the sacrificial layer 158 and the inorganic insulating film 125f over the island-shaped organic compound layer, bonding of oxygen in the atmosphere to the organic compound contained in the organic compound layer can be suppressed.

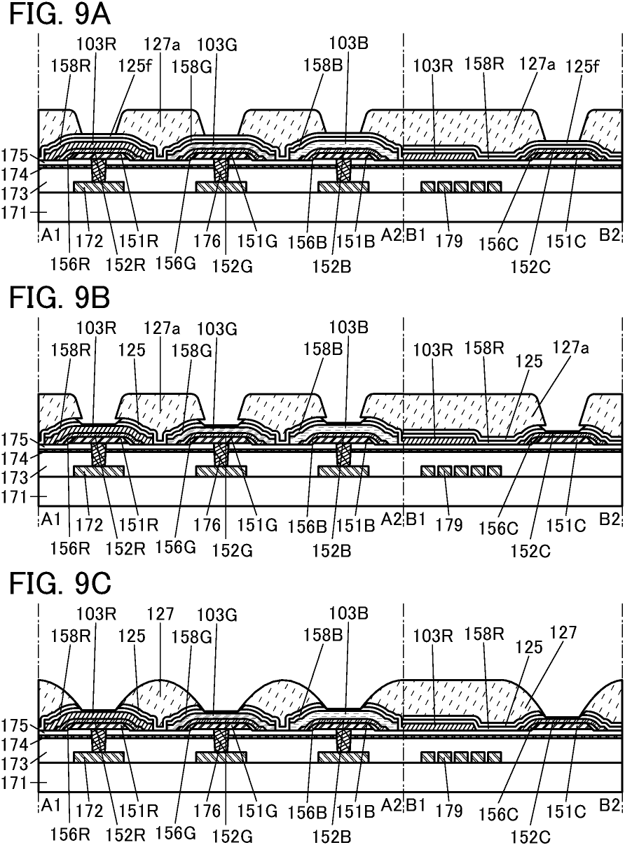

Next, as illustrated in FIG. 9A, development is performed to remove the exposed region of the insulating film 127f, whereby an insulating layer 127a is formed. The insulating layer 127a is formed in regions that are sandwiched between any two of the conductive layers 152R, 152G, and 152B and a region surrounding the conductive layer 152C. Here, when an acrylic resin is used for the insulating film 127f, an alkaline solution, such as TMAH, can be used as a developer.

Then, a residue (scum) due to the development may be removed. For example, the residue can be removed by ashing using oxygen plasma.

Etching may be performed so that the surface level of the insulating layer 127a is adjusted. The insulating layer 127a may be processed by ashing using oxygen plasma, for example. In the case where a non-photosensitive material is used for the insulating film 127f, the surface level of the insulating film 127f can be adjusted by the ashing, for example.

Next, as illustrated in FIG. 9B, etching treatment is performed with the insulating layer 127a as a mask to remove part of the inorganic insulating film 125f and reduce the thickness of part of the sacrificial layers 158R, 158G, and 158B. Thus, the inorganic insulating layer 125 is formed under the insulating layer 127a. Moreover, the surfaces of the thin portions in the sacrificial layers 158R, 158G, and 158B are exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that of the sacrificial layers 158R, 158G, and 158B, in which case the first etching treatment can be performed concurrently.

By etching using the insulating layer 127a with a tapered side surface as a mask, the side surface of the inorganic insulating layer 125 and upper end portions of the side surfaces of the sacrificial layers 158R, 158G, and 158B can be made to have a tapered shape relatively easily.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158R, 158G, and 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used. The capacitively coupled plasma etching apparatus including parallel plate electrodes may have a structure in which a high-frequency voltage is applied to one of the parallel plate electrodes. Alternatively, the capacitively coupled plasma etching apparatus may have a structure in which different high-frequency voltages are applied to one of the parallel-plate electrodes. Alternatively, the capacitively coupled plasma etching apparatus may have a structure in which high-frequency voltages with the same frequency are applied to the parallel-plate electrodes. Alternatively, the capacitively coupled plasma etching apparatus may have a structure in which high-frequency voltages with different frequencies are applied to the parallel-plate electrodes.

In the case of performing dry etching, a by-product or the like generated by the dry etching might be deposited on the top surface and the side surface of the insulating layer 127a, for example. Accordingly, a constituent of the etching gas, a constituent of the inorganic insulating film 125f, a constituent of the sacrificial layers 158R, 158G, and 158B, and the like might be included in the insulating layer 127 in the completed display apparatus.

The first etching treatment is preferably performed by wet etching. The use of a wet etching method can reduce damage to the organic compound layers (the organic compound layers 103R, 103G, and 103B), as compared to the case of using a dry etching method. For example, the wet etching can be performed using an alkaline solution. For instance, TMAH, which is an alkaline solution, can be used for the wet etching of an aluminum oxide film. In this case, puddle wet etching can be performed. Note that the inorganic insulating film 125f is preferably formed using a material similar to that of the sacrificial layers 158R, 158G, and 158B, in which case the above etching treatment can be performed concurrently.

The sacrificial layers 158R, 158G, and 158B are not removed completely by the first etching treatment, and the etching treatment is stopped when the thickness of the sacrificial layers 158R, 158G, and 158B is reduced. The corresponding sacrificial layers 158R, 158G, and 158B remain over the organic compound layers 103R, 103G, and 103B in this manner, whereby the organic compound layers 103R, 103G, and 103B can be prevented from being damaged by treatment in a later step.

Next, light exposure is preferably performed on the entire substrate so that the insulating layer 127a is irradiated with visible light or ultraviolet rays. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a to a tapered shape.

Here, when a barrier insulating layer against oxygen (e.g., an aluminum oxide film) exists as each of the sacrificial layers 158R, 158G, and 158B, diffusion of oxygen to the organic compound layers 103R, 103G, and 103B can be suppressed. When the organic compound layer is irradiated with light (visible light or ultraviolet rays), the organic compound contained in the organic compound layer is brought into an excited state and a reaction between the organic compound and oxygen in the atmosphere is promoted in some cases. Specifically, when the organic compound layer is irradiated with light (visible light or ultraviolet rays) in an atmosphere including oxygen, oxygen might be bonded to the organic compound contained in the organic compound layer. By providing the sacrificial layers 158R, 158G, and 158B over the island-shaped organic compound layer, bonding of oxygen in the atmosphere to the organic compound contained in the organic compound layer can be suppressed.

Then, heat treatment (also referred to as post-baking) is performed. The heat treatment can change the insulating layer 127a into the insulating layer 127 having a tapered side surface (FIG. 9C). The heat treatment is conducted at a temperature lower than the upper temperature limit of the organic compound layer. The heat treatment can be performed at a substrate temperature of higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be an air atmosphere or an inert gas atmosphere. Moreover, the heating atmosphere may be an atmospheric-pressure atmosphere or a reduced-pressure atmosphere. The substrate temperature in the heat treatment of this step is preferably higher than that in the heat treatment (prebaking) after the formation of the insulating film 127f. Accordingly, adhesion between the insulating layer 127 and the inorganic insulating layer 125 can be improved, and corrosion resistance of the insulating layer 127 can be increased.

When the sacrificial layers 158R, 158G, and 158B are not completely removed by the first etching treatment and the thinned sacrificial layers 158R, 158G, and 158B are left, the organic compound layers 103R, 103G, and 103B can be prevented from being damaged and deteriorating in the heat treatment. This increases the reliability of the light-emitting device.

Note that the side surface of the insulating layer 127 may have a concave shape depending on the material of the insulating layer 127 and the temperature, time, and atmosphere of the post-baking. For example, when the temperature of the post-baking is higher or the duration of the post-baking is longer, the shape of the insulating layer 127 is more likely to change and thus a concave shape may be more likely to be formed.

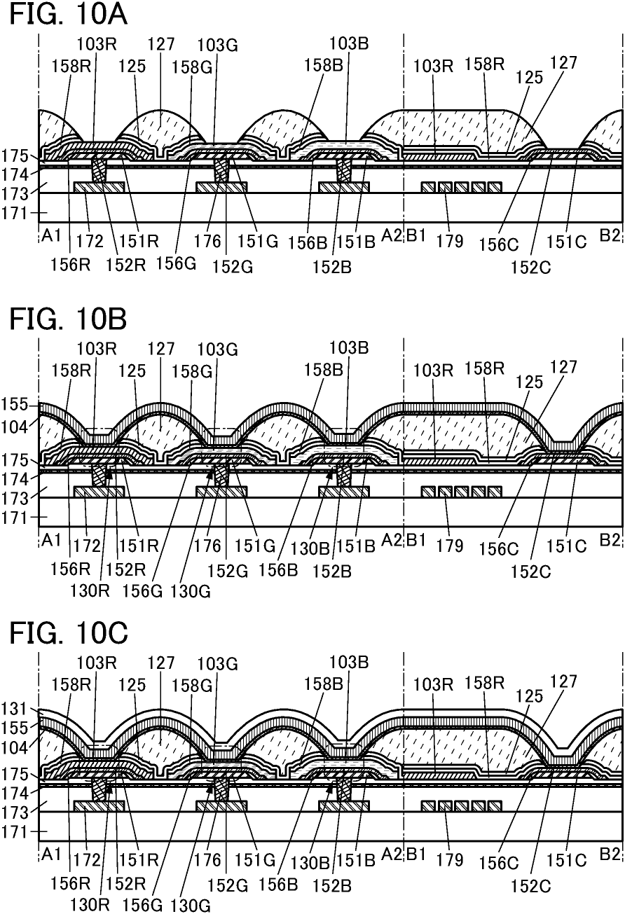

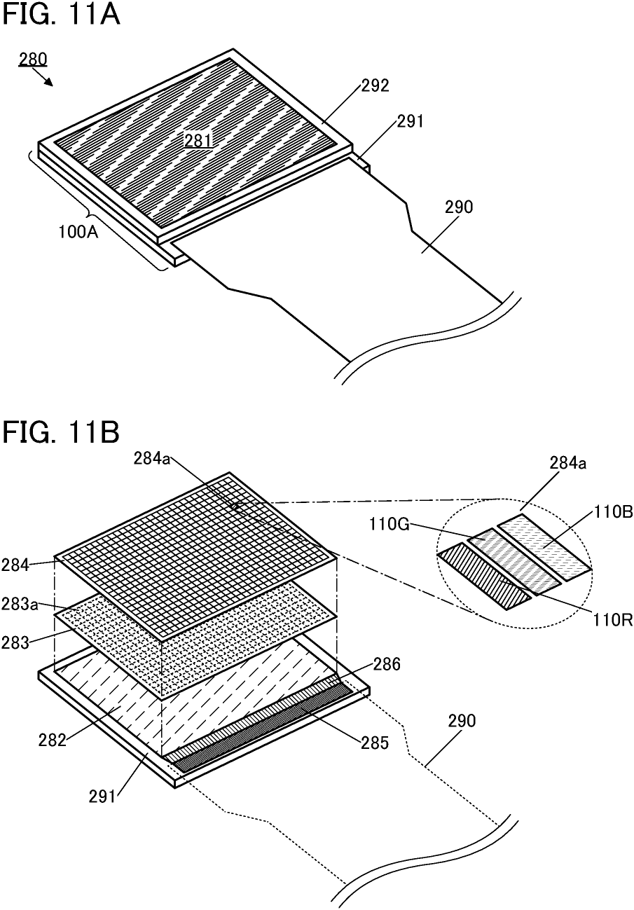

Next, as illustrated in FIG. 10A, etching treatment is performed with the insulating layer 127 as a mask to remove part of the sacrificial layers 158R, 158G, and 158B. Note that part of the inorganic insulating layer 125 is also removed in some cases. Thus, openings are formed in the sacrificial layers 158R, 158G, and 158B, and the top surfaces of the organic compound layers 103R, 103G, and 103B and the conductive layer 152C are exposed. Note that the etching treatment using the insulating layer 127 as a mask may be hereinafter referred to as second etching treatment.

The end portion of the inorganic insulating layer 125 is covered with the insulating layer 127. FIG. 10A illustrates an example in which part of the end portion of the sacrificial layer 158G (specifically a tapered portion formed by the first etching treatment) is covered with the insulating layer 127 and a tapered portion formed by the second etching treatment is exposed.

If the first etching treatment is not performed and the inorganic insulating layer 125 and the mask layer are collectively etched after the post-baking, the inorganic insulating layer 125 and the mask layer under the end portion of the insulating layer 127 may disappear because of side-etching and a void may be formed. The void causes unevenness on the formation surface of the common electrode 155, so that a step-cut is more likely to be caused in the common electrode 155. Even when a void is formed owing to side-etching of the inorganic insulating layer 125 and the mask layer by the first etching treatment, the post-baking performed subsequently can make the insulating layer 127 fill the void. After that, the thinned mask layer is etched by the second etching treatment; thus, the amount of side-etching decreases, a void is less likely to be formed, and even if a void is formed, it can be extremely small. Consequently, the formation surface of the common electrode 155 can be made flatter.

Note that the insulating layer 127 may cover the entire end portion of the sacrificial layer 158G. For example, the end portion of the insulating layer 127 may droop to cover the end portion of the sacrificial layer 158G. As another example, the end portion of the insulating layer 127 may be in contact with the top surface of at least one of the organic compound layers 103R, 103G, and 103B. As described above, when light exposure is not performed on the insulating layer 127a after the development, the shape of the insulating layer 127 may be likely to change.

The second etching treatment is performed by wet etching. The use of a wet etching method can reduce damage to the organic compound layers 103R, 103G, and 103B, as compared to the case of using a dry etching method. The wet etching can be performed using an alkaline solution such as TMAH, for example.

Meanwhile, in the case where the second etching treatment is performed by a wet etching method and gaps due to, for example, poor adhesion between the organic compound layer 103 and another layer exist at the interface between the organic compound layer 103 and the sacrificial layer 158, the interface between the organic compound layer 103 and the inorganic insulating layer 125, and the interface between the organic compound layer 103 and the insulating layer 175, the chemical solution used in the second etching treatment sometimes enters the gaps to come into contact with the pixel electrode. Here, when the chemical solution comes into contact with both the conductive layer 151 and the conductive layer 152, one of the conductive layers 151 and 152 that has a lower spontaneous potential than the other suffers from galvanic corrosion in some cases. For example, when the conductive layer 151 is formed using aluminum and the conductive layer 152 is formed using indium tin oxide, the conductive layer 152 sometimes corrodes. As a result, the yield of the display apparatus decreases in some cases. Moreover, the reliability of the display apparatus is lowered in some cases.

The conductive layer 152, which covers the top and side surfaces of the conductive layer 151 as described above, can prevent the chemical solution from coming into contact with the conductive layer 151 in the second etching treatment even when gaps exist at the interface between the organic compound layer 103 and the sacrificial layer 158, the interface between the organic compound layer 103 and the inorganic insulating layer 125, and the interface between the organic compound layer 103 and the insulating layer 175. Thus, corrosion of the pixel electrode, e.g., the conductive layer 152, can be prevented.

Furthermore, when the insulating layer 156 is formed to include a region overlapping with the side surface of the conductive layer 151 and the conductive layer 152 is formed to cover the conductive layer 151 and the insulating layer 156, the step disconnection can be prevented, whereby the chemical solution can be prevented from coming into contact with the conductive layer 151 in the second etching treatment, for example. Thus, corrosion of the pixel electrode, e.g., the conductive layer 152, can be prevented.

As described above, by providing the insulating layer 127, the inorganic insulating layer 125, and the sacrificial layers 158R, 158G, and 158B, poor connection due to a disconnected portion and an increase in electric resistance due to a locally thinned portion can be inhibited from occurring in the common electrode 155 between the light-emitting devices. Thus, the display apparatus of one embodiment of the present invention can have improved display quality.

Heat treatment is performed after the organic compound layers 103R, 103G, and 103B are partly exposed. By the heat treatment, water included in the organic compound layers and water adsorbed on the surfaces of the organic compound layers, for example, can be removed. The shape of the insulating layer 127 may be changed by the heat treatment. Specifically, the insulating layer 127 may be widened to cover at least one of the end portion of the inorganic insulating layer 125, the end portions of the sacrificial layers 158R, 158G, and 158B, and the top surfaces of the organic compound layers 103R, 103G, and 103B.

If the temperature of the heat treatment is too low, water included in the organic compound layers and water adsorbed on the surface of the organic compound layers, for example, cannot be sufficiently removed. If the temperature of the heat treatment is too high, the organic compound layer 103 might deteriorate and the insulating layer 127 might change in shape excessively. Therefore, the temperature of the heat treatment is preferably higher than the temperature at which water is released from the organic compound layer 103 and lower than the glass transition temperature of an organic compound included in the organic compound layer 103, further preferably lower than the glass transition temperature of an organic compound included in the upper surface of the organic compound layer 103. Specifically, the substrate temperature is higher than or equal to 80° C. and lower than or equal to 130° C., preferably higher than or equal to 90° C. and lower than or equal to 120° C., further preferably higher than or equal to 100° C. and lower than or equal to 120° C., further preferably higher than or equal to 100° C. and lower than or equal to 110° C. The heating atmosphere may be an air atmosphere or an inert gas atmosphere. Although the heating atmosphere may be an atmospheric-pressure atmosphere or a reduced-pressure atmosphere, a reduced-pressure atmosphere is preferred to prevent re-adsorption of water released from the organic compound layer 103.

By the heat treatment, water included in the organic compound layers and water adsorbed on the surface of the organic compound layers, for example, can be sufficiently removed without deterioration of the organic compound layers 103R, 103G, and 103B and an excessive change in the shape of the insulating layer 127. Thus, degradation of the characteristics of the light-emitting device can be prevented.

Next, as illustrated in FIG. 10B, the common layer 104 and the common electrode 155 are formed over the organic compound layers 103R, 103G, and 103B, the conductive layer 152C, and the insulating layer 127. The common layer 104 and common electrode 155 can be formed by a sputtering method, a vacuum evaporation method, or the like. The common layer 104 may be formed by an evaporation method while the common electrode 155 may be formed by a sputtering method.

Next, as illustrated in FIG. 10C, the protective layer 131 is formed over the common electrode 155. The protective layer 131 can be formed by a vacuum evaporation method, a sputtering method, a CVD method, an ALD method, or the like.

Then, the substrate 120 is bonded over the protective layer 131 using the resin layer 122, whereby the display apparatus can be manufactured. In the method for manufacturing the display apparatus of one embodiment of the present invention, the insulating layer 156 is formed to include a region overlapping the side surface of the conductive layer 151 and the conductive layer 152 is formed to cover the conductive layer 151 and the insulating layer 156 as described above. This can increase the yield of the display apparatus and inhibit generation of defects.

As described above, in the method for manufacturing the display apparatus of one embodiment of the present invention, the island-shaped organic compound layers 103R, 103G, and 103B are formed not by using a fine metal mask but by processing a film formed on the entire surface; thus, the island-shaped layers can be formed to have a uniform thickness. Consequently, a high-resolution display apparatus or a display apparatus with a high aperture ratio can be obtained. Furthermore, even when the resolution or the aperture ratio is high and the distance between the subpixels is extremely short, the organic compound layers 103R, 103G, and 103B can be inhibited from being in contact with each other in the adjacent subpixels. As a result, generation of a leakage current between the subpixels can be inhibited. This can prevent crosstalk, so that a display apparatus with extremely high contrast can be obtained. Moreover, even a display apparatus that includes tandem light-emitting devices formed by a photolithography technique can have favorable characteristics.

### Embodiment 4

In this embodiment, a display apparatus of one embodiment of the present invention will be described.

The display apparatus in this embodiment can be a high-resolution display apparatus. Thus, the display apparatus in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display apparatus in this embodiment can be a high-definition display apparatus or a large-sized display apparatus. Accordingly, the display apparatus in this embodiment can be used for display portions of a digital camera, a digital video camera, a digital photo frame, a mobile phone, a portable game console, a portable information terminal, and an audio reproducing device, in addition to display portions of electronic devices with a relatively large screen, such as a television device, desktop and notebook personal computers, a monitor of a computer and the like, digital signage, and a large game machine such as a pachinko machine.

### [Display Module]

FIG. 11A is a perspective view of a display module 280. The display module 280 includes a display apparatus 100A and an FPC 290.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 11B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapped by the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 11B. The pixels 284a can employ any of the structures described in the above embodiments. FIG. 11B illustrates an example where the pixel 284a has a structure similar to that of the pixel 178 illustrated in FIGS. 3A and 3B.

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor for one light-emitting device. A gate signal is input to a gate of the selection transistor, and a video signal is input to a source or a drain of the selection transistor. With such a structure, an active-matrix display apparatus is achieved.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be greater than or equal to 40% and less than 100%, preferably greater than or equal to 50% and less than or equal to 95%, further preferably greater than or equal to 60% and less than or equal to 95%. Furthermore, the pixels 284a can be arranged extremely densely and thus the display portion 281 can have significantly high resolution. For example, the pixels 284a are preferably arranged in the display portion 281 with a resolution of greater than or equal to 2000 ppi, further preferably greater than or equal to 3000 ppi, still further preferably greater than or equal to 5000 ppi, yet still further preferably greater than or equal to 6000 ppi, and less than or equal to 20000 ppi or less than or equal to 30000 ppi.

Such a display module 280 has extremely high resolution, and thus can be suitably used for a VR device such as a HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic devices including a relatively small display portion. For example, the display module 280 can be favorably used in a display portion of a wearable electronic device, such as a wrist watch.

### [Display Apparatus 100A]

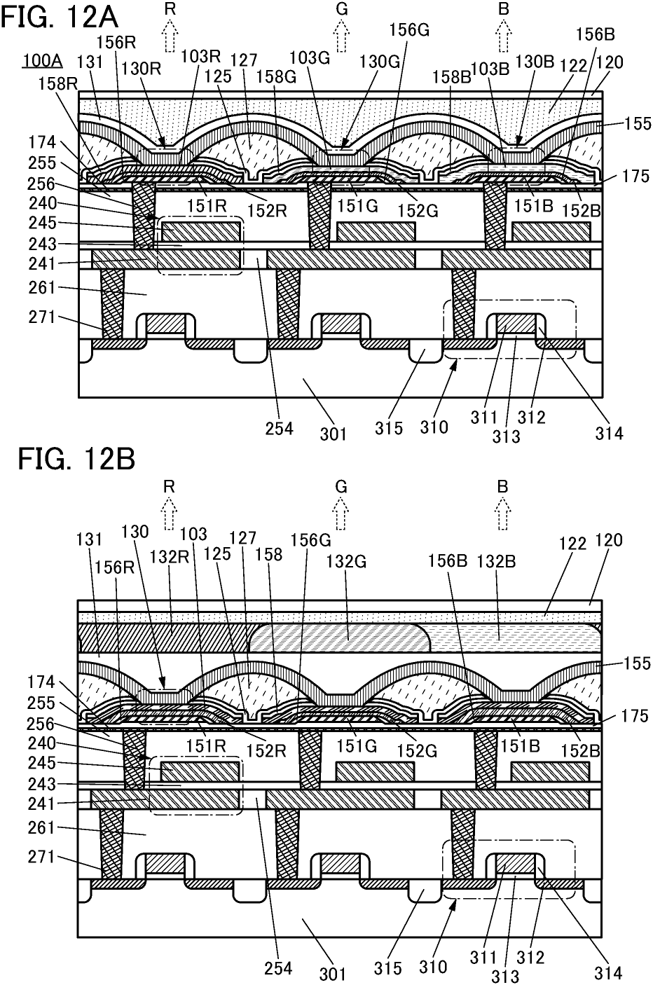

The display apparatus 100A illustrated in FIG. 12A includes a substrate 301, the light-emitting devices 130R, 130G, and 130B, a capacitor 240, and a transistor 310.

The substrate 301 corresponds to the substrate 291 in FIGS. 11A and 11B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. FIG. 12A illustrates an example in which the light-emitting devices 130R, 130G, and 130B each have the stacked-layer structure illustrated in FIG. 6A. An insulator is provided in regions between adjacent light-emitting devices. For example, in FIG. 12A, the inorganic insulating layer 125 and the insulating layer 127 over the inorganic insulating layer 125 are provided in those regions.

The insulating layer 156R is provided to include a region overlapping the side surface of the conductive layer 151R of the light-emitting device 130R. The insulating layer 156G is provided to include a region overlapping the side surface of the conductive layer 151G of the light-emitting device 130G. The insulating layer 156B is provided to include a region overlapping the side surface of the conductive layer 151B of the light-emitting device 130B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R of the light-emitting device 130R. The sacrificial layer 158G is positioned over the organic compound layer 103G of the light-emitting device 130G. The sacrificial layer 158B is positioned over the organic compound layer 103B of the light-emitting device 130B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The protective layer 131 is provided over the light-emitting devices 130R, 130G, and 130B. The substrate 120 is bonded to the protective layer 131 with the resin layer 122. Embodiment 3 can be referred to for the details of the light-emitting device 130 and the components thereover up to the substrate 120. The substrate 120 corresponds to the substrate 292 in FIG. 11A.

FIG. 12B illustrates a variation example of the display apparatus 100A illustrated in FIG. 12A. The display apparatus illustrated in FIG. 12B includes the coloring layers 132R, 132G, and 132B, and each of the light-emitting devices 130 includes a region overlapped by one of the coloring layers 132R, 132G, and 132B. In the display apparatus illustrated in FIG. 12B, the light-emitting device 130 can emit white light, for example. For example, the coloring layer 132R, the coloring layer 132G, and the coloring layer 132B can transmit red light, green light, and blue light, respectively.

This embodiment can be combined as appropriate with the other embodiments or the examples. In this specification, in the case where a plurality of structure examples are shown in one embodiment, the structure examples can be combined as appropriate.

### Embodiment 5

In this embodiment, electronic devices of embodiments of the present invention will be described.

Electronic devices of this embodiment include the display apparatus of one embodiment of the present invention in their display portions. The display apparatus of one embodiment of the present invention is highly reliable and can be easily increased in resolution and definition. Thus, the display apparatus of one embodiment of the present invention can be used for display portions of a variety of electronic devices.

Examples of the electronic devices include a digital camera, a digital video camera, a digital photo frame, a mobile phone, a portable game console, a portable information terminal, and an audio reproducing device, in addition to electronic devices with a relatively large screen, such as a television device, desktop and notebook personal computers, a monitor of a computer and the like, digital signage, and a large game machine such as a pachinko machine.

In particular, the display apparatus of one embodiment of the present invention can have high resolution, and thus can be favorably used for an electronic device having a relatively small display portion. Examples of such an electronic device include watch-type and bracelet-type information terminal devices (wearable devices) and wearable devices worn on the head, such as a VR device like a head-mounted display, a glasses-type AR device, and an MR device.

The definition of the display apparatus of one embodiment of the present invention is preferably as high as HD (number of pixels: 1280 × 720), FHD (number of pixels: 1920 × 1080), WQHD (number of pixels: 2560 × 1440), WQXGA (number of pixels: 2560 × 1600), 4K (number of pixels: 3840 × 2160), or 8K (number of pixels: 7680 × 4320). In particular, definition of 4K, 8K, or higher is preferable. The pixel density (resolution) of the display apparatus of one embodiment of the present invention is preferably higher than or equal to 100 ppi, further preferably higher than or equal to 300 ppi, further preferably higher than or equal to 500 ppi, further preferably higher than or equal to 1000 ppi, still further preferably higher than or equal to 2000 ppi, still further preferably higher than or equal to 3000 ppi, still further preferably higher than or equal to 5000 ppi, yet further preferably higher than or equal to 7000 ppi. With such a display apparatus having one or both of high definition and high resolution, the electronic device can provide higher realistic sensation, sense of depth, and the like in personal use such as portable use or home use. There is no particular limitation on the screen ratio (aspect ratio) of the display apparatus of one embodiment of the present invention. For example, the display apparatus is compatible with a variety of screen ratios such as 1:1 (a square), 4:3, 16:9, and 16:10.

The electronic device in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic device in this embodiment can have a variety of functions. For example, the electronic device in this embodiment can have a function of displaying a variety of data (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

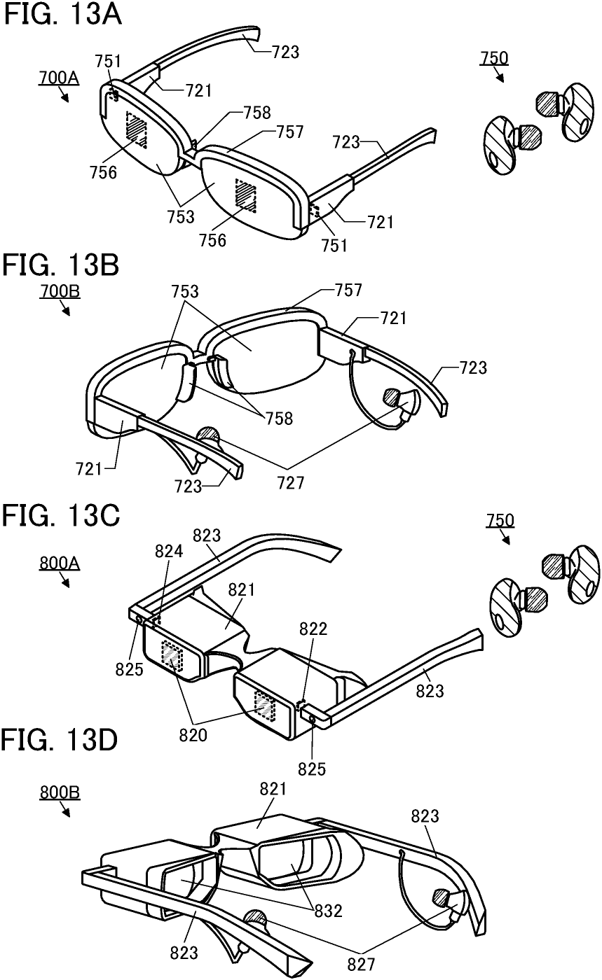

Examples of head-mounted wearable devices are described with reference to FIGS. 13A to 13D. These wearable devices have at least one of a function of displaying AR contents, a function of displaying VR contents, a function of displaying SR contents, and a function of displaying MR contents. The electronic device having a function of displaying contents of at least one of AR, VR, SR, MR, and the like enables the user to feel a higher level of immersion.

An electronic device 700A illustrated in FIG. 13A and an electronic device 700B illustrated in FIG. 13B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display apparatus of one embodiment of the present invention can be used for the display panels 751. Thus, a highly reliable electronic device is obtained.

The electronic devices 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic devices 700A and 700B are electronic devices capable of AR display.

In the electronic devices 700A and 700B, a camera capable of capturing images of the front side may be provided as the image capturing portion. Furthermore, when the electronic devices 700A and 700B are provided with an acceleration sensor such as a gyroscope sensor, the orientation of the user’s head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic devices 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a video can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic device 800A illustrated in FIG. 13C and an electronic device 800B illustrated in FIG. 13D each include a pair of display portions 820, a housing 821, a communication portion 822, a pair of wearing portions 823, a control portion 824, a pair of image capturing portions 825, and a pair of lenses 832.

The display apparatus of one embodiment of the present invention can be used in the display portions 820. Thus, a highly reliable electronic device is obtained.

The display portions 820 are provided at positions where the user can see through the lenses 832 inside the housing 821. When the pair of display portions 820 display different images, three-dimensional display using parallax can be performed.

The electronic devices 800A and 800B can be regarded as electronic devices for VR. The user who wears the electronic device 800A or the electronic device 800B can see images displayed on the display portions 820 through the lenses 832.

The electronic devices 800A and 800B preferably include a mechanism for adjusting the lateral positions of the lenses 832 and the display portions 820 so that the lenses 832 and the display portions 820 are positioned optimally in accordance with the positions of the user’s eyes. Moreover, the electronic devices 800A and 800B preferably include a mechanism for adjusting focus by changing the distance between the lenses 832 and the display portions 820.

The electronic device 800A or the electronic device 800B can be mounted on the user’s head with the wearing portions 823. FIG. 13C, for instance, shows an example where the wearing portion 823 has a shape like a temple (also referred to as a joint or the like) of glasses; however, one embodiment of the present invention is not limited thereto. The wearing portion 823 can have any shape with which the user can wear the electronic device, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

Although an example where the image capturing portions 825 are provided is shown here, a range sensor (hereinafter also referred to as a sensing portion) capable of measuring a distance between the user and an object just needs to be provided. In other words, the image capturing portion 825 is one embodiment of the sensing portion. As the sensing portion, an image sensor or a range image sensor such as a light detection and ranging (LiDAR) sensor can be used, for example. By using images obtained by the camera and images obtained by the range image sensor, more information can be obtained and a gesture operation with higher accuracy is possible.

The electronic device 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic device 800A.

The electronic devices 800A and 800B may each include an input terminal. To the input terminal, a cable for supplying a video signal from a video output device or the like, power for charging a battery provided in the electronic device, and the like can be connected.

The electronic device of one embodiment of the present invention may have a function of performing wireless communication with earphones 750. The earphones 750 include a communication portion (not illustrated) and has a wireless communication function. The earphones 750 can receive information (e.g., audio data) from the electronic device with the wireless communication function. For example, the electronic device 700A in FIG. 13A has a function of transmitting information to the earphones 750 with the wireless communication function. As another example, the electronic device 800A in FIG. 13C has a function of transmitting information to the earphones 750 with the wireless communication function.

The electronic device may include an earphone portion. The electronic device 700B in FIG. 13B includes earphone portions 727. For example, the earphone portion 727 can be connected to the control portion by wire. Part of a wiring that connects the earphone portion 727 and the control portion may be positioned inside the housing 721 or the wearing portion 723.

Similarly, the electronic device 800B in FIG. 13D includes earphone portions 827. For example, the earphone portion 827 can be connected to the control portion 824 by wire. Part of a wiring that connects the earphone portion 827 and the control portion 824 may be positioned inside the housing 821 or the wearing portion 823. Alternatively, the earphone portions 827 and the wearing portions 823 may include magnets. This is preferred because the earphone portions 827 can be fixed to the wearing portions 823 with magnetic force and thus can be easily housed.

The electronic device may include an audio output terminal to which earphones, headphones, or the like can be connected. The electronic device may include one or both of an audio input terminal and an audio input mechanism. As the audio input mechanism, a sound collecting device such as a microphone can be used, for example. The electronic device may have a function of a headset by including the audio input mechanism.

As described above, both the glasses-type device (e.g., the electronic devices 700A and 700B) and the goggles-type device (e.g., the electronic devices 800A and 800B) are preferable as the electronic device of one embodiment of the present invention.

The electronic device of one embodiment of the present invention can transmit information to earphones by wire or wirelessly.

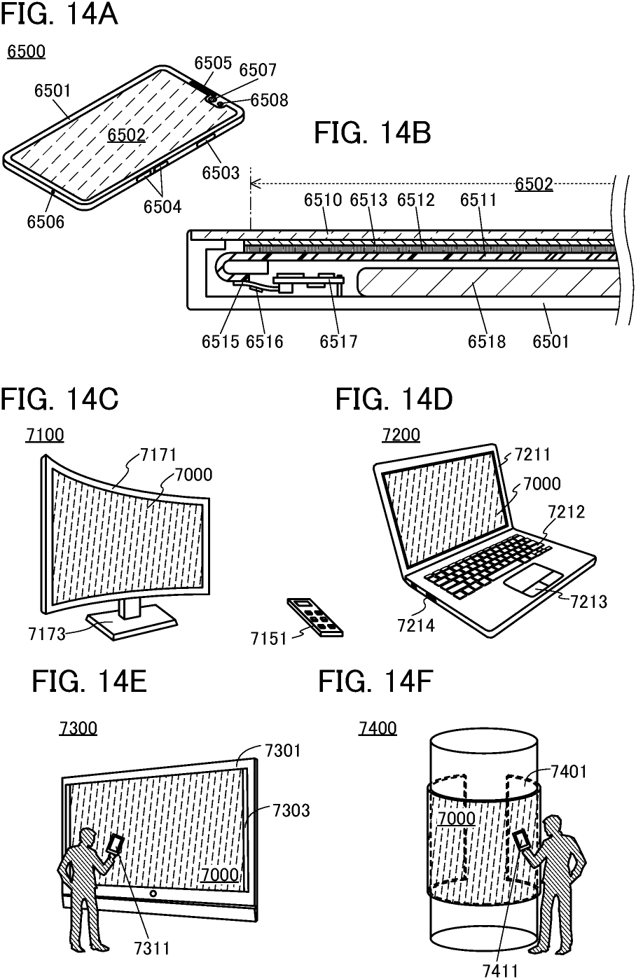

An electronic device 6500 illustrated in FIG. 14A is a portable information terminal that can be used as a smartphone.

The electronic device 6500 includes a housing 6501, a display portion 6502, a power button 6503, buttons 6504, a speaker 6505, a microphone 6506, a camera 6507, a light source 6508, and the like. The display portion 6502 has a touch panel function.

The display apparatus of one embodiment of the present invention can be used in the display portion 6502. Thus, a highly reliable electronic device is obtained.

FIG. 14B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

A flexible display of one embodiment of the present invention can be used in the display panel 6511. Thus, an extremely lightweight electronic device can be achieved. Since the display panel 6511 is extremely thin, the battery 6518 with high capacity can be mounted without an increase in the thickness of the electronic device. Moreover, part of the display panel 6511 is folded back so that a connection portion with the FPC 6515 is provided on the back side of the pixel portion, whereby an electronic device with a narrow bezel can be achieved.

FIG. 14C illustrates an example of a television device. In a television device 7100, a display portion 7000 is incorporated in a housing 7171. Here, the housing 7171 is supported by a stand 7173.

The display apparatus of one embodiment of the present invention can be used in the display portion 7000. Thus, a highly reliable electronic device is obtained.

Operation of the television device 7100 illustrated in FIG. 14C can be performed with an operation switch provided in the housing 7171 and a separate remote controller 7151. Alternatively, the display portion 7000 may include a touch sensor, and the television device 7100 may be operated by touch on the display portion 7000 with a finger or the like. The remote controller 7151 may be provided with a display portion for displaying information output from the remote controller 7151. With operation keys or a touch panel of the remote controller 7151, channels and volume can be controlled and images displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 14D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display apparatus of one embodiment of the present invention can be used in the display portion 7000. Thus, a highly reliable electronic device is obtained.

FIGS. 14E and 14F illustrate examples of digital signage.

Digital signage 7300 illustrated in FIG. 14E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 14F shows digital signage 7400 attached to a cylindrical pillar 7401. The digital signage 7400 includes the display portion 7000 provided along a curved surface of the pillar 7401.

In FIGS. 14E and 14F, the display apparatus of one embodiment of the present invention can be used in the display portion 7000. Thus, a highly reliable electronic device is obtained.

A larger area of the display portion 7000 can increase the amount of information that can be provided at a time. The display portion 7000 having a larger area attracts more attention, so that the effectiveness of the advertisement can be increased, for example.

The touch panel is preferably used in the display portion 7000, in which case in addition to display of still or moving images on the display portion 7000, intuitive operation by a user is possible. Moreover, in the case of an application for providing information such as route information or traffic information, usability can be enhanced by intuitive operation.

As illustrated in FIGS. 14E and 14F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

This embodiment can be combined as appropriate with the other embodiments or the examples. In this specification, in the case where a plurality of structure examples are shown in one embodiment, the structure examples can be combined as appropriate.

### Example 1

### (Synthesis Example 1)

In this example, physical properties and a synthesis method of the organic compound of one embodiment of the present invention are described. Specifically, a synthesis method of 8,8′-pyridine-2,6-diyl-bis(5,6,7,8-tetrahydroimidazo[1,2-a]pyrimidine) (abbreviation: 2,6tip2Py) represented by Structural Formula (100) in Embodiment 1 is described. The structure of 2,6tip2Py is shown below.

### [Chemical Formula 15]

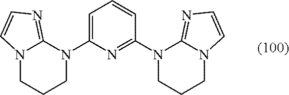 [SMILES: c1cc(N2CCCn3ccnc32)nc(N2CCCn3ccnc32)c1]

### <Synthesis of 2,6tip2Py>

Into a 200 mL three-neck flask were put 1.3 g (5.5 mmol) of 2,6-dibromopyridine, 1.7 g (16 mmol) of potassium-tert-butoxide (abbreviation: KOtBu), 0.21 g (0.33 mmol) of (±)-2,2′-bis(diphenylphosphino)-1,1′-binaphthyl (abbreviation: rac-BINAP), and 50 mg (0.22 mol) of palladium acetate (abbreviation: Pd(OAc)₂). To this mixture was added 1.5 g (12 mmol) of 5,6,7,8-tetrahydroimidazo[1,2-a]pyrimidine, and the air in the flask was replaced with nitrogen. To this mixture was added 19 mL of dehydrated toluene, the mixture in the flask was degassed under reduced pressure, and the air in the flask was replaced with nitrogen. This mixture was stirred for 8 hours while heated at 90° C., and then the mixture was cooled down to room temperature. After the reaction was completed, this reaction mixture was suction filtrated to give a residue. Ethyl acetate was added to the obtained solid, and the mixture was stirred while heated at 70° C. for 2 hours. Then, an insoluble material was removed by suction filtration, and the filtrate was concentrated under reduced pressure. The obtained solid was recrystallized with a mixed solvent of ethyl acetate and hexane to give 1.1 g of a gray solid in a 64% yield. The synthesis scheme of 2,6tip2Py is shown in Formula (a-1) below.

### [Chemical Formula 16]

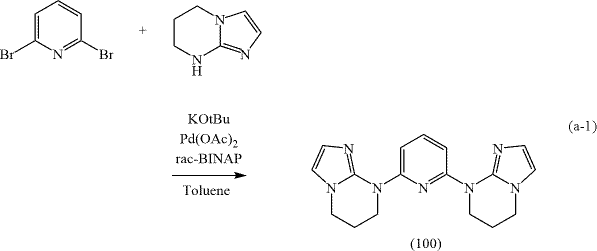

By a train sublimation method, 1.1 g of the obtained gray solid was purified by heating at 190° C. for 24 hours under a pressure of 2.9 Pa with an argon flow rate of 5 mL/min. As a result, 0.64 g of the target white solid was obtained at a 56% collection rate.

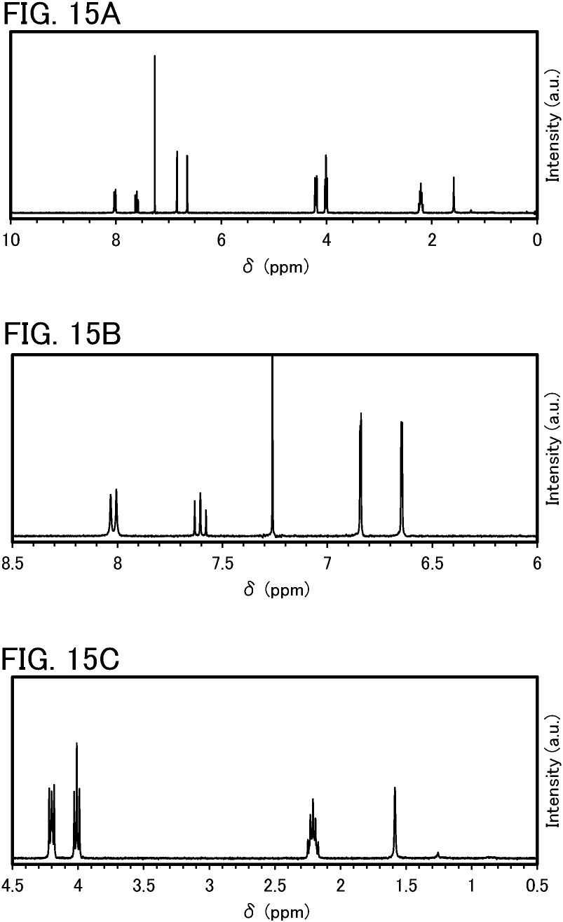

FIGS. 15A to 15C show a nuclear magnetic resonance (¹H-NMR) spectrum of 2,6tip2Py after the purification by sublimation. Results of ¹H NMR measurements are shown below. The results reveal that 2,6tip2Py was obtained.

¹H NMR (CDCl₃, 300 MHz): δ = 8.02 (d, J = 8.1 Hz, 2H), 7.61 (t, J = 8.1 Hz, 1H), 6.84 (d, J = 1.5 Hz, 2H), 6.65 (d, J = 1.5 Hz, 2H), 4.22-4.18 (m, 4H), 4.01 (t, J = 6.0 Hz, 4H), 2.26-2.16 (m, 4H).

### <Measurement of Emission and Absorption Spectra>

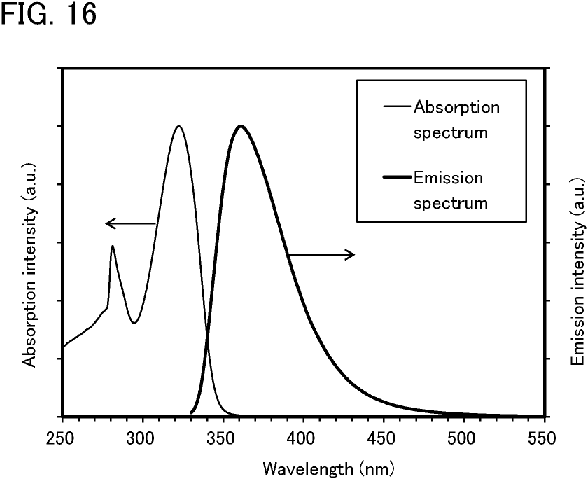

An ultraviolet-visible absorption spectrum (hereinafter, simply referred to as an absorption spectrum) of 2,6tip2Py in a toluene solution and an emission spectrum thereof were measured. The absorption spectrum was measured with an ultraviolet-visible spectrophotometer (V-770, produced by JASCO Corporation). The emission spectrum was measured with a fluorescence spectrophotometer (FP-8600, manufactured by JASCO Corporation). FIG. 16 shows the obtained absorption and emission spectra of 2,6tip2Py in the toluene solution. The horizontal axis represents the wavelength and the vertical axes represent the absorption intensity and the emission intensity.

As shown in FIG. 16, in the case of 2,6tip2Py in the toluene solution, an absorption peak was observed at around 324 nm, and an emission peak was observed at around 361 nm.

### Example 2

### (Synthesis Example 2)

In this example, a synthesis method of 8,8′-(9,9′-spirobi[9H-fluorene]-2,7-diyl)bls(5,6,7,8-tetrahydrolmidazo[1,2-a]pyrimidine) (abbreviation: 2,7tip2SF) represented by Structural Formula (101) in Embodiment 1 is described. The structure of 2,7tip2SF is shown below.

### [Chemical Formula 17]

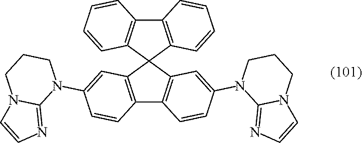 [SMILES: c1ccc2c(c1)-c1ccccc1C21c2cc(N3CCCn4ccnc43)ccc2-c2ccc(N3CCCn4ccnc43)cc21]

### <Synthesis of 2,7tip2SF>

Into a 200 mL three-neck flask were put 2.6 g (5.5 mmol) of 2,7-dibromo-9,9′-spirobi[9H-fluorene], 1.7 g (16 mmol) of potassium-tert-butoxide (abbreviation: KOtBu), 0.20 g (0.33 mmol) of (±)-2,2′-bis(diphenylphosphino)-1,1′-binaphthyl (abbreviation: rac-BINAP), and 52 mg (0.22 mol) of palladium acetate (abbreviation: Pd(OAc)₂). To this mixture was added 1.5 g (12 mmol) of 5,6,7,8-tetrahydroimidazo[1,2-a]pyrimidine, and the air in the flask was replaced with nitrogen. To this mixture was added 19 mL of dehydrated toluene, the mixture in the flask was degassed under reduced pressure, and then the air in the flask was replaced with nitrogen. This mixture was stirred for 8 hours while heated at 90° C., and then the mixture was cooled down to room temperature. After the reaction was completed, this reaction mixture was suction filtrated to give a solid. The obtained solid was washed with methanol and chloroform, whereby an insoluble material was removed. The obtained filtrate was concentrated under reduced pressure. The obtained solid was recrystallized with ethyl acetate and methanol to give 1.2 g of a gray solid in a 39% yield. The synthesis scheme of 2,7tip2SF is shown in Formula (b-1) below.

### [Chemical Formula 18]

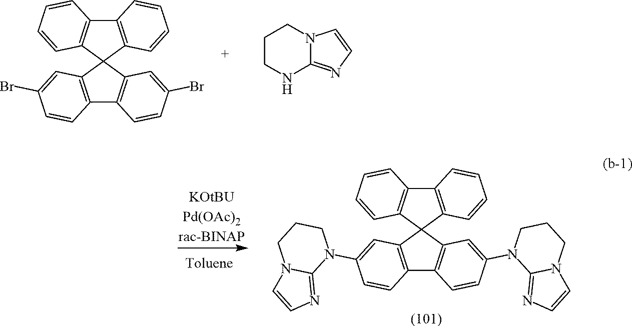

By a train sublimation method, 1.2 g of the obtained gray solid was purified by heating at 285° C. for 17 hours under a pressure of 3.1 Pa with an argon flow rate of 5 mL/min. As a result, 0.33 g of the target white solid was obtained at a 28% collection rate.

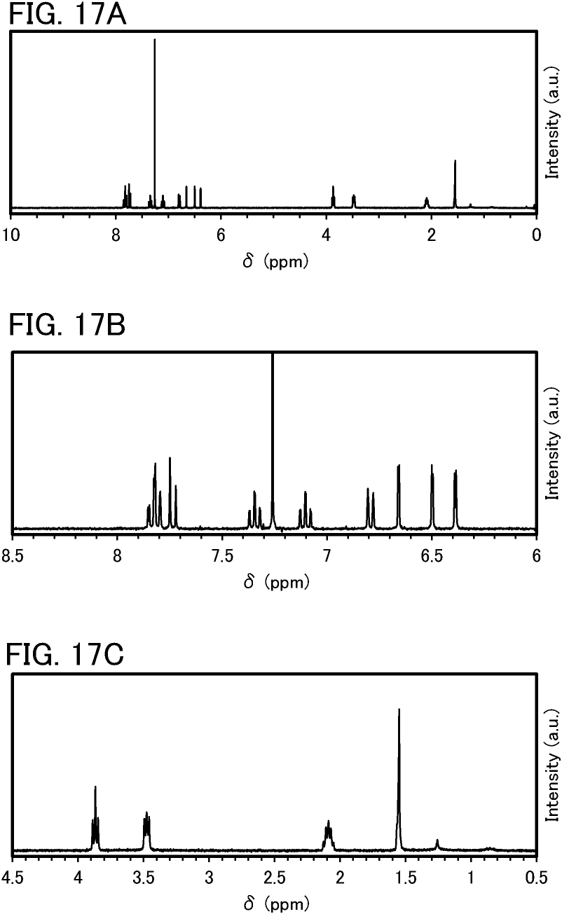

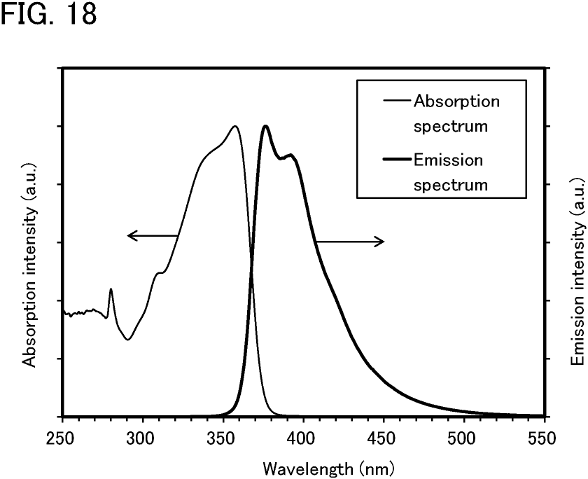

FIGS. 17A to 17C show a ¹H-NMR spectrum of 2,6tip2Py after the purification by sublimation. Results of ¹H NMR measurements are shown below. The results reveal that 2,7tip2SF was obtained.

¹H NMR (CDCl₃, 300 MHz): δ = 7.84-7.81 (m, 4H), 7.73 (d, J = 7.9 Hz, 2H), 7.34 (td, J = 7.5 Hz, 1.1 Hz, 2H), 7.10 (td, J = 7.5 Hz, 1.1 Hz, 2H), 6.79 (d, J = 7.9 Hz, 2H), 6.66 (d, J = 2.0 Hz, 2H), 6.50 (d, J = 1.5 Hz, 2H), 6.39 (d, J = 2.0 Hz, 2H), 3.87 (t, J = 6.0 Hz, 4H), 3.48 (t, J = 5.7 Hz, 4H), 2.13-2.05 (m, 4H).

### <Measurement of Emission and Absorption Spectra>

An ultraviolet-visible absorption spectrum (hereinafter, simply referred to as an absorption spectrum) of 2,7tip2SF in a toluene solution and an emission spectrum thereof were measured. The absorption spectrum was measured with an ultraviolet-visible spectrophotometer (V-770, produced by JASCO Corporation). The emission spectrum was measured with a fluorescence spectrophotometer (FP-8600, manufactured by JASCO Corporation). FIG. 18 shows the obtained absorption and emission spectra of 2,7tip2SF in the toluene solution. The horizontal axis represents the wavelength and the vertical axes represent the absorption intensity and the emission intensity.

As shown in FIG. 18, in the case of 2,7tip2SF in the toluene solution, an absorption peak was observed at around 359 nm, and an emission peak was observed at around 376 nm.

### Example 3

In this example, the organic compounds synthesized in Examples 1 and 2 are used to explain the solubility of the organic compound of one embodiment of the present invention. The solubility tests of this example were performed at room temperature (RT) at one atmosphere.

### <Solubility Test of 2,6tip2Py by Visible Inspection>

Into a 110 mL sample bottle, 1.21 mg of 2,6tip2Py was put and 10 mL of water was added thereto. This mixture was irradiated with ultrasonic waves for one minute. A precipitated white powder was found by visual inspection for an insoluble residue. After further addition of 10 mL of water and one-minute ultrasonic wave irradiation, a precipitated white powder was found by visual inspection. This procedure was repeated until the dissolution was confirmed by visual inspection.

The precipitated white powder was found until the total amount of water reached 100 mL. From the results, the measurement in the solubility test by visual inspection was found to be unattainable.

### <Solubility Test of 2,7tip2SF by Visible Inspection>

Into a 110 mL sample bottle, 1.23 mg of 2,7tip2SF was put and 10 mL of water was added thereto. This mixture was irradiated with ultrasonic waves for one minute. A precipitated white powder was found by visual inspection for an insoluble residue. After further addition of 10 mL of water and one-minute ultrasonic wave irradiation, a precipitated pale yellow powder was found by visual inspection. This procedure was repeated until the dissolution was confirmed by visual inspection.

The precipitated pale yellow powder was found until the total amount of water reached 100 mL. From the results, the measurement in the solubility test by visual inspection was found to be unattainable.

### (Reference Example 1)

### <Solubility Test of hpp2Py by Visual Inspection>

Into a 5 mL sample bottle, 50.2 mg of 1,1′-pyridine-2,6-diyl-bis(1,3,4,6,7,8-hexahydro-2H-pyrimido[1,2-a]pyrimidine) (abbreviation: hpp2Py) having a structure where a hydropyrimidine ring is substituted for the imidazole ring of the guanidine skeleton in 2,6tip2Py, which is an organic compound of one embodiment of the present invention, was put, and 1.0 mL of water was added thereto. The dissolution was found by visual inspection for an insoluble residue.

The results indicate that the weight of hpp2Py dissolved in 1.0 mL of water is greater than or equal to 50.2 mg. The weight fraction of the solubility of hpp2Py in water is higher than or equal to 4.8 × 10⁻².

### (Reference Example 2)

### <Solubility Test of 2,7hpp2SF by Visual Inspection>

Into a 20 mL sample bottle, 1.16 mg of 1,1′-(9,9′-spirobi[9H-fluorene]-2,7-diyl)bis(1,3,4,6,7,8-hexahydro-2H-pyrimido[1,2-a]pyrimidine) (abbreviation: 2,7hpp2SF) having a structure where a hydropyrimidine ring is substituted for the imidazole ring of the guanidine skeleton in 2,7tip2SF, which is an organic compound of one embodiment of the present invention, was put, and 0.5 mL of water was added thereto. This mixture was irradiated with ultrasonic waves for one minute. A precipitated white powder was found by visual inspection for an insoluble residue. After further addition of 0.5 mL of water and one-minute ultrasonic wave irradiation, a precipitated white powder was found by visual inspection. This procedure was repeated until the dissolution was confirmed by visual inspection.

The precipitated white powder was found until the total amount of water reached 3.0 mL. After further addition of 0.5 mL of water and ultrasonic wave irradiation, no precipitated white powder was found.

The results indicate that the weight of 2,7hpp2SF which can be dissolved in 1.0 mL of water is greater than or equal to 0.33 mg and less than 0.39 mg. The weight fraction of the solubility of 2,7hpp2SF in water is higher than or equal to 3.3 × 10⁻⁴ and lower than 3.9 × 10⁻⁴.

The solubility in water of each of 2,6tip2Py and 2,7tip2SF, which failed to be calculated by the above experimental method, was calculated by liquid chromatography mass spectrometry (LC/MS).

### <Solubility Test of 2,6tip2Py by LC/MS>

In the LC-MS analysis, liquid chromatography (LC) separation was carried out with ACQUITY UPLC manufactured by Waters Corporation, and MS analysis (mass spectrometry) was carried out with Xevo G2 Tof MS manufactured by Waters Corporation. Acquity UPLC BEH C8 (2.1 × 100 mm, 1.7 µm) was used as a column for the LC separation. Acetonitrile was used for Mobile Phase A and a 0.1 % aqueous solution of formic acid was used for Mobile Phase B. The amount of injection of the sample was 5.0 µL. Note that in the analysis, the wavelength of a photodiode array detector was set to 270 nm.

Into a 5 mL sample bottle, 1 mg of 2,6tip2Py was put and 1 mL of chloroform was added thereto. This mixture was irradiated with ultrasonic waves for five minutes. After it was confirmed that the solid was completely dissolved, this solution was diluted by five times with acetonitrile, whereby the concentration of the solution was adjusted to 200 mg/L. This solution was diluted with acetonitrile, whereby a solution with a concentration of 40 mg/L, a solution with a concentration of 20 mg/L, and a solution with a concentration of 10 mg/L were prepared. The prepared solutions were subjected to LC/MS, and the peak area values derived from 2,6tip2Py, which were obtained at the respective solution concentrations, were used to form calibration curves.

Next, the solubility of 2,6tip2Py in water was measured.

Into a 5 mL sample bottle, 1 mg of 2,6tip2Py was put and 1 mL of water was added thereto. This mixture was irradiated with ultrasonic waves for five minutes. This mixture was filtered through a membrane filter to remove the solid, and the resulting filtrate was diluted by five times with acetonitrile. The obtained solution was subjected to LC/MS.

From the calibration curve and the signal intensity obtained by LC/MS, it is found that the solubility of 2,6tip2Py in 1 mL of water is 0.018 mg. The weight fraction of the solubility of 2,6tip2Py in water is higher than or equal to 1.8 × 10⁻⁵.

### <Solubility Test of 2,7tip2SF by LC/MS>

Into a 5 mL sample bottle, 1 mg of 2,7tip2SF was put and 1 mL of water was added thereto. This mixture was irradiated with ultrasonic waves for five minutes. This mixture was filtered through a membrane filter to remove the solid, and the resulting filtrate was diluted by five times with acetonitrile. The obtained solution was subjected to LC/MS.

However, the peak area value derived from 2,7tip2SF failed to be obtained through the LC/MS. This means that 2,7tip2SF is an organic compound that is insoluble in water.

The above-described results reveal that 2,6tip2Py and 2,7tip2SF are organic compounds with very low solubility in water. By contrast, hpp2Py and 2,7hpp2SF, each having a structure where a hydropyrimidine ring is substituted for the imidazole ring of the guanidine skeleton in the corresponding aforementioned organic compound, have high solubility in water as described in the reference examples. Hence, it is found that the structure where the guanidine skeleton includes an imidazole ring is effective in reducing the low solubility in water.

It is thus found that the organic compound of one embodiment of the present invention represented by General Formula (G1) can be favorably used for a light-emitting device whose fabrication process includes processing using water or a chemical solution containing water as a solvent (i.e., a light-emitting device involving processing by a lithography method).

### Example 4

In this example, Light-emitting device 1 and Light-emitting device 2, which respectively use the organic compounds synthesized in Example 1 and Example 2, are described. Structural formulae of the organic compounds used for Light-emitting device 1 and 2 are shown below.

### [Chemical Formula 19]

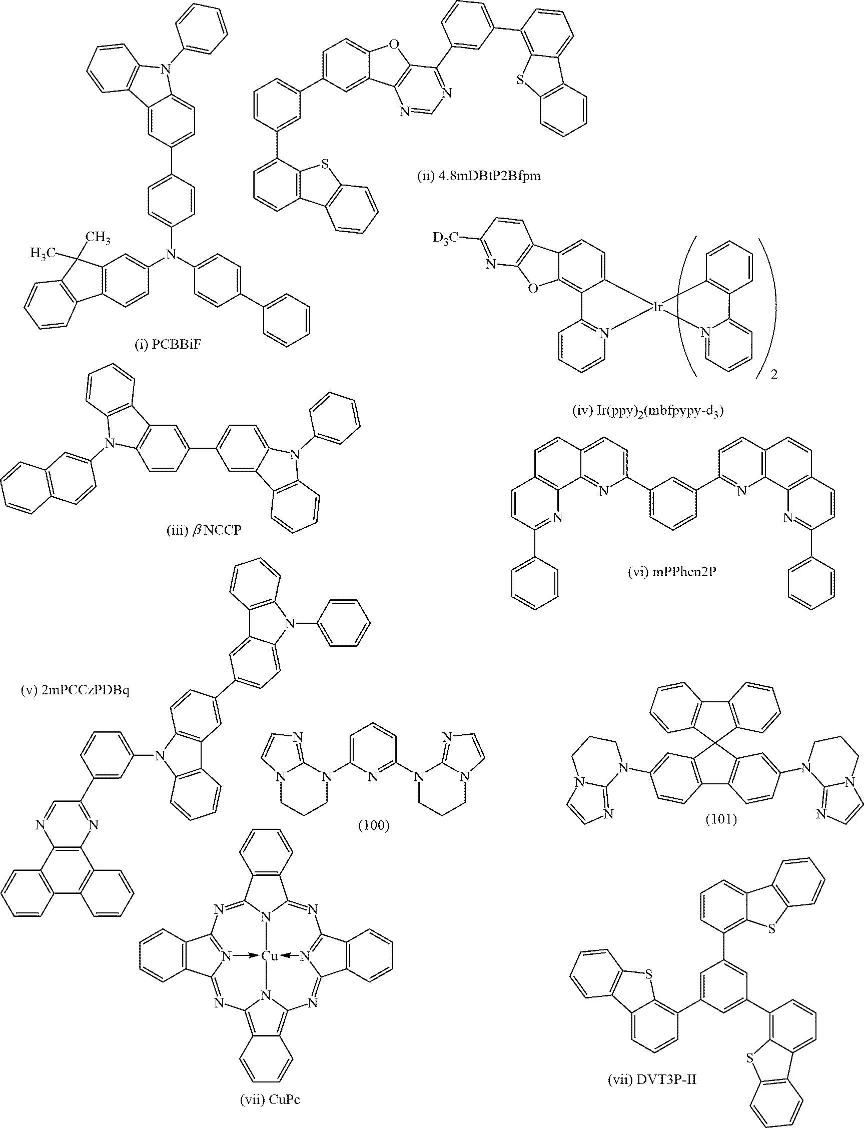

### (Method of Fabricating Light-Emitting Device 1)

First, as a reflective electrode, an alloy containing silver (Ag), palladium (Pd), and copper (Cu), i.e., an Ag-Pd-Cu (APC) film, was deposited over a glass substrate to a thickness of 100 nm by a sputtering method, and then, as a transparent electrode, indium tin oxide containing silicon oxide (ITSO) was deposited to a thickness of 100 nm by a sputtering method, whereby the first electrode 101 was formed. The electrode area was set to 4 mm² (2 mm × 2 mm). Note that the transparent electrode functions as the anode, and the transparent electrode and the reflective electrode can be collectively regarded as the first electrode 101.

Next, in pretreatment for forming the light-emitting device over a substrate, the surface of the substrate was washed with water, baking was performed at 200° C. for one hour, and then UV ozone treatment was performed for 370 seconds.

After that, the substrate was transferred into a vacuum evaporation apparatus where the pressure was reduced to approximately 10⁻⁴ Pa, and was subjected to heat treatment at 170° C. for 30 minutes in a heating chamber of the vacuum evaporation apparatus, and then the substrate was cooled down for approximately 30 minutes.

Then, the substrate provided with the first electrode was fixed to a substrate holder provided in the vacuum evaporation apparatus such that the surface on which the first electrode was formed faced downward. Over the first electrode, N-(biphenyl-4-yl)-N-[4-(9-phenyl-9H-carbazol-3-yl)phenyl]-9,9-dimethyl-9H-fluoren-2-amine (abbreviation: PCBBiF) (Structural Formula (i)) and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby the hole-injection layer was formed.

Over the hole-injection layer, PCBBiF was deposited by evaporation to a thickness of 60 nm, whereby a first hole-transport layer was formed.

Then, over the first hole-transport layer, 4,8-bis[3-(dibenzothiophen-4-yl)phenyl]-[1]benzofuro[3,2-d]pyrimidine (abbreviation: 4,8mDBtP2Bfpm) (Structural Formula (ii)), 9-(2-naphthyl)-9′-phenyl-9H,9′H-3,3′-bicarbazole (abbreviation: βNCCP) (Structural Formula (iii)), and [2-d3-methyl-(2-pyridinyl-ĸN)benzofuro[2,3-b]pyridine-κC]bis[2-(2-pyridinyl-κN)phenyl-κC]iridium(III) (abbreviation: Ir(ppy)₂(mbfpypy-d₃)) (Structural Formula (iv)) were deposited by co-evaporation to a thickness of 40 nm such that the weight ratio of 4,8mDBtP2Bfpm to βNCCP and Ir(ppy)₂(mbfpypy-d₃) was 0.5:0.5:0.1, whereby a first light-emitting layer was formed.

Next, 2-{3-[3-(N-phenyl-9H-carbazol-3-yl)-9H-carbazol-9-yl]phenyl}dibenzo[f,h]quinoxaline (abbreviation: 2mPCCzPDBq) (Structural Formula (v)) was deposited by evaporation to a thickness of 10 nm, and then 2,2′-(1,3-phenylene)bis(9-phenyl-1,10-phenanthroline) (abbreviation: mPPhen2P) (Structural Formula (vi)) was deposited by evaporation to a thickness of 15 nm to form a first electron-transport layer.

After the first electron-transport layer was formed, mPPhen2P and 2,6tip2Py represented by Structural Formula (100) above, which is an organic compound of one embodiment of the present invention, were deposited by co-evaporation to a thickness of 5 nm such that the weight ratio of mPPhen2P to 2,6tip2Py was 1:1, whereby a first layer was formed. Then, copper phthalocyanine (abbreviation: CuPc) (Structural Formula (vii)) was deposited to a thickness of 2 nm, whereby a third layer for smooth transfer of electrons between the first layer and a second layer was formed. Furthermore, PCBBiF and OCHD-003 were deposited by co-evaporation to a thickness of 10 nm such that the weight ratio of PCBBiF to OCHD-003 was 1:0.15, whereby an intermediate layer including the first to third layers was formed.

Over the intermediate layer, PCBBiF was then deposited by evaporation to a thickness of 40 nm, whereby a second hole-transport layer was formed.

Over the second hole-transport layer, 4,8mDBtP2Bfpm, βNCCP, and Ir(ppy)₂(mbfpypy-d₃) were deposited by co-evaporation to a thickness of 40 nm such that the weight ratio of 4,8mDBtP2Bfpm to βNCCP and Ir(ppy)₂(mbfpypy-d₃) was 0.5:0.5:0.1, whereby a second light-emitting layer was formed.

Then, 2mPCCzPDBq was deposited by evaporation to a thickness of 20 nm, and mPPhen2P was further deposited by evaporation to a thickness of 20 nm, whereby a second electron-transport layer was formed.

Over the second electron-transport layer, lithium fluoride (LiF) and ytterbium (Yb) were deposited by co-evaporation to a thickness of 1.5 nm such that the volume ratio of LiF to Yb was 2:1 to form the electron-injection layer, and lastly silver (Ag) and magnesium (Mg) were deposited by co-evaporation to a thickness of 15 nm such that the volume ratio of Ag to Mg was 1:0.1 to form the second electrode, whereby Light-emitting device 1 was fabricated.

The second electrode is a transflective electrode having a function of reflecting light and a function of transmitting light; thus, the light-emitting device of this example is a top-emission tandem device in which light is extracted through the second electrode. Over the second electrode, 4,4′,4″-(benzene-1,3,5-triyl)tri(dibenzothiophene) (abbreviation: DBT3P-II) (Structural Formula (viii)) was deposited by evaporation to a thickness of 70 nm as a cap layer to improve light extraction efficiency.

### (Method of Fabricating Light-Emitting Device 2)

Light-emitting device 2 is different from Light-emitting device 1 in that 2,7tip2SF represented by Structural Formula (101) above, which is an organic compound of one embodiment of the present invention, is substituted for 2,6tip2Py used for the first layer of the intermediate layer of Light-emitting device 1. In other words, Light-emitting device 2 was fabricated in the same manner as Light-emitting device 1 except that mPPhen2P and 2,7tip2SF were deposited by co-evaporation to a thickness of 5 nm such that the weight ratio of mPPhen2P to 2,7tip2SF was 1:1 to form the first layer.

The device structures of Light-emitting devices 1 and 2 are listed in the following table.

TABLE 1

| | | Thickness | Light-emitting device 1 | Light-emitting device 2 |
|---|---|---|---|---|
| Cap layer | | 70 nm | DBT3P-II | |
| Second electrode | | 15 nm | AgMg (1:0.1) | |
| Electron-injection layer | | 1.5 nm | LiF:Yb (2:1) | |
| Second electron-transport layer | 2 | 20 nm | mPPhen2P | |
| | 1 | 20 nm | 2mPCCzPDBq | |
| Second light-emitting layer | | 40 nm | 4,8mDBtP2Bfpm:βNCCP:Ir(ppy)₂(nbfpypy-d₃) (0.5:0.5:0.1) | |
| Second hole-transport layer | | 40 nm | PCBBiF | |
| Intermediate layer | Second layer | 10 nm | PCBBiF:OCHD-003 (1:0.15) | |
| | Third layer | 2 nm | CuPc | |
| | First layer | 5 nm | mPPhen2P:2,6tip2Py (1:1) | mPPhen2P:2,7tip2SF (1:1) |
| First electron-transport layer | | 15 nm | mPPhen2P | |
| | | 10 nm | 2mPCCzPDBq | |
| First light-emitting layer | | 40 nm | 4,8mDBtP2Bfpnt:βNCCP:Ir(ppy)₂(mbfpypy-d₃) (0.5:0.5:0.1) | |
| First hole-transport layer | | 60 nm | PCBBiF | |
| Hole-injection layer | | 10 nm | PCBBiF:OCHD-003 (1:0.03) | |
| First electrode | 2 | 100 nm | ITSO | |
| | 1 | 100 nm | APC | |

Light-emitting devices 1 and 2 were sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. Then, the initial characteristics of the light-emitting devices were measured.

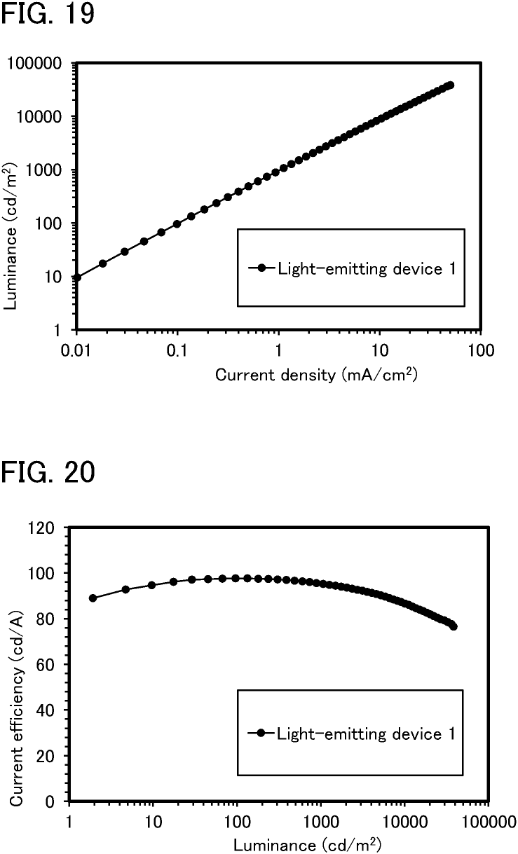

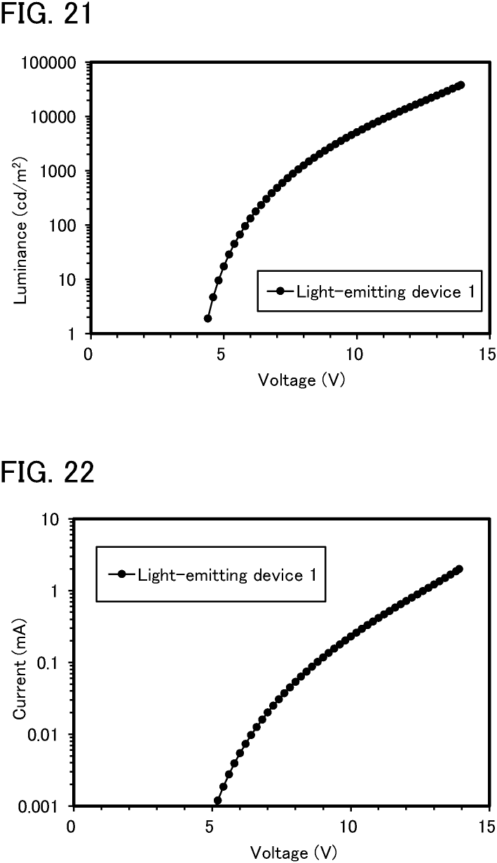

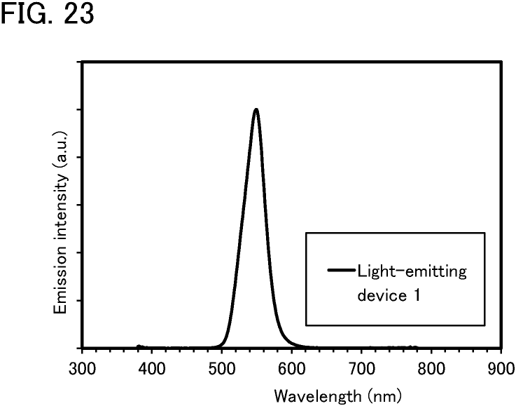

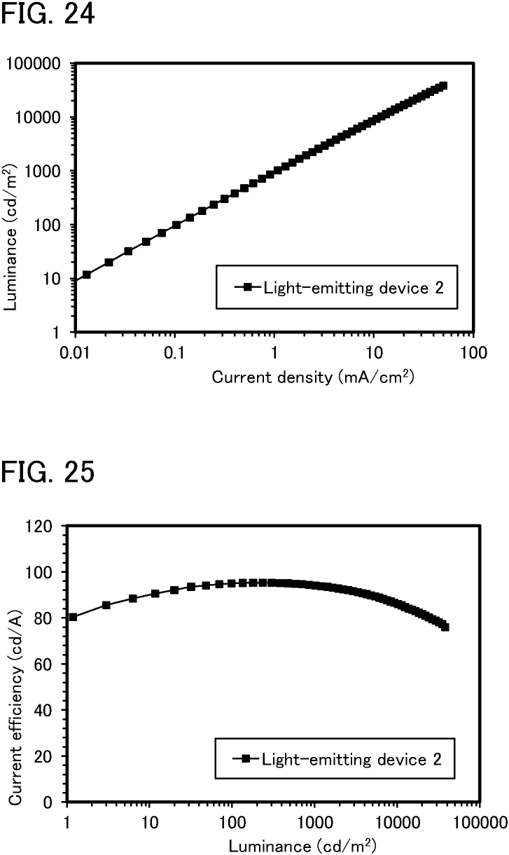

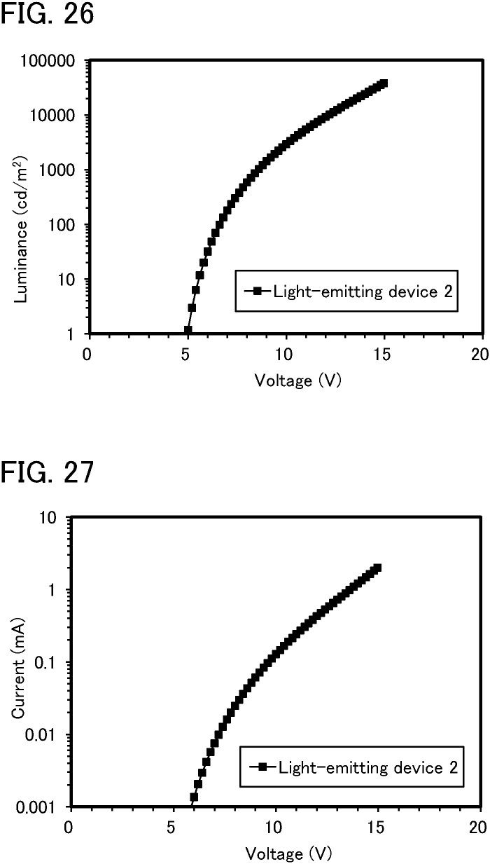

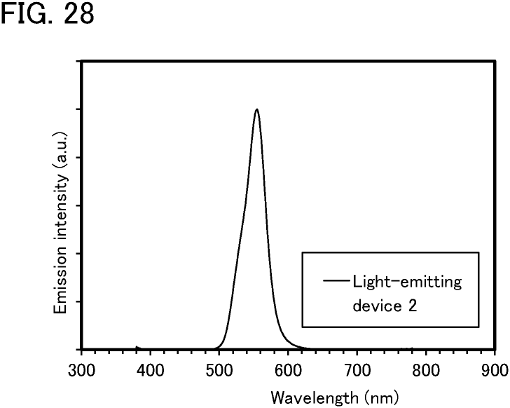

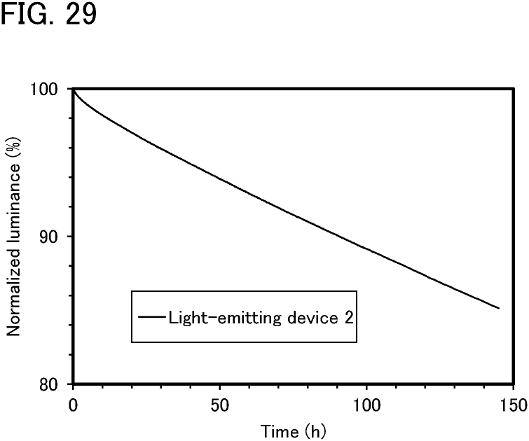

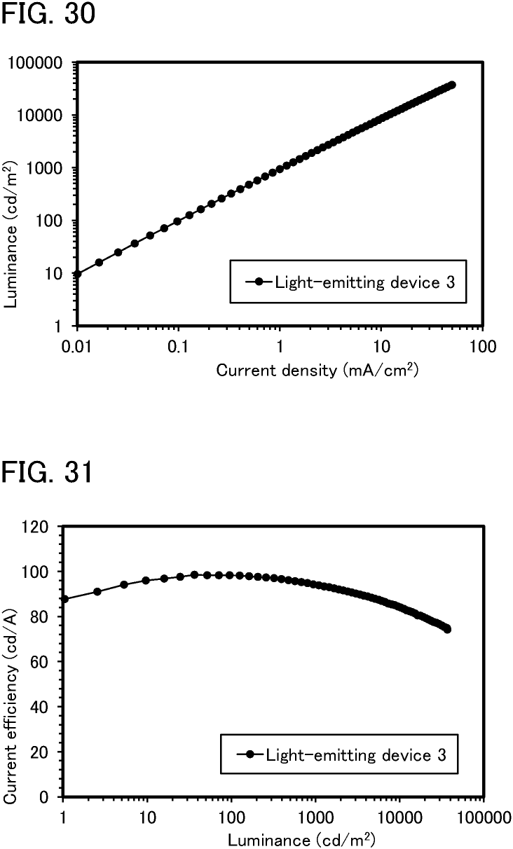

FIG. 19 shows the luminance-current density characteristics of Light-emitting device 1. FIG. 20 shows the current efficiency-luminance characteristics thereof. FIG. 21 shows the luminance-voltage characteristics thereof. FIG. 22 shows the current-voltage characteristics thereof. FIG. 23 shows the electroluminescence spectrum thereof. FIG. 24 shows the luminance-current density characteristics of Light-emitting device 2. FIG. 25 shows the current efficiency-luminance characteristics thereof. FIG. 26 shows the luminance-voltage characteristics thereof. FIG. 27 shows the current-voltage characteristics thereof. FIG. 28 shows the electroluminescence spectrum thereof. FIG. 29 shows a luminance change over driving time when Light-emitting device 2 was driven at a constant current of 2 mA (50 mA/cm²). The following table shows the main characteristics at a luminance of approximately 1000 cd/m². The luminance, CIE chromaticity, and electroluminescence spectra were measured at normal temperature with a spectroradiometer (SR-UL1R manufactured by TOPCON TECHNOHOUSE CORPORATION).

TABLE 2

| | Voltage (V) | Current (mA) | Current density (mA/cm²) | Chromaticity x | Chromaticity y | Luminance (cd/m²) | Current efficiency (cd/A) |
|---|---|---|---|---|---|---|---|
| Light-emitting device 1 | 7.8 | 0.045 | 1.1 | 0.303 | 0.679 | 1066 | 95.2 |
| Light-emitting device 2 | 8.6 | 0.043 | 1.1 | 0.332 | 0.653 | 1022 | 94.1 |

FIG. 19, FIG. 20, FIG. 21, FIG. 22, FIG. 23, FIG. 24, FIG. 25, FIG. 26, FIG. 27, FIG. 28, and FIG. 29 and the above table reveal that Light-emitting devices 1 and 2 have favorable light-emitting characteristics.

### Example 5

In this example, Light-emitting device 3 and Light-emitting device 4, which respectively use the organic compounds synthesized in Example 1 and Example 2, are described. The structural formulae of the organic compounds used for Light-emitting devices 3 and 4 are not shown here because the organic compounds are the same as those used for Light-emitting devices 1 and 2.

### (Method of Fabricating Light-Emitting Device 3)

Light-emitting device 3 is different from Light-emitting device 1 in that the weight ratio of mPPhen2P to 2,6tip2Py used for the first layer of the intermediate layer of Light-emitting device 1 was 1:0.5 and that processing by a photolithography method and heat treatment were performed after the formation of the second electron-transport layer. The other layers were fabricated in a manner similar to that for Light-emitting device 1.

The processing by a photolithography method and the heat treatment are described. The substrate was taken out from the vacuum evaporation apparatus and exposed to the air, and then aluminum oxide was deposited to a thickness of 30 nm by an ALD method using trimethylaluminum (abbreviation: TMA) as a precursor and water vapor as an oxidizer to form a first sacrificial layer.

Over the first sacrificial layer, a composite oxide containing indium, gallium, zinc, and oxygen (abbreviation: IGZO) was deposited to a thickness of 50 nm by a sputtering method to form a second sacrificial layer.

A resist was formed using a photoresist over the second sacrificial layer, and processing was performed by a photolithography method to form a slit having a width of 3 µm in a position 3.5 µm away from an end portion of the first electrode.

Specifically, the second sacrificial layer was processed using a chemical solution containing a phosphoric acid solution with the use of a resist as a mask, and then the first sacrificial layer was processed using an etching gas containing fluoroform (CHF₃) and helium (He) at a flow rate ratio of CHF₃:He = 1:9. After that, the second electron-transport layer, the second light-emitting layer, the second hole-transport layer, the intermediate layer, the first electron-transport layer, the first light-emitting layer, the first hole-transport layer, and the hole-injection layer were processed using an etching gas containing oxygen (O₂).

After the processing by a photolithography method, the first and second sacrificial layers were removed using a basic chemical solution containing water as a solvent, so that the second electron-transport layer was exposed. Then, the substrate was transferred into a vacuum evaporation apparatus where the pressure was reduced to approximately 10⁻⁴ Pa, and heat treatment was performed at 110° C. for 1 hour in a heating chamber of the vacuum evaporation apparatus.

As described above, water or a chemical solution containing water as a solvent is used in the processing by a photolithography method and the heat treatment.

### (Method of Fabricating Light-Emitting Device 4)

Light-emitting device 4 is different from Light-emitting device 2 in that the weight ratio of mPPhen2P to 2,7tip2SF used for the first layer of the intermediate layer of Light-emitting device 2 was 1:0.5 and that processing by a photolithography method and heat treatment were performed after the formation of the second electron-transport layer. The other layers were fabricated in a manner similar to that for Light-emitting device 2. Note that the processing by a photolithography method and heat treatment were fabricated in a manner similar to that for Light-emitting device 3.

The device structures of Light-emitting devices 3 and 4 are listed in the following table.

TABLE 3

| | | Thickness | Light-emitting device 3 | Light-emitting device 4 |
|---|---|---|---|---|
| Cap layer | | 70 nm | DBT3P-II | |
| Second electrode | | 15 nm | AgMg (1:0.1) | |
| Electron-injection layer | | 1.5 nm | LiF:Yb (2:1) | |
| - | | - | Processing by photolithography and heat treatment were performed. | |
| Second electron-transport layer | 2 | 20 nm | mPPhen2P | |
| | 1 | 20 nm | 2mPCCzPDBq | |
| Second light-emitting layer | | 40 nm | 4,8mDBtP2Bfpm:βNCCP:Ir(ppy)₂(mbfpypy-d₃) (0.5:0.5:0.1) | |
| Second hole-transport layer | | 40 nm | PCBBiF | |
| Intermediate layer | Second layer | 10 nm | PCBBiF:OCHD-003 (1:0.15) | |
| | Third layer | 2 nm | CuPc | |
| | First layer | 5 nm | mPPhen2P:2,6tip2Py (1:0.5) | mPPhen2P:2,7tip2SF (1:0.5) |
| First electron-transport layer | | 15 nm | mPPhen2P | |
| | | 10 nm | 2mPCCzPDBq | |
| First light-emitting layer | | 40 nm | 4,8mDBtP2Bfpm:βNCCP:Ir(ppy)₂(mbfpypy-d₃) (0.5:0.5:0.1) | |
| First hole-transport layer | | 60 nm | PCBBiF | |
| Hole-injection layer | | 10 nm | PCBBiF:OCHD-003 (1:0.03) | |
| First electrode | 2 | 100 nm | ITSO | |
| | 1 | 100 nm | APC | |

Light-emitting devices 3 and 4 were sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. Then, the initial characteristics of the light-emitting devices were measured.

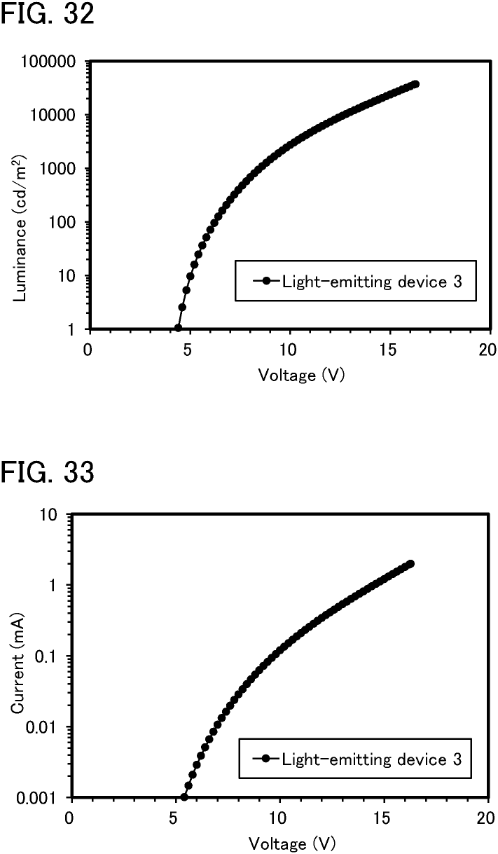

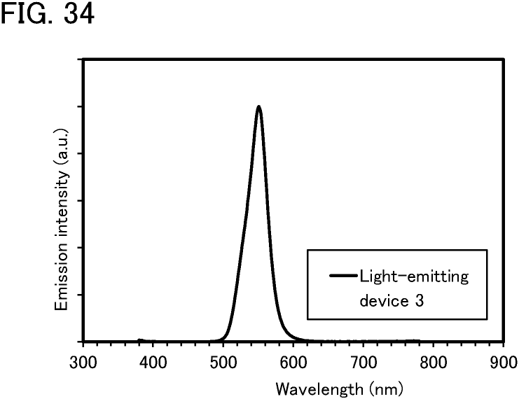

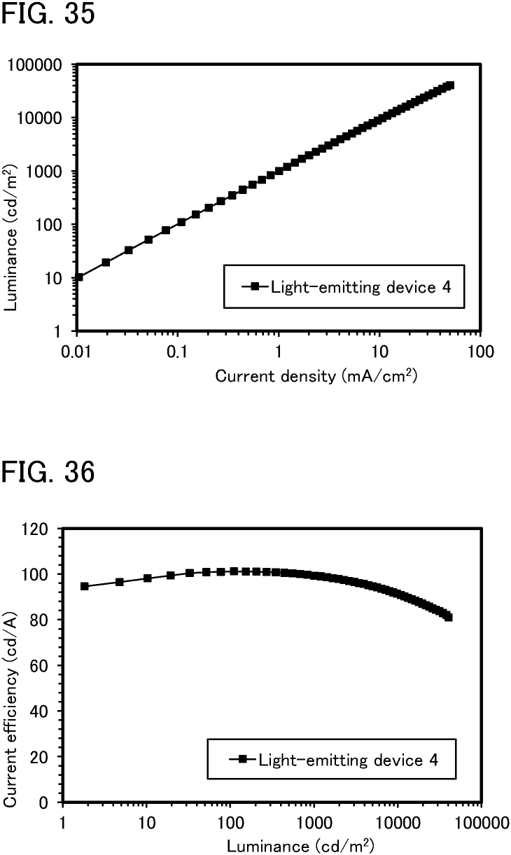

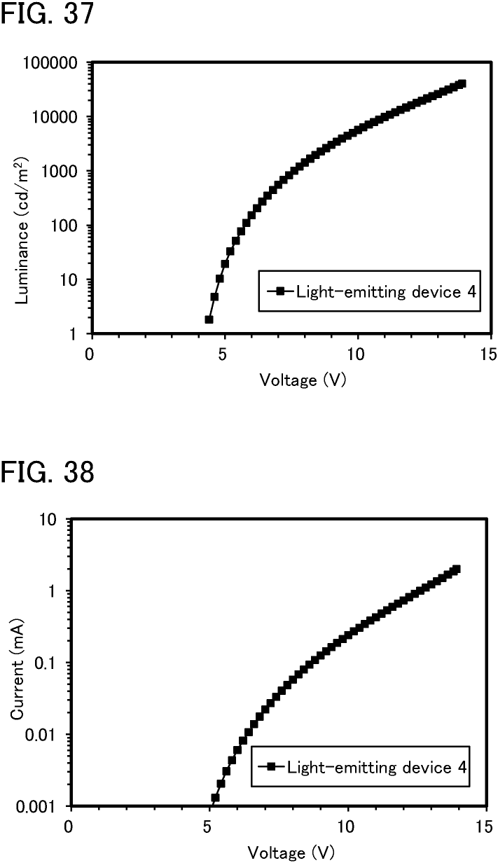

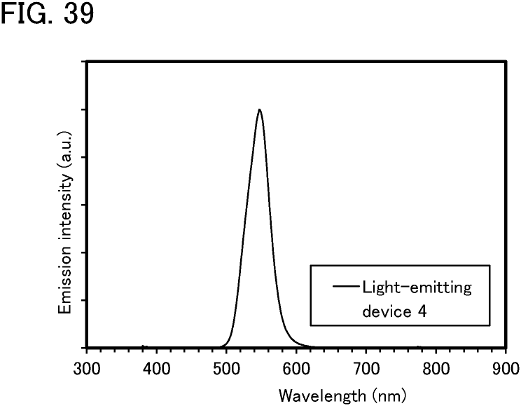

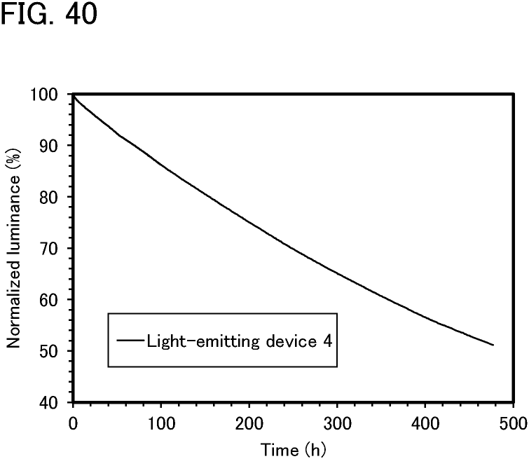

FIG. 30 shows the luminance-current density characteristics of Light-emitting device 3. FIG. 31 shows the current efficiency-luminance characteristics thereof. FIG. 32 shows the luminance-voltage characteristics thereof. FIG. 33 shows the current-voltage characteristics thereof. FIG. 34 shows the electroluminescence spectrum thereof. FIG. 35 shows the luminance-current density characteristics of Light-emitting device 4. FIG. 36 shows the current efficiency-luminance characteristics thereof. FIG. 37 shows the luminance-voltage characteristics thereof. FIG. 38 shows the current-voltage characteristics thereof. FIG. 39 shows the electroluminescence spectrum thereof. FIG. 40 shows a luminance change over driving time when Light-emitting device 4 was driven at a constant current of 2 mA (50 mA/cm²). The following table shows the main characteristics at a luminance of approximately 1000 cd/m². The luminance, CIE chromaticity, and electroluminescence spectra were measured at normal temperature with a spectroradiometer (SR-UL1R manufactured by TOPCON TECHNOHOUSE CORPORATION).

TABLE 4

| | Voltage (V) | Current (mA) | Current density (mA/cm²) | Chromaticity x | Chromaticity y | Luminance (cd/m²) | Current efficiency (cd/A) |
|---|---|---|---|---|---|---|---|
| Light-emitting device 3 | 8.4 | 0.040 | 1.0 | 0.309 | 0.674 | 940 | 94.2 |
| Light-emitting device 4 | 7.6 | 0.040 | 1.0 | 0.298 | 0.682 | 1005 | 99.3 |

FIG. 30, FIG. 31, FIG. 32, FIG. 33, FIG. 34, FIG. 35, FIG. 36, FIG. 37, FIG. 38, FIG. 39, and FIG. 40 and the above table reveal that Light-emitting devices 3 and 4 have favorable light-emitting characteristics. It is also found that the organic compound of one embodiment of the present invention has low solubility in water as described above and thus can be favorably used even when the fabrication process includes processing using water or a chemical solution containing water as a solvent (i.e., processing by a lithography method).

### Example 6

In this example, a synthesis method of 8-(9,9′-spirobi[9H-fluoren]-2-yl)-5,6,7,8-tetrahydroimidazo[1,2-a]pyrimidine (abbreviation: tipSF) represented by Structural Formula (113) in Embodiment 1 is described. The structure of tipSF is shown below.

### [Chemical Formula 20]

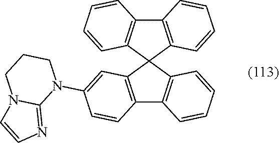 [SMILES: c1ccc2c(c1)-c1ccccc1C21c2ccccc2-c2ccc(N3CCCn4ccnc43)cc21]

### <Synthesis of TipSF>

Into a 300 mL three-neck flask were put 18 g (46 mmol) of 2-bromo-9,9′-spirobi[9H-fluorene], 13 g (0.12 mol) of potassium-tert-butoxide (abbreviation: KOtBu), 1.7 g (2.7 mmol) of (±)-2,2′-bis(diphenylphosphino)-1,1′-binaphthyl (abbreviation: rac-BINAP), and 7.0 g (57 mmol) of 5,6,7,8-tetrahydroimidazo[1,2-a]pyrimidine, and the air in the flask was replaced with nitrogen. To the mixture, 150 mL of dehydrated toluene was added, and this mixture was degassed by being stirred under reduced pressure. To this mixture was added 0.41 g (1.8 mmol) of palladium acetate (abbreviation: Pd(OAc)₂), and the mixture was stirred under a nitrogen stream at 90° C. for 8 hours. Then, the mixture was cooled down to room temperature. An insoluble material was removed by suction filtration, and the filtrate was subjected to extraction with toluene. After that, the extracted solution was concentrated to give a residue. A small amount of toluene was added to the obtained residue, followed by ultrasonic wave irradiation. A solid was collected by suction filtration to give 11 g of the target pale yellow solid at a 55% yield. The synthesis scheme of tipSF is shown in (c-1) below.

### [Chemical Formula 21]

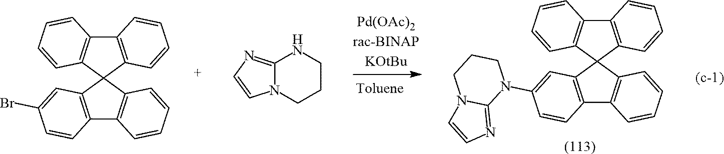

By a train sublimation method, 11 g of the obtained pale yellow solid was purified by heating at 225° C. for 48 hours under a pressure of 6.0 Pa with an argon flow rate of 10 mL/min. As a result, 6.7 g of the target white solid was obtained at a 61% collection rate.

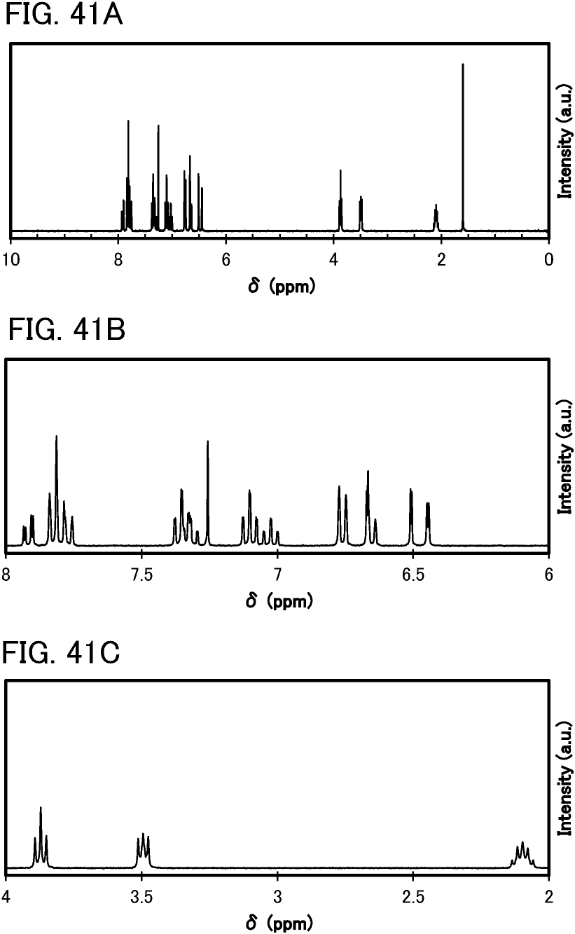

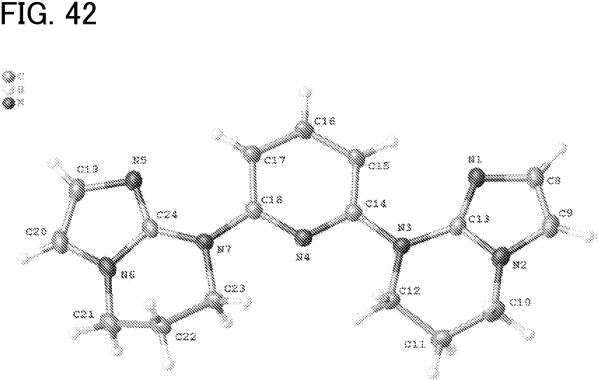

FIGS. 41A to 41C show a ¹H-NMR spectrum of tipSF after the purification by sublimation. Results of ¹H NMR measurements are shown below. The results reveal that tipSF was obtained.

¹H NMR (CDCl₃, 300 MHz): δ = 7.92 (dd, J = 8.4, J = 2.4 Hz, 1H), 7.84-7.76 (m, 4H), 7.38-7.29 (m, 3H), 7.10 (td, J = 7.5, J = 0.9 Hz, 2H), 7.02 (td, J = 7.5, J = 0.6 Hz, 1H), 6.76 (d, J = 7.2 Hz, 2H), 6.67-6.64 (m, 2H), 6.51 (sd, J = 1.5 Hz, 1H), 6.45 (sd, J = 1.8 Hz, 1H), 3.87 (t, J = 6.15 Hz, 2H), 3.50 (t, J = 5.7 Hz, 2H), 2.14-2.06 (m, 2H).

### Example 7

This example shows results of X-ray crystallography of the organic compound of one embodiment of the present invention. The analysis was to determine which nitrogen atom in a partial structure derived from 5,6,7,8-tetrahydroimidazo[1,2-a]pyrimidine was bonded to pyridine in a product of a coupling reaction with an aryl halide. Specifically, 2,6tip2Py, whose synthesis method is shown in Example 1, was subjected to X-ray crystallography with a single crystal X-ray crystallography system (XtaLAB Synergy-Custom, manufactured by Rigaku Corporation).

First, a white solid of 2,6tip2Py was recrystallized with hexane and ethyl acetate, whereby white prismatic crystals were obtained. Results of the X-ray crystallography of the obtained white prismatic crystals are illustrated in a molecular structure image in FIG. 42. FIG. 42 shows that the nitrogen atom in the 5-position of 5,6,7,8-tetrahydroimidazo[1,2-a]pyrimidine is bonded to pyridine in 2,6tip2Py.

From the above-described X-ray crystallography results, it is found that the peaks at 6.84 ppm and 6.65 ppm of the ¹H NMR spectrum of 2,6tip2Py (shown in FIGS. 15A to 15C) are peaks of the hydrogen atoms bonded to the carbon atoms at the 2- and 3-positions of 5,6,7,8-tetrahydroimidazo[1,2-a] pyrimidine.

Also in the ¹H NMR spectrum of 2,7tip2SF (shown in FIGS. 17A to 17C), whose synthesis method is shown in Example 2, peaks are observed at around 6.5 ppm, and probably these are also the peaks of the hydrogen atoms bonded to the carbon atoms at the 2- and 3-positions of 5,6,7,8-tetrahydroimidazo[1,2-a]pyrimidine. Hence, it is said that also in 2,7tip2SF, the nitrogen atom in the 5-position of 5,6,7,8-tetrahydroimidazo[1,2-a]pyrimidine is bonded to 9,9′-spirobi[9H-fluorene].

This application is based on Japanese Patent Application Serial No. 2022-075591 filed with Japan Patent Office on Apr. 29, 2022 and Japanese Patent Application Serial No. 2022-195343 filed with Japan Patent Office on Dec. 07, 2022, the entire contents of which are hereby incorporated by reference.

What is claimed is:

1. An organic compound represented by General Formula (G1): 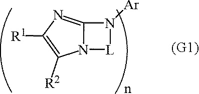 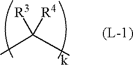
wherein, in General Formula (G1):
Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring;
each of R¹ and R² independently represents hydrogen, an alkyl group having 1 to 6 carbon atoms, a substituted or unsubstituted amino group, a substituted or unsubstituted aryl group having 6 to 13 carbon atoms forming a ring, or a substituted or unsubstituted heteroaryl group having 2 to 13 carbon atoms forming a ring;
n represents an integer greater than or equal to 1 and less than or equal to 6; and
L is a group represented by General Formula (L-1), and
wherein, in General Formula (L-1):
each of R³ and R⁴ independently represents hydrogen or an alkyl group having 1 to 6 carbon atoms; and
k represents an integer greater than or equal to 1 and less than or equal to 5.

2. The organic compound according to claim 1,
wherein when k is greater than or equal to 2, R³s are the same as or different from each other and R⁴s are the same as or different from each other.

3. The organic compound according to claim 1,
wherein each of the aromatic hydrocarbon group having 6 to 30 carbon atoms forming a ring and the heteroaromatic hydrocarbon group having 2 to 30 carbon atoms forming a ring is a group comprising a structure in which n hydrogen atom(s) is/are removed from any one of rings in any of aromatic hydrocarbons and heteroaromatic hydrocarbons represented by Structural Formula (Ar-1), Structural Formula (Ar-2), Structural Formula (Ar-3), Structural Formula (Ar-4), Structural Formula (Ar-5), Structural Formula (Ar-6), Structural Formula (Ar-7), Structural Formula (Ar-8), Structural Formula (Ar-9), Structural Formula (Ar-10), Structural Formula (Ar-11), Structural Formula (Ar-12), Structural Formula (Ar-13), Structural Formula (Ar-14), Structural Formula (Ar-15), Structural Formula (Ar-16), Structural Formula (Ar-17), Structural Formula (Ar-18), Structural Formula (Ar-19), Structural Formula (Ar-20), Structural Formula (Ar-21), Structural Formula (Ar-22), Structural Formula (Ar-23), Structural Formula (Ar-24), Structural Formula (Ar-25), Structural Formula (Ar-26), and Structural Formula (Ar-27), 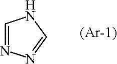 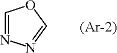 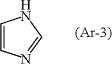 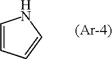 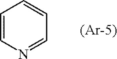 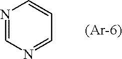 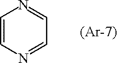 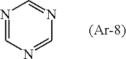 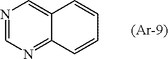 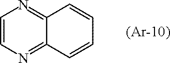 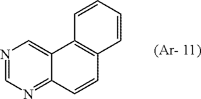 [SMILES: c1ccc2c(c1)ccc1ncncc12] 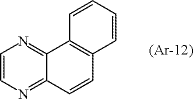 [SMILES: c1ccc2c(c1)ccc1nccnc12] 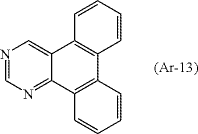 [SMILES: c1ccc2c(c1)c1ccccc1c1ncncc21] 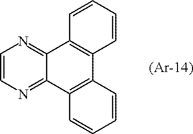 [SMILES: c1ccc2c(c1)c1ccccc1c1nccnc21] 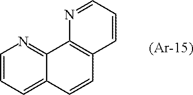 [SMILES: c1cnc2c(c1)ccc1cccnc12] 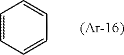 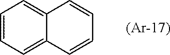 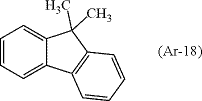 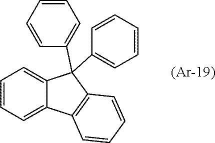 [SMILES: c1ccc(C2(c3ccccc3)c3ccccc3-c3ccccc32)cc1] 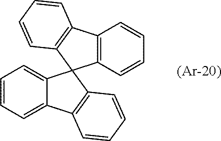 [SMILES: c1ccc2c(c1)-c1ccccc1C21c2ccccc2-c2ccccc21] 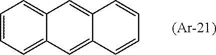 [SMILES: c1ccc2cc3ccccc3cc2c1] 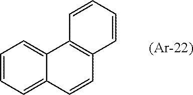 [SMILES: c1ccc2c(c1)ccc1ccccc12] 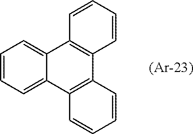 [SMILES: c1ccc2c(c1)c1ccccc1c1ccccc21] 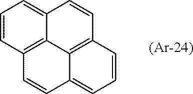 [SMILES: c1cc2ccc3cccc4ccc(c1)c2c34] 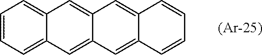 [SMILES: c1ccc2cc3cc4ccccc4cc3cc2c1] 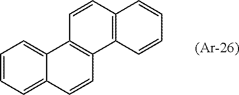 [SMILES: c1ccc2c(c1)ccc1c3ccccc3ccc21] 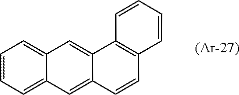 [SMILES: c1ccc2cc3c(ccc4ccccc43)cc2c1]
.

4. A light-emitting device comprising the organic compound according to claim 1.

5. A light-emitting apparatus comprising:
the light-emitting device according to claim 4; and
at least one of a transistor and a substrate.

6. An electronic device comprising:
the light-emitting apparatus according to claim 5; and
at least one of a sensor unit, an input unit, and a communication unit.

7. An organic compound represented by any one of General Formula (G2-1), General Formula (G2-2), and General Formula (G2-3): 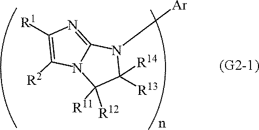 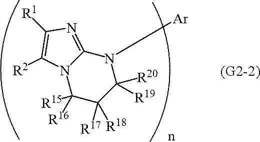 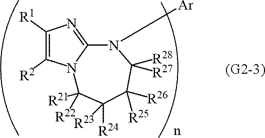
wherein, in General Formulae (G2-1) to (G2-3):
Ar represents a substituted or unsubstituted aromatic hydrocarbon group having 6 to 30 carbon atoms forming a ring or a substituted or unsubstituted heteroaromatic hydrocarbon group having 2 to 30 carbon atoms forming a ring;
each of R¹, R², and R¹¹ to R²⁸ independently represents hydrogen or an alkyl group having 1 to 6 carbon atoms; and
n represents an integer greater than or equal to 1 and less than or equal to 6.

8. The organic compound according to claim 7,
wherein each of the aromatic hydrocarbon group having 6 to 30 carbon atoms forming a ring and the heteroaromatic hydrocarbon group having 2 to 30 carbon atoms forming a ring is a group comprising a structure in which n hydrogen atom(s) is/are removed from any one of rings in any of aromatic hydrocarbons and heteroaromatic hydrocarbons represented by Structural Formula (Ar-1), Structural Formula (Ar-2), Structural Formula (Ar-3), Structural Formula (Ar-4), Structural Formula (Ar-5), Structural Formula (Ar-6), Structural Formula (Ar-7), Structural Formula (Ar-8), Structural Formula (Ar-9), Structural Formula (Ar-10), Structural Formula (Ar-11), Structural Formula (Ar-12), Structural Formula (Ar-13), Structural Formula (Ar-14), Structural Formula (Ar-15), Structural Formula (Ar-16), Structural Formula (Ar-17), Structural Formula (Ar-18), Structural Formula (Ar-19), Structural Formula (Ar-20), Structural Formula (Ar-21), Structural Formula (Ar-22), Structural Formula (Ar-23), Structural Formula (Ar-24), Structural Formula (Ar-25), Structural Formula (Ar-26), and Structural Formula (Ar-27), 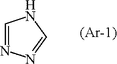 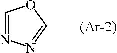 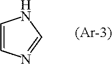 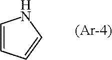 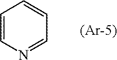 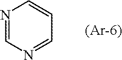 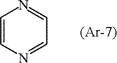 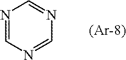 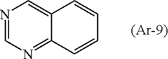 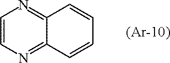 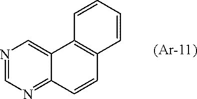 [SMILES: c1ccc2c(c1)ccc1ncncc12] 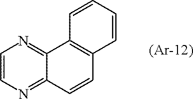 [SMILES: c1ccc2c(c1)ccc1nccnc12] 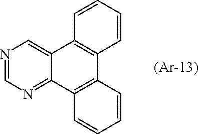 [SMILES: c1ccc2c(c1)c1ccccc1c1ncncc21] 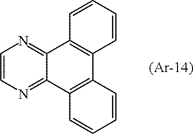 [SMILES: c1ccc2c(c1)c1ccccc1c1nccnc21] 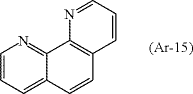 [SMILES: c1cnc2c(c1)ccc1cccnc12] 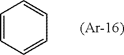 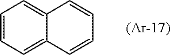 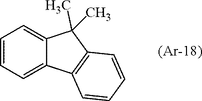 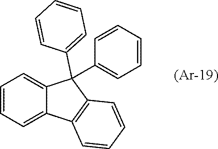 [SMILES: c1ccc(C2(c3ccccc3)c3ccccc3-c3ccccc32)cc1] 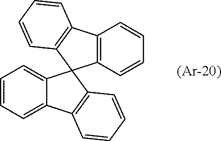 [SMILES: c1ccc2c(c1)-c1ccccc1C21c2ccccc2-c2ccccc21] 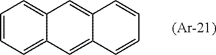 [SMILES: c1ccc2cc3ccccc3cc2c1] 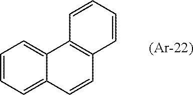 [SMILES: c1ccc2c(c1)ccc1ccccc12] 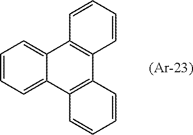 [SMILES: c1ccc2c(c1)c1ccccc1c1ccccc21] 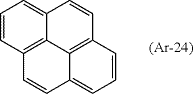 [SMILES: c1cc2ccc3cccc4ccc(c1)c2c34] 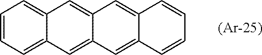 [SMILES: c1ccc2cc3cc4ccccc4cc3cc2c1] 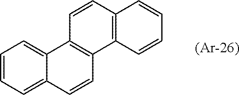 [SMILES: c1ccc2c(c1)ccc1c3ccccc3ccc21] 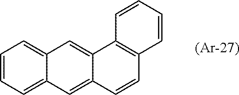 [SMILES: c1ccc2cc3c(ccc4ccccc43)cc2c1]
.

9. A light-emitting device comprising the organic compound according to claim 7.

10. A light-emitting apparatus comprising:
the light-emitting device according to claim 9; and
at least one of a transistor and a substrate.

11. An electronic device comprising:
the light-emitting apparatus according to claim 10; and
at least one of a sensor unit, an input unit, and a communication unit.

12. An organic compound represented by Structural Formula (100), Structural Formula (101), or Structural Formula (113): 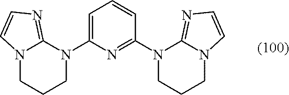 [SMILES: c1cc(N2CCCn3ccnc32)nc(N2CCCn3ccnc32)c1] 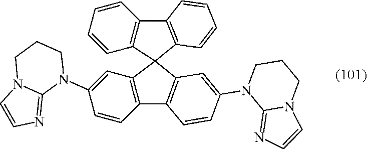 [SMILES: c1ccc2c(c1)-c1ccccc1C21c2cc(N3CCCn4ccnc43)ccc2-c2ccc(N3CCCn4ccnc43)cc21] 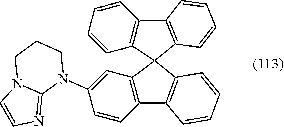 [SMILES: c1ccc2c(c1)-c1ccccc1C21c2ccccc2-c2ccc(N3CCCn4ccnc43)cc21]
.

13. A light-emitting device comprising the organic compound according to claim 12.

14. A light-emitting apparatus comprising:
the light-emitting device according to claim 13; and
at least one of a transistor and a substrate.

15. An electronic device comprising:
the light-emitting apparatus according to claim 14; and
at least one of a sensor unit, an input unit, and a communication unit.
